(19) United States
(12) Patent Application Publication
(10) Pub. No.: US 2023/0248377 A1
(43) Pub. Date: Aug. 10, 2023

(54) SUCTION CATHETER SYSTEMS WITH DESIGNS ALLOWING IMPROVED ASPIRATION AND EVALUATION OF ASPIRATION CONDITION

(71) Applicant: MIVI Neuroscience, Inc., Eden Prairie, MN (US)

(72) Inventors: John Wainwright, Foothill Ranch, CA (US); Mayra Contreras, Santa Ana, CA (US); Matthew F. Ogle, Edina, MN (US)

(21) Appl. No.: 17/667,828

(22) Filed: Feb. 9, 2022

(51) Int. Cl. A61B 17/22

(52) U.S. Cl. CPC A61B 17/22; A61B 2017/22079; A61B 2017/00292

(57) ABSTRACT

An aspiration thrombectomy system is described with an aspiration catheter assembly having fittings interfaced with conduit and a pump. The aspiration catheter assembly can include a guide catheter and an aspiration catheter. The aspiration catheter can be introduced with a proximal end of a tubular element placed within the guide catheter to form a suction lumen extending through the aspiration catheter and a portion of the guide catheter. A connection section forming an effective seal between the aspiration catheter and the inner wall of the guide catheter can be fixed or extendable through actuation or release of a self-extending structure. The aspiration catheter can be positioned into an artery with a distal opening positioned proximal to a clot. The fittings can include a filter for removing thrombus from the aspiration flow. The fittings and/or the tubing adjacent the pump can include a flow meter for measuring flow to the pump. The fittings can include a pressure sensor for measuring pressure in the fittings. In addition, the aspiration catheter can have one or more pressure sensors integrated into the wall of the catheter with each configured to measure pressure in the interior or exterior of the catheter. The aspiration catheter and/or flow through the catheter can be manipulated based on pressure and flow measurements.

### FIELD OF THE INVENTION

The invention relates to aspiration catheter systems designed with fittings designed for efficient and safe operation of the aspiration treatment for use in bodily vessels with tortuous paths, such as cerebral arteries. In particular, the invention relates to suction catheter systems comprising a guide catheter and a suction extension slidably disposed within the guide catheter and to fittings allowing for efficient evaluation of the processing and reuse of the suction extension.

### BACKGROUND OF THE INVENTION

Procedures in blood vessels of the brain are gaining use as an approach for ameliorating acute stroke events or other interventions in blood vessels in the brain. Blood vessels in the brain follow particularly tortuous paths which can increase the difficulty of reaching target locations in these vessels. Other vessels in a patient can also follow winding paths that increase the difficulty of reaching target locations.

Aspiration catheters have found use with respect to removal of clots from vessels. Furthermore, a significant reason for ischemic injury during percutaneous procedures can be generation of emboli that block smaller distal vessels. Aspiration catheters used alone or with embolic protection device can be effective to capture emboli generated during procedures. The delivery of effective devices to the small blood vessels of the brain to remove clots and/or to capture emboli remains challenging.

Ischemic strokes can be caused by clots within a cerebral artery. The clots block blood flow, and the blocked blood flow can deprive brain tissue of its blood supply. The clots can be thrombus that forms locally or an embolus that migrated from another location to the place of vessel obstruction. To reduce the effects of the cut off in blood supply to the tissue, time is an important factor. In particular, it is desirable to restore blood flow in as short of a period of time as possible. The cerebral artery system is a highly branched system of blood vessels connected to the interior carotid arteries. The cerebral arteries are also very circuitous. Medical treatment devices should be able to navigate along the circuitous route posed by the cerebral arteries for placement into the cerebral arteries.

### SUMMARY OF THE INVENTION

In a first aspect, the invention relates to an aspiration catheter system comprising an aspiration catheter and a proximal fitting. The aspiration catheter generally comprises a distal aspiration opening and lumen ending at the distal aspiration opening. The wall of the aspiration catheter can comprise a pressure sensor with an output value related to pressure at the pressure sensor. The proximal fitting can be connected with an aspiration source to provide negative pressure at the distal aspiration opening.

In a further aspect, the invention relates to a method for performing aspiration from a bodily vessel. The method can comprise applying aspiration with an aspiration catheter within a vessel. The method can comprise monitoring pressure exterior to the aspiration catheter within the vessel to obtain a pressure value. The method can comprise adjusting aspiration negative pressures or stopping aspiration based on the measured pressure.

In a further aspect, the invention relates to an aspiration catheter system comprising an aspiration catheter assembly, fittings, a pump, a conduit, and a flow meter. The aspiration catheter assembly generally comprises a lumen extending from a proximal end with a connector, to a distal opening. The fittings can comprise a branched manifold with a first branch comprising a hemostatic valve and a second branch comprising a connector. The aspiration catheter system generally comprises a conduit connected to the pump and to the connector of the second branch. The flow meter can be connected the connector or to the fittings, and a controller. The flow meter can be configured to measure flow from or to the aspiration catheter. The controller can comprise one or more displays configured to display values related to the flow.

In another aspect, the invention relates to an aspiration catheter system comprising a guide catheter, an aspiration catheter, and proximal fittings. The guide catheter can comprise a proximal hub and a tubular section connected to the proximal hub with a lumen extending from the proximal hub to a distal opening. The aspiration catheter can comprise a connecting section, a distal aspiration section with an aspiration lumen and a distal aspiration opening, and a proximal control structure. When positioned in the guide catheter as a suction extension, the distal aspiration section can extend from the distal opening of the guide catheter with the connecting section engaging with the inner wall of the guide catheter within the lumen and with the proximal control structure extending through the hub of the guide catheter. The connecting section can comprise a tubular element with a lumen open at a proximal end and fluidly connecting with the aspiration lumen of the distal aspiration section, a framework around the tubular element having a low profile configuration and a higher profile engagement configuration. A transition from the low profile configuration to the higher profile engagement configuration can be actuated through engagement with the framework using the proximal control structure having a translatable element between a proximal end and the framework, or through release of constraints on the framework, the framework being self-actuating. The proximal fittings can be connected to the proximal hub of the guide catheter and can comprise a manifold with a hemostatic valve configured to provide for passage of the proximal control structure to pass through the hemostatic valve when the distal aspiration section is extending in a distal orientation from the distal opening of the guide catheter, and with a branch connecting to an aspiration source with a configuration to apply negative pressure along an aspiration lumen extending from the negative pressure source through the manifold, a portion of the guide catheter to the lumen of the aspiration catheter to apply suction at the distal aspiration opening.

In a further aspect, the invention relates to a method for applying aspiration within a bodily vessel with an aspiration catheter system. For performance of the method, the aspiration catheter system can comprise a guide catheter, an aspiration catheter comprising an engagement section, a proximal control structure, and a distal aspiration tube, and proximal fittings. The proximal fittings can comprise a hemostatic valve and a connection to an aspiration source. The engagement section has a low profile configuration and an extended configuration. The method can comprise positioning a distal aspiration opening of the aspiration catheter at a desired position within a vessel within a patient. The method can comprise, after positioning the aspiration catheter, transitioning the engagement section to an extended configuration forming a seal with the wall of the guide catheter. The transitioning can comprise delivering electrical current through the proximal control structure or moving a corewire within the proximal control structure. The method can comprise applying suction at the distal aspiration opening to perform a desired procedure in the vessel.

In another aspect, the invention pertains to an aspiration thrombectomy system comprising an aspiration catheter assembly, fittings, a pump and a conduit to the pump and to a connector of the fitting. The aspiration catheter assembly generally comprises a suction lumen extending from a proximal end to a distal opening, in which the proximal end comprises a connector. The fittings can comprise a branched manifold with a first branch comprising a hemostatic valve and a second branch comprising the connector directly or indirectly connected with the conduit, in which the branched manifold is attached to the connector of the aspiration catheter assembly. The conduit can comprise tubing and a filter having an inlet and an outlet connected to the tubing, in which the filter comprises a filtration structure comprising electrodes for impedance/conductivity measurements. The electrodes of the filtration structure can be connected to a controller that provides current to the electrodes and measures fluctuations to the current to evaluate the electrical properties of material contacting the filter.

### BRIEF DESCRIPTION OF THE DRAWINGS

FIG. 1 is a side view of a suction catheter system comprising a guide catheter with a suction extension with the guide catheter shown as transparent to allow visualization of structure within the guide catheter.

FIG. 2 is a side view of an embodiment of a guide catheter extending from a luer fitting to a distal tip.

FIG. 3 is a fragmentary sectional view of a portion of the guide catheter of FIG. 2 between points 3-3 in FIG. 2 with the cross section taken along a plane through the central axis of the catheter.

FIG. 4 is a fragmentary sectional view of a portion of the guide catheter of FIG. 2 between points 4-4 in FIG. 2 with the cross section taken along a plane through the central axis of the catheter.

FIG. 5 is a side view of a branched hemostatic valve fitting suitable for connection with the luer fitting of the guide catheter of FIG. 2.

FIG. 6 is a side view of an embodiment of a section extension.

FIG. 7 is a top view of the suction extension of FIG. 6 with some hidden structure shown with dashed lines.

FIG. 8 is a sectional side view of the suction extension of FIG. 6 taken along line 8-8 of FIG. 7.

FIG. 9 is a fragmentary sectional view taken along line 9-9 of FIG. 6.

FIG. 10 is a fragmentary sectional view taken along line 10-10 of FIG. 6.

FIG. 11 is a fragmentary sectional view of the catheter of FIG. 11 taken along an orthogonal view indicated by line 11-11 of FIG. 9.

FIG. 12 is a sectional end view of the catheter of FIG. 6 taken along line 12-12 of FIG. 8.

FIG. 13 is a fragmentary side view of an alternative embodiment of a suction extension with the expanded insert showing the attachment of a control wire to the proximal portion using a coiled end portion of the control wire.

FIG. 14 is a sectional view taken along line 14-14 of FIG. 13.

FIG. 15 is a top view of an alternative embodiment of a suction extension in which a tubular extension has two tubular sections with different diameters connected by a taper section.

FIG. 16 is a sectional view of the alternative embodiment of the suction extension shown in FIG. 15, in which the section is taken along line 16-16 of FIG. 15.

FIG. 17A is a fragmentary side view of an alternative embodiment of the proximal end of the control structure in which a handle is attached to the control structure and the end of the control structure is twisted to restrict the movement of the handle relative to its position on the control structure.

FIG. 17B is a fragmentary side view of a section of catheter wall with a sensor positioned exterior to the catheter wall.

FIG. 17C is a fragmentary sectional view of a section of catheter wall with a sensor positioned interior to the catheter wall.

FIG. 18A is a fragmentary side view of a suction tip with a bend.

FIG. 18B is a fragmentary side view of a suction tip with a bend and an angled opening.

FIG. 18C is a fragmentary side view of a suction tip with a gentle curve.

FIG. 19A is a sectional side view of a connection section of an aspiration catheter with a thermally actuated seal surrounding a tubular feature of the connection section in a low profile configuration.

FIG. 19B is aside view of the thermal actuator for activating the thermal seal of FIG. 19A shown separated from the other elements of the connecting section.

FIG. 19C is a sectional side view the connection section of FIG. 19A in an extended configuration.

FIG. 19D is a side view of a corewire with an overtube.

FIG. 19E is a side perspective view of a manual control mechanism with a plurality of selectable stops for actuating the corewire of FIG. 19D.

FIG. 19F is a fragmentary side view of an alternative embodiment of a manual control mechanism for actuating the corewire of FIG. 19D, in which the corewire terminates with a knob that can be manually controlled by an operator.

FIG. 19G is a sectional side view of a connection section comprising a manually activated seal surrounding a tubular feature of the connecting section shown in an unextended configuration, in which a frame comprises bendable arm, a fixed proximal edge and a distal edge connected to a sliding corewire.

FIG. 19H is a side view of the seal frame of FIG. 19G shown separated from the other portions of the connecting section.

FIG. 19I is a sectional side view of the connecting section of FIG. 19G in an extended configuration.

FIG. 19J is a sectional side view of a connection section with an alternative embodiment of a manually activated seal surrounding a tubular feature of the connection section shown in an unextended configuration, in which the actuation element comprises a stent-like frame fixed at a distal edge and connected at its proximal edge.

FIG. 19K is a sectional side view of the connection section of FIG. 19J shown in an extended configuration.

FIG. 20A is a sectional side view of a connection section with a self-actuating seal surrounding a tubular feature of the connection section with an sheath surrounding the self-actuating seal.

FIG. 20B is a sectional side view of the connection section of FIG. 20A with the sheath withdrawn.

FIG. 20C is a sectional view of the connection section shown in FIG. 20A, in which the section is taken along the line A-A of FIG. 20A.

FIG. 20D is a sectional view of the connection section shown in FIG. 20A, in which the section is taken along the line B-B of FIG. 20A.

FIG. 21 is a sectional end view of a connecting section of a suction extension interfacing with engagement section of a guide catheter with a non-circular cross section.

FIG. 22 is a schematic depiction of a collection of medical devices that can be used together or in selected sub-combinations for selected percutaneous procedures in bodily vessels including a suction system as described herein.

FIG. 23 is a fragmentary side view of proximal fittings shown with two separated components adjacent a guide catheter in which the two components are a Y-branch manifold and an extended hemostatic fitting.

FIG. 24 is a fragmentary side view of a first alternative embodiment with a single non-branched component with a proximal hemostatic valve adjacent a guide catheter, which is suitable for use with a docking branched manifold.

FIG. 25 is a fragmentary side view of another alternative embodiment of proximal fittings attached to a guide catheter with a three branch manifold extending from the guide catheter and an extended hemostatic fitting attached to one branch.

FIG. 26 is a fragmentary side view of a further alternative embodiment of proximal fittings extending from a guide catheter in which the fittings comprise a Y-branch manifold, a T-branch manifold connected to one branch of the Y and an extended hemostatic fitting extending from the straight branch of the T-branch and a negative pressure device attached along the T-branched conduit.

FIG. 27 is a perspective view of a Y-branch manifold adapted for connection with a pump and attached to a pressure sensor and to a solenoid valve.

FIG. 28 is a side view of a Y-branch manifold attached to a tubular fitting adapted with a pressure sensor having an electronic connector and a flow sensor having an electronic connector.

FIG. 29 is a side view of a Y-branch manifold with a terminal pressure sensor along one branch and with an electrical connector for connection with the pressure sensor.

FIG. 30 is a side view of a first embodiment of a docking branched manifold with a branching fluid delivery channel.

FIG. 31A is a side view of an alternative embodiment of a docking branched manifold with a docking element FIG. 31B is a side view of the docking branched manifold of FIG. 31A with a negative pressure device attached to one of the branches of the branched manifold.

FIG. 31C is a fragmentary sectional view of the docking branched manifold of FIG. 31A showing the distal end with the docking element.

FIG. 32 is a side view of a guide catheter, a first fitting elements with a branched manifold that form part of the proximal fitting for the aspiration system, and a docking branched manifold, in which a hidden docking element is shown in dashed lines.

FIG. 33 is a side view of a guide catheter, an alternative embodiment of a first fitting element with a branched manifold with an additional branch, and a docking branched manifold.

FIG. 34A is a side view of the fitting components connected to the guide catheter as shown in FIG. 31A with a loaded suction extension in which the control structure of the suction extension is shown exiting the proximal end of the fittings.

FIG. 34B is a fragmentary sectional view of a portion of the first fitting element and the docking branched manifold of FIG. 34A where the section is taken through a central axis of the lumen, in which the suction extension is in a docked position engaging the docking branched manifold.

FIG. 34C is a fragmentary sectional view of a portion of the first fitting element and the docking branched manifolds of FIG. 34A as shown in FIG. 34B except that the suction extension is in an un-docketed position.

FIG. 35A is a side view of an assembled pin vise handle.

FIG. 35B is a cross-sectional view of a collet separated from the pin vise of FIG. 35A.

FIG. 35C is a side view of a pin vise with a head removed.

FIG. 35D is an exploded view of the pin vise handle of FIG. 35A with the components separated along the central axis.

FIG. 36A is an exploded perspective view of a filter with a corrugated filer element.

FIG. 36B is a side view of the filter of FIG. 36A.

FIG. 36C is a perspective view illustrating flow through the filter element of FIG. 36A.

FIG. 37A is an exploded perspective view of a filter with fiber based filter element.

FIG. 37B is an exploded perspective view of a filter with a wad of sheet like filter material.

FIG. 38A is an exploded perspective view of a filter with a screen filter element.

FIG. 38B is a side view of the filter of FIG. 38A.

FIG. 39A is side view of a filter with a screen filter element in a compartment secured below a cap.

FIG. 39B is an exploded side view of the filter of FIG. 39A.

FIG. 39C is a cross-sectional view of the cap of the filter of FIG. 39A.

FIG. 39D is a cross-sectional view of the filter of FIG. 39A.

FIG. 39E is a side view of a filter with a screen filter element in a compartment secured below a cap with an electrical connection to a controller.

FIG. 39F is an exploded side view of the filter of FIG. 39E with electrodes encircling filter element and an electrical connecting exiting through a sealed hole in the filter body.

FIG. 39G is a cross-sectional view of the filter of 39E with electrodes encircling filter element and an electrical connecting exiting through a sealed hole in the filter body.

FIG. 40 is a side view of a flow meter.

FIG. 41 is a side view of a pressure sensor.

FIG. 42 is cross-sectional view of a flow meter with a paddle wheel.

FIG. 43 is a cross-sectional view of a pressure sensor.

FIG. 44A is a side view of an embodiment of a proximal fitting for an aspiration system having a guide catheter, a first fitting element with a branched manifold where a first branch has a pressure sensor, a flow sensor, a filter, and a negative pressure source.

FIG. 44B is an alternative embodiment of the aspiration system of FIG. 44A in which the pressure sensor is on a second branch of the branched manifold.

FIG. 45 is a fragmentary view of an embodiment of an aspiration system from a position in the neuro-vasculature to the proximal fittings.

FIG. 46 is a schematic depiction of a human patient with alternative access approaches for directing catheters into the blood vessels of the brain.

FIG. 47 is a view within a branched blood vessel section showing the delivery of medical devices along a guidewire from a guide catheter to a clot. Inserts show expanded views of two internal sections of the guide catheter.

FIG. 48 is a schematic view in a section of blood vessel of a suction system being used to remove a clot.

FIG. 49 is a schematic view in a section of blood vessel with a suction system positioned upstream from a clot and a fiber based filter deployed downstream from the clot.

FIG. 50 is a schematic view of the section of blood vessel of FIG. 49 with the fiber based filter being drawn toward the suction tip to draw the clot to the tip for facilitating removal of the clot.

FIG. 51 is a schematic view of a section of blood vessel with a suction system positioned upstream from a clot, a fiber based filter deployed downstream from the clot and another medical device positioned at the clot.

FIG. 52 is a schematic view of the section of blood vessel of FIG. 51 with the various medical devices being used in concert for the removal of the clot.

FIG. 53 is a fragmentary view of a treatment system extending from a position in the neuro-vasculature to the proximal fittings shown following application of suction and optionally other procedural steps to remove a clot, with an insert in the figure showing a sectional view of a tubular extension within a guide catheter.

FIG. 54 is a fragmentary view of the distal portion of the treatment system of FIG. 53 in which the tubular extension is withdrawn into the guide catheter, with an insert of the figure showing sectional view of the distal end of the tubular extension within the guide catheter.

FIG. 55 is a fragmentary view of the proximal end of the treatment system of FIG. 53 in which the tubular extension is withdrawn sufficiently such that a connection section of the suction extension is within proximal fittings external to the guide catheter as shown in the sectional view of the figure insert.

FIG. 56 is a fragmentary view of the proximal end of the treatment system of FIG. 53 in which the tubular extension is withdrawn from the guide catheter but remains enclosed in the proximal fittings with sealed hemostatic valves, with a left figure insert showing a sectional view of the distal end of tubular extension within a Y-branch manifold (and an alternative placement of the distal extension within extended hemostatic fitting noted with a dashed line) and a right figure insert showing the connection section of the suction extension within an extended hemostatic fitting with a control wire extending through a hemostatic valve.

FIG. 57 is a fragmentary view of the proximal end of the treatment system of FIG. 53 in which the tubular extension is withdrawn from the guide catheter but remains enclosed in the proximal fittings with sealed hemostatic valves, with a left figure insert showing a sectional view of the distal end of tubular extension within a Y-branch manifold (and an alternative placement of the distal extension within extended hemostatic fitting noted with a dashed line) and a right figure insert showing the connection section of the suction extension within an extended hemostatic fitting with a branched manifold connecting to the hemostatic valve and a control wire extending through a hemostatic valve of the branched manifold.

FIG. 58 is a fragmentary view of the proximal end of the treatment system of FIG. 53 in which the tubular extension having at least a portion of a clot at the distal end is docked in a branched manifold and is fully withdrawn from the sealed hemostatic valve of the treatment system.

FIG. 59 depicts an integrated display with the display showing patient imaging and windows indicating pressure and flow.

### DETAILED DESCRIPTION OF THE INVENTION

Additional improvements are provided for an aspiration catheter system that provides for more reliable control of the aspiration processes that are particularly suitable for acute stroke treatment. In particular, a filter can be positioned in the proximal fittings of the catheter system to remove thrombus from the aspiration flow at a further distance from a pump to allow for the maintenance of strong aspiration pressures during clot removal. Furthermore, the inclusion of a flow meter within the proximal fitting or near the pump provides valuable information on the status of the aspiration process to allow for better control of the process through an understanding of the status of the clot removal. The improved designs of the proximal fitting can be combined with other significant aspiration catheter system designs for further overall improvement of the aspiration process from a clinical perspective. In some embodiments, the catheter is designed with a pressure sensor in the wall of the aspiration catheter to provide for pressure measurements in the blood vessel and/or within the catheter. In further embodiments, a flow meter can be placed in the catheter system, such as near the guide catheter proximal end. Alternative designs of an aspiration catheter are provided that allow for an extendable connection section to engage an aspiration catheter functioning as a suction extension with the wall of a guide catheter. An electronically controlled valve connected to the high pressure tubing to the pump can provide good response times and improved control of aspiration with less variation in pressure through turning aspiration on and off without powering the pump on and off. A continuously variable valve can adjust the strength of the aspiration, optionally with integrated control based on sensor measurements. An extendable connection section can be actuatable or self-extending upon release. An extendable connection section can provide for easy positioning while resulting in a good seal against the guide catheter wall.

The designs of the proximal fittings described herein generally can be effectively used in various aspiration catheter systems. But the fitting improvements can be particularly advantageous for systems with a aspiration catheter or suction extension that is designed to insert with its proximal end into a guide catheter with its distal tip extending past the guide catheter. Such an aspiration catheter system forms a single suction lumen extending through the aspiration catheter/suction extension through the guide catheter from the distal end of the aspiration catheter/suction extension to the distal end of the guide catheter. Depending on the perspective, the component of the aspiration catheter system is reasonably referred to as a suction extension for its role extending the suction lumen past the distal end of the guide catheter or as an aspiration catheter since it performs the role of an aspiration catheter although it is fully inserted in the patient during aspiration. Thus, the terminology is used interchangeably. Alternative embodiments for the aspiration catheter configured as a suction extension are also described herein.

Previous improvements in the proximal fittings for aspiration thrombectomy systems allow for effective use of an aspiration catheter with a tether and a sealing section to provide that the catheter is delivered into a guide catheter with a distal section extending out from the distal tip of the guide catheter and with the sealing section within the guide catheter for form a suction lumen comprising a lumen through the aspiration catheter and a section of the guide catheter. The previous improvements provided for allowing the placement of the entire length of the aspiration catheter within the proximal fittings separated from the guide catheter but behind a hemostatic valve as well as removal of the aspiration catheter from behind the hemostatic valve while maintaining a continuous fluid connection to the aspiration catheter such that the aspiration catheter can be cleared of obstructions and efficiently returned behind the hemostatic valve for performing additional aspiration positioned within the guide catheter. The additional improvements described herein provide for more efficient aspiration and evaluation of the status of the aspiration system.

While pressure sensors can provide useful information regarding the status of an aspiration process, this information can be incomplete and therefor potentially ambiguous. As described herein, a flow meter is provided in the proximal fittings to provide additional information that can clarify process status. For example, while a pressure change may suggest some blockage, a review of the flow status can provide valuable information on the degree of blockage and potential changes over time that may be more sensitive than pressure fluctuations alone. Also, a sudden increase in flow can indicate movement or clearance of a clot, which can prompt the medical professional to check for the clot in the filter. Various flow meter designs can be adapted for this purpose, such as commercial ultrasonic flow meters, which can be conveniently clipped onto flow structures, such as piping, tubing and the like, or coriolis flow meters that are connected to the tubing with flow passing through the device. Reusable flow meters can be placed adjacent the pump away from sterile components. In some embodiments, a flow meter can be made integral with the guide catheter, such as placement in or adjacent the catheter hub or part of the proximal fitting within sterile, disposable components.

While various negative pressure devices, such as syringes or the like, can be used to aspirate blood from an aspiration catheter, the use of a medical pump is desirable for providing a steady and reproducible negative pressure, which can be especially significant for acute stroke intervention. Medical grade pumps are commercially available for use for various pulmonary, surgical and vascular procedures. To protect the pump, these pumps generally have a large canister at the pump housing to collect liquids and a filter to catch bacteria and any residue that escapes being caught in the canister. Nevertheless, between the pump and the aspiration catheter system components there is generally a relatively long section of high pressure, medical grade tubing. The aspiration system is sterile due to access to a patient's vasculature, and the pump is not particularly sterile, due to various practical constraints. To allow for comfort of connecting these sterile and non-sterile components the long tubing, generally at least six feet long, is provided to create a practical division of these different environments.

As described herein, pressure transducers can be incorporated into the wall of the aspiration catheter to directly measure pressure within the patient. Depending on the orientation of the pressure transducer, the pressure can be measured within the catheter or exterior to the catheter, and multiple transducers can be used to measure pressures both in the catheter and outside of the catheter. The pressure transducers can transmit wirelessly a value related to the pressure for use of the pressure, or a wire with the signal can be associated with the other catheter structures to lead the signal wire to a location exterior to the patient. In general, these pressure measurements can be associated with any aspiration catheter design, and more detailed descriptions are provided for embodiments in which the aspiration lumen involves a portion of a guide catheter lumen. A measurement of the pressure outside of the aspiration catheter can provide information on whether a particularly pressure in the vessel could be applying more force than desired on the vessel wall, which may prompt a change in flow or stoppage of the aspiration.

The use of a pressure sensor in an aspiration catheter system is described in published U.S. patent applications 2017/0181760 to Look et al., entitled “Aspiration Monitoring System and Method,” and 2019/0183517 to Ogle, entitled “Suction Catheter Systems for Applying Effective Aspiration in Remote Vessels, Especially Cerebral Arteries” both of which are incorporated herein by reference. Both of these references discuss pressure sensors associated the proximal fittings. These references do not discuss measurement of pressure in the vessel.

The high-pressure tubing is relatively small diameter, which is not an issue except when a clot is moving through the tubing. A clot, even if effectively drawn though the high-pressure tubing, significantly reduces the negative pressure in the aspiration system while it travels to the large canister at the pump that collects fluid. With the long length of the tubing, a non-trivial amount of time is need for the clot to get to the large canister to trap the clot. In improved systems described herein, a relatively small but effective filter is provided to catch the clot at or near the proximal end of the high-pressure tubing. The filter generally is connected to the fittings on one side and to the high-pressure tubing on the other side, but in some embodiments the filter can be attached within 12 centimeters of the fittings connected with a corresponding section of tubing or other flow conduit. In some sense, the end of the fittings can be identified to be the connection to the filter, and the high-pressure tubing is identified as a section that divides the sterile environment immediately around the patient from the clean, but not necessarily sterile environment around the pump. The filter generally has an effective increased diameter over a limited length and an internal structure or material to catch clots in the blood without significantly limiting flow. The filter can be useful also from the perspective of providing information on the status of the clot, i.e., whether it has been identified as being captured and in a safe location, whether or not the filter is used to avoid clogging of the high pressure tubing. The filter can be provided sterile, and use of the filter can keep significant clots out of the high-pressure tubing.

The filter housing can be made transparent to allow for visual inspection of the status of clot capture. Additionally or alternatively, the filter can be instrumented to allow for automated detection of the presence of a clot. Measurements have been made on the electrical conduction properties of clots containing red blood cells. See, Liu et al., “Bioimpedance as a Means to Quantify Clot Composition and Guide Selection of Thrombectomy Devices,” International Stroke Conference 2021 Poster Abstracts, March 2021, Stroke. 2021; 52: AP782 (//doi.org/10.1161/str.52.suppl_1.P782), incorporated herein by reference. While the measurements in Liu article were made under more controlled conditions with a specific configuration, less controlled measurement conditions should generally provide basic information such as the presence or absence of a clot and likely some parameters related to amount or nature of the clot. Clinical experience with this data may allow for further correlations of data. Automated detection of clot presence alone can be valuable since the health care professional can be alerted without needing to look in the filter, which can significantly facilitate the procedure. The filter element can be instrumented to facilitate electrical measurements to provide information on clot status.

Such a structure can provide significantly improved control over the aspiration procedure and/or provide visualization of clot capture. The use of a filter in the sterile vicinity of the fittings can provide improved procedures with any aspiration catheter design, such as the aspiration catheters described in U.S. Pat. No. 9,662,129 to Galdonik et al., entitled “Aspiration Catheters for Thrombus Removal,” incorporated herein by reference, as well as the designs described herein. Filters, traps and the like can be used to protect pumps and other negative pressure devices. Such devices can be placed in vicinity to the pump or the like. For example, such a use is described in U.S. Pat. No. 10,779,855 to Garrison, entitled “Methods and Systems for Treatment of Acute Ischemic Stroke,: incorporated herein by reference.

Aspiration thrombectomy procedures have been performed clinically with relatively steady application of aspiration turned on and off at desired times. Some model studies have suggested that improved suction can be obtained with cyclic aspiration, such as with aspiration pulses at 0.5 to 5 hertz. See, Good et al., “Hydrodynamics in Acute Ischemic Stroke Catheters Under Static and Cyclic Aspiration Conditions,” Cardiovascular Engineering and Technology, Vol. 11 (6), December 2020, 689-698, incorporated herein by reference. In a clinical setting, while force is significant, the effects on the force on clots should be taken into consideration. Cyclic aspiration can be applied using the catheter systems described herein. Pump technology to implement the cyclic aspiration is described in U.S. Pat. No. 10,390,926 to Janardhan et al., entitled “Aspiration Devices and Methods,” incorporated herein by reference. To avoid fragmentation and embolization of clots under cyclic aspiration, it may be desirable to employ a distal filter device, as described further below, when cyclic aspiration is applied. Furthermore, flow to the pump can be controlled additionally using a valve, such as an electronically controlled valve. In some embodiments, the valve can be a solenoid valve mounted external to the tubing with closing of the valve resulting in a pinching closed of the tubing. Other valves, such as diaphragm valves or mechanical leaflet valves can be used alternatively, which may or may not be pinch valves in isolation from the flow. Commercial valves can be adapted for connecting to the aspiration tubing. An appropriate controller can operate the valve automatically in response to sensor readings and/or manually.

A suction catheter system can include a guide catheter adapted with an aspiration catheter configured to function as a suction extension of the guide catheter having a narrower distal tube that can provide suction with a high flow rate. This two-piece system provides an advantage of strong suction ability, while also providing some flexibility with respect to efficient performance of the procedure while leaving the guide catheter in position. Fitting designs are described that provide for removal of the suction extension for quick clearing of debris from the suction extension to allow reinsertion of the suction extension while maintaining the guide catheter in position. Specifically, a fitting element can engage the proximal opening of the suction extension at a docking structure to provide for clearing of the suction extension. In additional or alternative embodiments, proximal fittings can be provided to allow withdrawal of the tubular portion (tubular extension) of the suction extension from the guide catheter without bringing the tubular extension of the suction extension through a hemostatic valve. Methods are described in which a docking fitting docked at the end of the suction extension provide for contact with the fitting while providing for blowing debris from the suction extension, such that the cleared suction extension can then be reinserted through a hemostatic valve and reinserted for the application of additional suction. In a significant number of procedures, the suction nozzle can be cleared one or more times to reopen the clogged vessel. Efficient cleaning of the suction extension can significantly facilitate the procedure.

In some embodiments, the suction extension has a connecting section that has a noncircular circumference interfacing with the inner surface of the guide catheter with contact at two locations to provide an effective fluid seal while providing for translation of the suction extension within the guide catheter. In some embodiments, the connection section can have a radially extendable component to form the seal, which can have a circular or noncircular circumference. In alternative or additional embodiments, the guide catheter can have a distal portion of a tubular element that has a narrower diameter or a tab(s) that effectively limits the movement of the suction extension in a distal direction. Methods are described for the use of the suction catheter system such that the tubular extension of the suction extension that provides part of the aspiration lumen remains in a sealed configuration with respect to the guide catheter lumen, in some embodiments, for the entire period in which the guide catheter is within a patient Improved processing can be guided through the use of real time line pressure measurements with a pressure transducer associated with appropriate back end tools. Suction catheters can be used advantageously for the removal of thrombus and emboli from bodily vessels, such as arteries. Some vessels can have a narrow diameter, and treatment locations can be downstream along a circuitous path, and for such vessels there are constraints on the catheter structures able to reach the treatment locations in the vessel.

The designs described herein comprise a slidable suction extension that can be adapted for use in conjunction with a corresponding guide catheter, which forms a significant portion of the overall suction lumen when suction extension is deployed from the distal end of the guide catheter. In some embodiments herein, fittings positioned at the proximal end of the catheter system can be designed to improve the medical procedures to allow for more efficient performance of the revascularization of blocked vessels. Improved efficiencies can reduce time that the patient has catheters in their vasculature and decrease health care professional time devoted to the procedure. While the suction catheter system can be used in any suitable vessels of the body, the system can be particularly desirable in cerebral blood vessels, such as for the treatment of acute stroke. The suction catheter system can be effectively used as a standalone suction catheter for thrombus removal. Furthermore, the suction catheter system can be effective as a component of a thrombectomy treatment system or other medical system to provide suction with the use of other medical devices, such as a clot engagement device, to disrupt thrombus and/or a filter structure that can catch emboli generated in the procedure as well as to be used to pull toward the suction catheter system. The treatment system can be effectively designed for stroke treatment.

Less invasive procedures, which are commonly referred to in the art as minimally invasive procedures, are desirable in the medical context when appropriate to reduce patient recovery times and in many cases to improve outcomes. In particular, less invasive procedures are commonly performed in the vasculature using catheter-based system for reaching remote locations in a selected blood vessel for the performance of various treatment processes. These procedures can also be referred to as percutaneous procedures or transluminal procedures, in contrast with open surgical procedures, to emphasize the delivery through a vessel lumen. The discussion herein focuses on treatment of ischemic stroke since the devices can be particularly effective to treat these clinically important conditions, although the devices can be used in other procedures both in the vasculature and in other bodily vessels. Patients include humans and can include other mammals, such as pet animals and farm animals. The terms proximal and distal are used in their conventional sense in the art, i.e., proximal refers to closer to the point of entry into the patient along the path in the vasculature or other vessel and distal refers to farther from the point of entry along the path in the vasculature.

A slidable suction extension generally comprises a connecting section that engages the inner wall of the guide catheter to make a suitably tight fit. The connecting section generally links a control structure, such as a control wire, extending in a proximal direction from the connecting section, and a tubular extension that extends from the control structure in a distal direction. The control structure generally extends outside of the patient to provide for positioning the suction extension with its distal tip near a treatment location in a blood vessel. The tubular extension, which may have an optional curved tip, can be tracked well over a guidewire to reach difficult to reach locations in a vessel.

The connection section of the suction extension can have fixed dimensions or an extendable outer diameter. Connection sections with non-circular cross sections and a relative large length along the axis of the catheter have achieved good performance and have been adopted for commercial embodiments of the Q™ Aspiration Catheter (MIVI Neuroscicence). The use of an extendable outer diameter can provide for use in different sized guide catheter while providing suitable engagement with the guide catheter and/or can provide for a tighter seal with the guide catheter with ease of relative positioning of the suction extension within the guide catheter. The extendable outer diameter can be actuatable, and an actuation cable can be used to control the actuation/deactivation process, such as through an electrical signal or a mechanical movement. A balloon actuateable embodiment and some other engageable locking mechanisms are described in published U.S. patent application 2017/0143938A1 to Ogle et al. (hereinafter the '938 application, now U.S. Pat. No. 10,716,915), entitled “Catheter Systems for Applying Effective Suction in Remote Vessels and Thrombectomy Procedures Facilitated by Catheter Systems,” incorporated herein by reference.

In some embodiments, the actuation cable can comprise electrical connections to transmit electrical signals to the connection section, such as to induce resistive heating to extend the connection section. In additional embodiments, the actuation cable can comprise a corewire and overtube such that relative movement of the corewire can move mechanical elements in the connection section to engage or disengage the connection section. The control structure for the connection section can be the same as the actuation cable, if present, or these can be separate elements. In some embodiments, structures developed for temporary stents can be adapted for this purpose. In further embodiments, self-actuating components can be used in which release of the self-actuating components can result in deployment of the extended connection section. For self-actuating embodiments, designs of self-actuating stent retrievers can be adapted for this purpose. For extendable embodiments, the mechanisms, for example a frame or the like, providing the extending function generally can be covered to protect the vessel wall and placed over a tubular polymer element that forms the inner lumen through the connecting section. The cover can be, for example with an elastomeric polymer sheet, a woven fabric, nanofiber spun structure, expanded PTFE or the like. The components of the extendable cuff of the connection section of the aspiration catheter can borrow features from stent retrievers and stent grafts with respect to entendable mesh structures and expandable covers.

For embodiments of aspiration catheters incorporating extendable connection sections, the aspiration catheter may be combined with guide catheters of different sizes which providing the ability to engage the guide catheter lumen to form a leak resistant seal creating an aspiration lumen extending through the aspiration catheter and a portion of the guide catheter lumen. Whether or not the guide catheter has a selectable size, the ability to transition the connection section between a low profile, disengaged configuration to an extended engaged configuration can provide for improved pushability during positioning of the aspiration catheter, such as relative to a fixed position guide catheter, while then being able to establish a good seal with the guide catheter. The engaged seal can have a relatively small point of engagement with the guide catheter wall in some embodiments, while in other embodiments there can be extended sections of engagement between the connection section and the guide catheter inner walls, such that less force may be applied to the guide catheter wall that may allow for sliding movement of the aspiration catheter within the guide catheter while the connection section is engaged. For appropriate embodiments, the connection section can remain in a low profile configuration until the aspiration catheter is placed at a desired position, and then with the aspiration catheter in position, the connection section can be transitions to engage the guide catheter in a fluid tight seal. In some embodiments, the connection section can be disengaged to move the aspiration catheter relative to the guide catheter either for repositioning of the aspiration catheter and/or for removal of the aspiration catheter for ending the procedure or for clot removal and replacement. In some sense, disengagement to allow self-actuation can be considered in effect a different form of actuation.

Since thrombus can be held at the distal tip of the suction extension during the application of suction to remove the clot form the vessel, it can be desirable to withdraw a tubular extension of the suction extension into the guide catheter with the application of suction to reduce the chance of embolization of thrombus and loss of emboli that can travel upstream in the vessels. To further reduce the risk of embolization, it can be desirable to fully remove the tubular extension from the guide catheter with the application of suction prior to removal of the guide catheter from the patient. In a significant fraction of procedures, it can be useful to clear the suction extension and reinsert the suction extension for the removal of additional thrombus for the vessel. To achieve the best outcomes, it can be effective to repeat the suction process, two, three or possibly more times.

Desirable proximal fittings at the back end of the catheter system are described that allow for the removal of the tubular extension from the guide catheter without passing the tubular extension of the suction extension through a hemostatic valve. Since the proximal end of the tubular extension generally is open, passage of the proximal end of the tubular extension through a hemostatic valve can expose the interior lumen of the tubular extension and potentially of the guide catheter to the ambient environment, which may or may not be desirable. Additional fitting elements can allow removal of the suction extension through a hemostatic valve for clearing of the catheter while maintaining the fitting on the suction extension at all times, so that the suction extension can be rapidly redeployed. A docking fitting can comprise a distal docking structure that allows for docking the proximal end of the suction extension into the docking structure in an effective fluid tight seal for removal together out from the hemostatic valve. As noted above, this clearing of the suction extension can be repeated more than once.

The proximal fittings provide for the hemostatic isolation of the interior of devices that are exposed to the interior of the blood vessels. A guide catheter then forms an integral component of the aspiration system that provides for introduction of additional components, including but not limited to the suction extension. The fitting can then provide for hemostatic introduction of such other components while also providing for connections to negative pressure devices, such as pumps or syringes, and possibly delivery ports for introduction of contrast dye, medications or other desirable fluids. IV contrast dye fluids are well known in the art. Medications can be delivered in a suitable liquid form. These fitting then provide for the relative movement of the suction nozzle within the guide catheter and out form the guide catheter as well as the other functions.

The control structure for positioning the suction extension can be a wire-like element as described further below. For desirably simple designs of the guide catheter and the suction extension, it can be possible to push the suction extension out the distal end of the guide catheter, which can make it difficult or impossible to retrieve the suction extension from the patient while leaving the guide catheter in place. Markings on the control structure can discourage this movement of the control structure, but a user may disregard the markings. To avoid this possibility, a handle or grip can be secured on the control structure. If appropriate based on the handle design, the control structure can be bent, twisted or otherwise distorted to render it difficult or impossible to remove the handle. The handle then can limit the distal extension of the suction extension within the guide catheter so that the suction extension cannot be extended out of the distal end of the guide catheter.

In some embodiments, suitable proximal fittings suitable for withdraw the tubular extension out from the guide catheter but within hemostatic isolation have a tubular portion of the fittings following a branch structure in which the tubular section has sufficient length to hold the suction extension within the isolated region behind a hemostatic valve but external to the tubular element of the guide catheter. Several suitable configurations are described below and other configurations can follow from the discussion of these embodiments. It can be noted that aspiration is generally applied from a separate branch of the fittings and that multiple branches can be provided in the overall manifold, which may or may not have separable components that are assembled for use. This isolation structure can provide for evaluation of the status of the nozzle prior to withdrawal from hemostatic isolation and can be used in combination with the fitting to provide for effective clearing of the suction extension outside of hemostatic isolation without disconnecting the suction extension for the appropriate fittings.

The measurement of the pressure in the proximal fittings can provide valuable information relative to the procedure. Potential structures for placement of the pressure sensor are discussed below. If the pressure is near zero in the proximal fittings, then the flow in the line to the pump is effectively unconstrained. It is observed that pressure with flow passing through the suction extension results in a measurable drop in pressure but still at a pressure significantly less than the pump pressure. If the suction extension is clogged with thrombus or if the suction extension is kinked, the measured pressure can approximate the pump pressure, which generally indicates that flow is essentially block within the catheter. Knowledge of the blockage can be used to significantly improve the procedure with respect to efficacy and safety. For example, if the blockage occurs early in the procedure, this may suggest kinking. Blockage later in the procedure can suggest blockage of the catheter with trapped thrombus, which generally instructs that contrast dye or other infusion liquids should not be delivered through the catheter since the pressure of delivery can thrust the thrombus, which had been blocking the catheter, deeper into the vasculature. A pressure transducer can be introduced in alternative ways. For example, a pressure transducer can be placed along the inner wall of a fitting of the manifold or on a tube connected to the fittings with a configuration to provide pressure measurements. The pressure sensor may or may not be sterile depending on the location.

For the treatment of strokes, treatment devices can be advanced through arteries to blood vessels of the brain. Blood vessels generally relevant for acute stroke treatment are downstream in the blood flow from the internal carotid arteries, and arteries generally branch and decrease in average diameter as the vessel proceeds in a downstream direction in the arterial vasculature. The body has a right internal carotid artery and a left internal carotid artery. For convenience, the blood vessels downstream from the internal carotid arteries are referred to herein as cerebral arteries. The cerebral arteries can be accessed with catheter based systems from, for example, a femoral artery in the groin, an artery in the arm, or the carotid artery in the neck using hemostatic procedures and appropriate fittings, such as those known in the art. The cerebral arteries are known to follow circuitous paths, and complications in tracking devices along the vessels also follows due to narrow diameters and branching of the vessels as well as potentially dangerous risks from damage to the blood vessel that can cause a hemorrhagic stroke condition. Nevertheless, it can be desirable to access tortuous narrow arteries for stroke treatment. The devices described herein are designed for advantageous use in these tortuous narrow cerebral vessels, but a person of ordinary skill in the art will recognize utility of these devices in other medical procedures.

The present suction catheter systems incorporate guide catheters adapted with a slidable suction extension suitable for cerebral procedures. In vascular procedures generally, a guide catheter can be used to facilitate the delivery of therapeutic devices while allowing for more rapid, accurate delivery with less risk to vessel walls through providing a protected channel leading most of the way to the treatment site. In the cerebral procedures, a guide catheter can be placed from exterior of the patient at the point of entry into the vasculature with the distal end of the guide catheter in a carotid artery or interior carotid artery. Thus, a guide catheter can provide a lumen to a location relatively near to a treatment site. In some embodiments, conventional guide catheters can be used to assemble the desired suction catheter systems, but in other embodiments, specific guide catheter designs are used to form the suction catheter system. The size of the guide catheter sets limits on the diameter of treatment structures delivered to the treatment site, but this is generally not a significant issue since extendable devices can be delivered in a lower profile configuration with subsequent deployments to an extended configuration and since the vessel sizes generally decrease in a distal direction from the guide catheter limiting the need for larger treatment devices. The suction devices described herein provide a suction extension that can protrude from the distal end of the guide catheter an adjustable amount through the positioning of a connecting section of the suction extension interfacing the suction extension with the interior walls of the lumen of the guide catheter. The connecting section can make a sufficiently tight seal with the guide catheter walls such that suction in the guide catheter lumen is transmitted along the lumen of the suction extension. Desirable degrees of suction can be obtained through the suction extension using suction applied at the proximal end of the guide catheter.

The suction extension generally comprises a connecting section, a control structure extending in a proximal direction from the connecting section, and a tubular extension extending in a distal direction from the connecting section. The suction extension generally interfaces with the guide catheter and can be designed to be positioned with its tip at a selected position distal to the guide catheter for the performance of a procedure at a selected location, such as near the location of thrombus occluding a cerebral vessel. Since the relative position of the treatment location and the distal end of the guide catheter generally vary for a specific medical situation, the degree in which the suction extension extends from the guide catheter can be adjusted through relative movement of the suction extension using the control structure, e.g. a control wire. The suction extension should move within the guide catheter lumen without the need for excessive force, which may be facilitated through the use of low friction polymers on one or both adjacent surfaces.

The connecting section of the suction extension provides for an interface with the inner wall of the guide catheter to prevent most or all flow around the connecting section that does not flow through the lumen of the suction extension while keeping at least a portion of the connecting section within the guide catheter and while providing for appropriately unproblematic sliding of the suction extension relative to the guide catheter within the patient's vasculature. Various embodiments of components forming such an interface are discussed in the '938 application cited above. A connecting section, referred to as a proximal portion in the '938 application, can have a non-cylindrical cross sectional shape. Such a non-cylindrical cross sectional shape can advantageously provide for contact with the guide catheter at two locations around the circumference along with a small clearance around the remaining section of the circumference of the connecting section. Contact with the inner lumen of the guide catheter applies some force on the connecting section that partially rounds out the circumference. This non-cylindrical shape for the connecting section allows for effective blockage of flow between the guide catheter wall and the connecting section while not inhibiting movement of the connecting section longitudinally to position the tip of the suction extension within the vasculature. The introduction of a non-cylindrical shape of a connection section is described in U.S. Pat. No. 10,478,535B2 to Ogle (hereinafter the '535 patent), entitled “Suction Catheter Systems for Applying Effective Aspiration in Remote Vessels, Especially Cerebral Arteries,” incorporated herein by reference.

The non-circular cross sectional shape of the connecting section of the suction extension can generally be described as oval. The oval can be characterized at least in part by a major axis along the longer dimension of the oval and a minor axis along the shorter dimension of the oval orthogonal to the longer dimension. The connecting section can then contact or approach very closely to the inner surface of the engagement section of the guide catheter at two locations associated with the points along the circumference associated with the major axis. Correspondingly, the non-circular cross section can be characterized by an average radius, and the average radius can provide an overall very small clearance with the guide catheter while still providing for desirable function.

To form the non-circular cross section, a bump can be formed through the connection of a control wire along a surface of the connecting section along with extra polymer that provides for the desired shape along with reinforcing the control wire connection with the connecting section. Additional embodiments of the connecting section structure with an oval cross section are described below. Thus, the non-circular shape of the connecting section cross section can be designed for its interface with the guide catheter consistently with the overall structure of the suction extension.

Also, since it is desirable to prevent the connecting section of the suction extension from exiting from the distal end of the guide catheter, the suction extension and/or catheter can be designed to limit the distal movement of the suction extension. Several different designs of guide catheter and/or suction extension features are described in the '938 application and the '535 patent. To simplify the guide catheter structure and/or to provide for use of a conventional guide catheter design, it can be desirable to use a guide catheter without any specific structural features that limit the distal movement of the suction extension. But then movement of the suction extension should be limited through motion of the control structure. Instructions to the user based on marking on the control structure are prone to user error that allow for over extension of the connection section of the suction extension out past the distal end of the guide catheter. The elements added to the control structure described herein prevent the user from over extending the suction extension.

In comparison with a suction catheter delivered through the guide catheter in which the suction flow is confined to the suction catheter, a significant length of the suction catheter is replaced with a control element in the suction catheter systems herein. This replacement of a significant length of a suction catheter with a control element results in a device that can have less friction when the tip of the suction catheter is advanced in the patient's vasculature since a control wire or other control element can offers less resistance for its movement. The tip of the suction extension can be given a curved tip to facilitate tracking of the device over a guidewire. With the designs described herein, a suction extension for aspiration with a curved tip for tracking the tip over a guidewire can be effectively guided to difficult to reach locations with the use of a control wire or other control element moving the slide portion at or near the distal end of the suction extension, and the design provides for good suction ability without sacrificing the ability to reach difficult to reach vessels, such as within cerebral vessels. While the suction extension is moved, the guide catheter portion of the suction lumen can remain in place

When suction is applied at or near the proximal end of the guide catheter with a suitable negative pressure device, fluid is sucked into a distal opening at the end of the suction extension. It has been found that strong suction can be transmitted through to the suction extension. A suction lumen extends from a negative pressure device, generally attached at a fitting associated with a proximal section, at or near the proximal end of the suction system through the guide catheter lumen to the suction extension and through the connecting section of the suction extension and the tubular extension of the suction extension to a distal opening. Suitable negative pressure devices include, for example, syringes, pumps or the like. The guide catheter can provide a large lumen as a significant section of the overall suction lumen. The effective suction lumen then can appear to have a large proximal section contributed by the guide catheter and a tapered distal section contributed by the suction extension, which can have one or more tapered segments.

The tubular extension of the suction extension has a lumen with a reduced diameter relative to the guide catheter lumen and good flexibility to provide for placement of its distal end into smaller vessels. The lumen of the tubular extension though is maintained at a sufficiently large diameter that provides for delivery of additional therapeutic devices through the lumen to the treatment location. The outer diameter at the tip of the suction extension generally is (diameter in mm=(Fr value)/3, Fr represents the French catheter scale) at least about 1.5Fr less than the outer diameter of the distal section of the guide catheter. The smaller diameter of the tubular extension can provide access to desirable vessels, such as cerebral vessels.

It was previously discovered that good suction properties could be obtained with a suction catheter with a stepped down diameter in a distal section. Thus, for example, the majority of the length of the suction catheter can be 6Fr outer diameter while a distal section may be 5Fr outer diameter, which roughly corresponding decreases in the inner diameters. Such a catheter can provide access into vessels suitable for a 5Fr catheter, but can provide significantly better suction than a suction catheter with a 5Fr catheter body along its entire length. Commercial stepped down suction catheters, such as Mi-Axus™ catheters (MIVI Neuroscience, Inc.) and ACE™ 64 catheters (Penumbra, Inc.) are finding good clinical results. The step down suction catheters and their use for thrombectomy procedures in cerebral arteries are described in U.S. Pat. No. 9,532,792 B2 to Galdonik et al. (hereinafter the '792 patent), entitled “Aspiration Catheters for Thrombus Removal,” incorporated herein by reference. While these catheters achieve better suction than catheters with constant diameters corresponding with the distal diameters, the present suction catheter systems with a sliding suction extension are found to provide better suction suggesting that the diameter over the majority of the suction lumen length contributes to a large extent to the suction provided at the distal opening of the suction lumen.

An initial part of a procedure using the devices described herein generally involves accessing the treatment location within the vasculature. Guidewires have been designed to facilitate access to difficult to reach locations. The term guidewire is used herein to refer broadly to wire structures that may or may not have internal structure are referred to as guidewires whether or not they are formed from a solid or woven metal, such as corewire-overtube integrated structures, coils or the like which may not have a closed inner lumen over at least a portion of the devices length.

In particular, with the devices described herein procedures can be performed to provide re-profusion in vessels that are blocked completely or partially with clots. Clots in cerebral arteries can cause strokes with corresponding serious consequences, and time generally is of the essence of treating these conditions. The suction extension with the guide catheter can be used to provide aspiration that can be useful to remove clots or fragments thereof. Thus, the suction extension combined with the guide catheter and negative pressure device can be used as stand-alone devices for thrombectomy procedures. However, the suction extension with aspiration can be effectively used as part of a treatment system comprising, for example, also a fiber based filter and/or other components to facilitate removal of a clot or portions thereof. The delivery catheter with the expandable tip is designed to facilitate access, so it is useful as a tool for the practice of various other procedures.

In some embodiments of the procedure, a guidewire can be placed at or near an occlusion and a guide catheter with a positionable suction extension can be placed in the vasculature upstream from the occlusion with the guidewire extending through the interior of the suction extension. If the suction catheter system is to be used alone, then the suction extension can be advanced using a control wire over the guidewire to a suitable position near the clot. Then, with or without removing the guidewire, suction can be initiated to suck the clot or a portion thereof into the distal opening or against the tip of the suction extension. Suction may or may not be continued as the suction extension and/or guide catheter are removed from the patient.

While suction with the suction extension can be effective as the only device for clot removal, additional treatment systems can combine other devices for use with the suction catheter system. In particular, a filter device can be used to provide both embolic protection as well as a tool to facilitate removal of the clot or portions thereof, which may involve direct engagement of the clot with the filter device. Fiber based filters/embolic protection systems have been developed that can be effectively used in the narrow vessels of interest. In particular, fiber-based filter systems with an appropriate actuation system can be used for delivery in a low profile configuration past an occlusion and deployed to provide protection from any clot fragments that may be released during the removal process.

During the removal process of the suction catheter system and potentially other components of the treatment system from the patient, aspiration generally is continued until risk for embolization of thrombus is sufficiently lowered. The suction extension may have thrombus within the lumen and/or trapped at the tip. The proximal end of a tubular section of the suction extension generally is open such that if the proximal end of the tubular extension is removed through a hemostatic valve, the suction lumen of the tubular extension can be exposed to the ambient environment. Since exposure of the lumen of the tubular extension still within the patient can be undesirable, fittings have been devised as described herein that allow parking of the tubular extension external to the guide catheter while still within isolated sections of the system external to the patient. Aspiration can be continued while the tubular extension is removed from the patient and parked in isolation from the ambient but external to the guide catheter.

In some procedures, it may be desirable to clear the tubular extension while it is removed from the patient. Once cleared, the tubular extension can be reintroduced into the patient to retrieve additional thrombi. In such procedures, a docking branched manifold can be configured to facilitate the rapid removal and cleaning of the tubular extension. It is desirable to return the extension catheter to the vascular before embolization of thrombus at the clot. The docking branched manifold generally has an input tubular segment and at least one Y-branch having a fitting connected at the end of one branch to a flow valve. The flow valve generally has at least a second port connected to a flush fluid source, although in some embodiments, the flow valve or additional flow valves can be used to control alternative fluid sources and/or an aspiration source. The docking branched manifold generally has a second branch having a hemostatic valve. The docking branched manifold has a tubular input at the distal end which includes a docking structure. The docking structure can pass through the hemostatic valve of the first branched manifold such that it can be positioned within the tubular segment of the first branched manifold.

The docking branched manifold generally can be used to flush the catheter using fluid from a fluid source, such as a syringe, pressurized vessel or a pump connected to a reservoir. The docking branched manifold can be equipped with a plurality of fluid sources, such as a contrast fluid source, a therapeutic agent fluid source, and/or a flush fluid source, such as buffered saline, although contrast fluid can also be used for flushing a clogged catheter. Also, aspiration can be delivered into the aspiration system from the docking branched manifold as an alternative or in addition to configuring aspiration to be delivered from the first fitting element, which may then optionally not include a manifold, such as shown in the figures described above. If the docking branched manifold is used to deliver a second fluid and/or aspiration as well as any further fluids, the docking branched manifold can comprise additional branches and/or additional branching along a second branch.

Generally, a control structure of the suction extension extending proximally can pass through a hemostatic valve with the valve closing around the control structure with an appropriate seal. Generally, the control structure can pass through both the hemostatic valve of the first branched manifold and the hemostatic valve of the second branched manifold so that it can be manipulated externally to the manifolds. The docking structure can slide over the control structure. In this configuration, the proximal end of the tubular extension can be drawn into a docked position with the docking structure. The docking structure may be configured to releaseably retain the tubular extension. For example, the docking structure may use an interference fit to secure the tubular extension. In embodiments, the docking structure can include a narrowing of the internal walls of the tubular input portion. For example, an interior surface of the tubular input may taper inwards until the interior diameter of the tubular input is less than the outer diameter of the tubular extension. In alternative or additional embodiments, the docking structure may include a flange on an interior surface of the tubular input. In embodiments, the docking structure may include a material on the interior surface of the tubular input configured to create a frictional fit to secure the tubular extension. In embodiments, the docking structure may include a structure on the interior surface of the tubular input configured to interface with a corresponding structure on an exterior surface of the tubular extension. For example, the docking structure may include a detent on the interior surface of the tubular input configured to interface with an indent on the exterior surface of the tubular extension.

With the tubular extension docked in the docking structure, the docking manifold may be disengaged from the first manifold. The docking branched manifold can be separated along with the suction extension by opening the hemostatic valve on the first fitting element, pulling the docking branched manifold away from the first fitting element, and resealing the first hemostatic valve when the tubular extension is clear of the valve. When the tubular extension is outside of the first fitting element, thrombus trapped within may be cleared out from the tubular extension. Opening the source valve attached to the docking branched manifold permits fluid to flow through the tubular extension. Fluid may flush the thrombus and any other debris or material trapped within the tubular extension. Once the tubular extension is clear it may be returned to the patient. It may be desirable to re-sterilize any components that have been exposed to the environment before reintroducing them into the patient, although generally the suction extension is maintained in a sterile condition outside from the patient so that it can be returned to the vasculature without further sterilization. To reintroduce the tubular extension, the first hemostatic valve of the first fitting element should be opened thereby permitting the tubular extension and docking structure to enter the first fitting element. With the docking structure in place within the first fitting element, the hemostatic valve may be tightened. The control structure may be used to move the tubular extension out of the docking structure, into the guide catheter, and back to a desired position within the patient. In some instances, aspiration may remain on while the tubular extension is cleared. In other instances, it may be preferable to halt aspiration when the tubular extension is not deployed in the guide catheter.

Following the completion of the revascularization of the vessel, the catheters are removed from the patient. Depending on the particular fittings used, several alternative procedures can be used to safely remove the catheters. If the fittings have an isolation section to remove the suction extension within the hemostatic seal, with the tubular extension safely parked external to the guide catheter, the procedure can be completed, which generally involves termination of suction and confirmation that the blockage is resolved. At the end of the procedure, the guide catheter can be safely removed from the patient. If the fittings do not include an isolation section, the suction extension may or may not be removed through a hemostatic valve prior to removal of the guide catheter. If the suction extension is not removed through a hemostatic valve to isolate it from the guide catheter, the distal end of the suction extension is generally located safely within the guide catheter lumen when the guide catheter is removed, and aspiration may be continued during at least a portion of the procedure involving the removal of the guide catheter.

In some embodiments, throughout the part of the procedure in which aspiration is applied, the pressure in the proximal fittings can be monitored. If the pressure in the proximal fittings remains within an expected range, the physician performing the procedure can proceed based on that knowledge. If the pressure increases, the physician can take appropriate actions, such as removing the suction extension from the patient, generally without the delivery of fluid through the tubular extension.

The devices and corresponding processes described herein provide improved functionality for performing therapeutic procedures for the removal of clots from vessels. As noted herein, the devices can be used in various combinations within medical systems for percutaneous procedures. Improved procedures provide additional safety measures while providing practical steps for performance by the medical professional handling the devices.

Aspiration Catheter System with Sliding Suction Extension/Aspiration Catheter

Aspiration catheter systems are described that take advantage of good suction available with a suction lumen having a larger proximal suction and a narrower diameter suction extension that uses the guide catheter lumen as a proximal suction lumen. A laterally slidable suction extension or aspiration catheter extends from a proximal section located within the guide lumen, and the suction extension/aspiration catheter can have a smaller distal diameter to provide access to narrow vessels while providing for delivery of other treatment structures and/or embolic protection structures as well as for a desirable level of suction for the removal of debris from the vessel. As noted above, suction extension and aspiration catheter terminology are used interchangeably. A control wire or other control structure can be attached to the suction extension to control sliding for providing selective lateral placement of the suction extension relative to a fixed guide catheter and a target treatment location. The suction extension/aspiration catheter generally has a connection section for forming a seal between the suction extension and the inner wall of the guide catheter. The connection section can have a fixed dimension or can be extendable radially.

In some embodiments, the suction extension comprises a connecting section that interfaces with the guide catheter lumen with a non-cylindrical cross section to provide for contact at two parts along the circumference. This non-cylindrical interface can block flow between the exterior of the proximal portion of the suction extension and proximal locations in the interior of the guide catheter while allowing relatively easy sliding of the suction extension relative to the guide catheter. A specific guide catheter design can incorporate various tubular elements along its shaft to provide for desired flexibility and a narrower diameter distal tubular element can be used to retain the proximal section of the suction extension within the guide catheter lumen. While fixed configuration connection sections can be effective, connection section designs based on radially extendable structures using mechanical frame that can be actuatable or self-extending with constraint release. Actuation of extension of a connection section can be achieved with an actuation cable that can be the same or distinct from the control structure. In some embodiments, actuation cable can comprise an electrical wire or, in other embodiments, a corewire and an overtube.

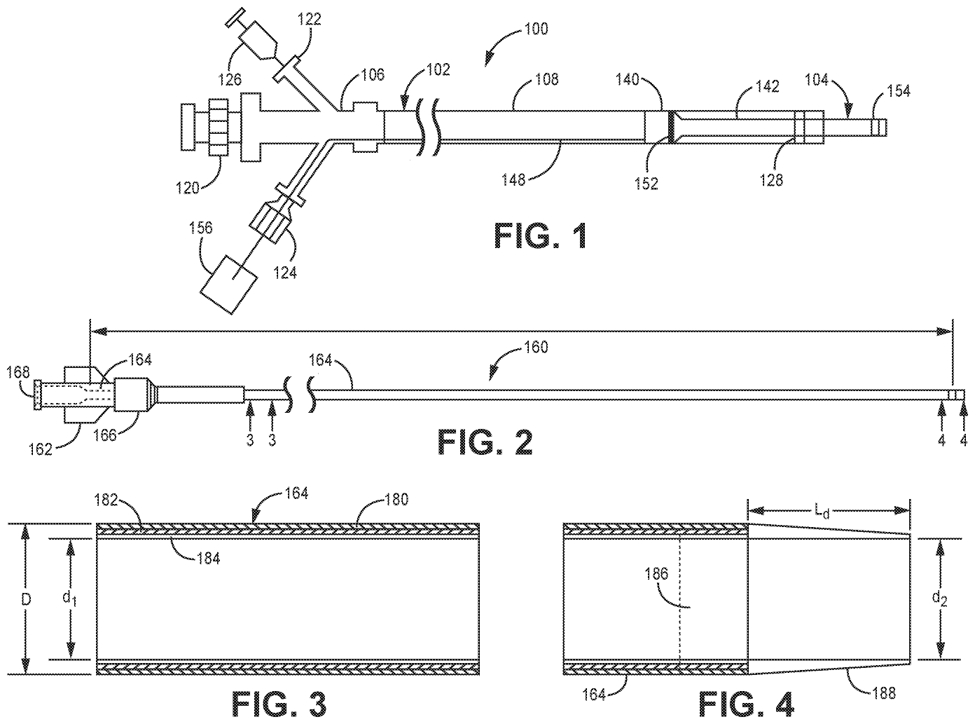

Referring to FIG. 1, suction system 100 comprises a suction adapted guide catheter 102 and a suction extension 104. The suction adapted guide catheter 102 comprises proximal section 106 and tubular shaft 108. Proximal section 106 generally is suitable for use also as a handle and generally can comprise a proximal fitting 120, a suction port 122 and an optional control wire port 124, as well as possibly other additional ports and/or fittings to provide desired functionality and access, in which all such ports and fittings can be arranged in a branch configuration or other suitable configuration. In general, proximal fitting 120 can comprise a suitable hemostatic valve, luer fitting or the like to provide for entry of a guidewire and/or structures delivered over the guidewire into the guide catheter lumen, such as alternative treatment structures and/or embolic protection devices.

In improved embodiments described herein, proximal fittings 120 can comprise a segment in which a tubular extension of suction extension 104 can be placed without extending into tubular shaft 108 of guide catheter 102 or through a hemostatic valve into the ambient environment. While desired features of fittings at the proximal end of the suction system 100 can be integral with proximal fitting 120, design flexibility can be achieved through embodiments of proximal fitting 120 comprising a connector, such as a Tuohy-Borst connector, and connection of fittings providing other desired features, such as a Y-branch, hemostatic valve, an extended tubular fitting to store the tubular extension of suction extension, etc. as fitting components that are attached for use to proximal fitting 120. Suitable fittings with additional functional features for incorporation with proximal fittings 120 are described in detail below in the treatment system section with an understanding that this disclosure below may be considered as integral portions of proximal fitting 120 rather than separate components.

For use with suction system 100, suitable embolic protection devices can be mounted on a guidewire, and/or other treatment structures can be used. Suitable treatment structures are described further below and can include, for example, fiber-based filters, stents, stent retrievers, atherectomy devices or the like. As shown in FIG. 1, a negative pressure device 126 is shown connected with suction port 122, and suitable negative pressure devices include, for example, syringes, pumps, such as peristaltic pumps, piston pumps or other suitable pumps, aspirator/venturi, or the like. Suitable pumps are available from Allied Healthcare Products, Inc., such as a Gomco™ brand pump, or a DRE DM-660™ pump.

In general, tubular shaft 108 can have an approximately constant diameter along its length, or some guide catheters can have sections with different diameters, generally with a smaller diameter section distal to a larger diameter section. In some embodiments described herein, a significant of the length of the tubular shaft has a constant diameter to make desired contact with a connecting section of the suction extension, which can be called an engagement section of the tubular shaft designed to engage the suction extension in a configuration suitable for the delivery of suction to a patient. Portions of the tubular shaft proximal to the engagement section can have a larger inner diameter and generally larger outer diameter relative to the engagement section. While a conventional guide catheter can be used in some embodiments for the suction catheter system, a specific design is described in detail below. A distal tubular portion of the tubular shaft can have a slightly narrower inner diameter to retain a portion of suction extension 104 within tubular shaft 108. Tubular shaft 108 can have one or more radiopaque marker bands to facilitate positioning of the tubular shaft within the patient as well as positioning the connecting section of the suction extension within the guide catheter lumen, and FIG. 1 shows a marker band 128 near the distal end of tubular shaft 108, although alternative positions can be used as desired. As described below, tubular shaft 108 can have coatings on the inner surface and/or the outer surface or portions thereof.

Suction extension 104 generally comprises a connecting section 140, tubular extension 142, and control structure 148, such as a control wire. All or a part of connecting section 140 can be configured to remain within the lumen of guide catheter 102. As shown in FIG. 1, connecting section 140 can comprise a radiopaque marker band 152, although connecting section may not have a marker band in some embodiments and in other embodiments can comprise a plurality of marker bands, and tubular extension 142 is shown with radiopaque marker band 154 near the distal tip of tubular extension 142, although again tubular extension 142 can comprise a plurality of radiopaque marker bands if desired. Control structure 148 can be a control wire or the like that connects with proximal portion 140 and in the assembled device extends exterior to the catheter, such as exiting through control wire port 124 or proximal fitting 120. Control structure 148 can be used to control positioning of connecting section 140 within the lumen of shaft 106. Control structure 148 can comprise a control tool 156, such as a handle, slide or other the like that can anchor a control wire or other connecting element to facilitate movement of the control wire. In some embodiments, alternative structures such as a plurality of wires or cylindrical wire assembly can connect the proximal portion to the proximal end of the suction catheter system to provide a desired level of control with respect to positioning the proximal section.

As noted above, the connecting section of suction extension engages the inner lumen of the guide catheter with an appropriate interface to reduce or eliminate flow of blood between the connecting section of the suction extension while allowing for the user to translate the suction extension relative to the guide catheter to position the tip of the tubular extension. A desirable design with a connecting section of the suction extension having a non-circular cross section has been found to particularly meet these criteria. With material selection as described herein, a very small average clearance can also be used between the connecting section of the suction extension and the interior of the guide catheter. When assembled, the inner lumen of the guide catheter can contact the connecting section of the suction extension at two locations around the circumference, which can provide partial rounding the cross section of the connecting section. This two location contact configuration provides desirable confinement of the flow while allowing for sliding of the suction extension by the user with appropriate ease.

The non-circular cross section of the connecting section (or a portion thereof) of the suction extension generally can be roughly oval in shape. While not intending to be limited by this term, in some embodiments, the cross section can have one axis of symmetry resembling the cross section of a conventional egg. As described below, the oval shape can be generated through the attachment of a wire control structure to the proximal section, although other structural features can be used to introduce the oval shape, such as with approximately one axis of symmetry or two axes of symmetry, although the oval can be asymmetric. Generally, the oval cross section can be partially characterized by a major axis, e.g., the longer dimension along an axis of symmetry, and a minor axis, e.g., the longest line segment connecting the circumference perpendicular to the major axis. While the specification of the major axis and the minor axis does not fully specify the oval since the specific shape is not specified, the major and minor axes can provide significant information regarding the dimensions and relative shape of the oval, especially since the shapes are generally not far out of a circular shape. Also, an average clearance can be defined using the largest value of the circumference (C) of the oval cross section and converting to an equivalent circle to define an approximate average diameter (Dₐ=C/π).

An embodiment of a guide catheter is shown in FIGS. 2-4. Referring to FIG. 2, guide catheter 160 comprises a connector fitted hub 162 with a portion of a Tuohy-Borst connector, luer connector or the like, shaft 164 and strain relief support 166. In this embodiment, the proximal end of shaft 164 passes through strain relief support 166 to connector fitted hub 162, and the components can be secured together with adhesive. Also, female connector 168 is located at the proximal end of connector fitted hub 162 for connection to a male connector fitting on a proximal fitting, such as a branched connector, which may have a rotating hemostatic valve with one or more branches.

A sectional view of a portion of shaft 164 near the proximal end is shown in FIG. 3. Referring to the embodiment of FIG. 3, shaft 164 comprises a polymer tube 180 with an embedded stainless steel wire braid 182 and a lubricious liner 184, e.g., polytetrafluoroethylene (PTFE) or other fluoropolymer. FIG. 4 shows the distal end of shaft 164. As shown in FIG. 4, a radiopaque marker band 186 is embedded in the polymer tubing near the distal end of shaft 164. Also, a distal section 188 of tubing is placed at the distal end of shaft 164 with a slightly reduced inner diameter, as explained further below. As shown in FIGS. 3 and 4, the metal braid ends adjacent marker band 186 (or overlaps with the marker band and terminates after), and distal section 188 is free of metal braiding in this embodiment. As described further below, the composition of the polymer tubing included in the shaft can vary along the length of shaft 164, for example, to increase flexibility of the shaft toward the distal end of the shaft. In some embodiments, different adjacent sections of polymer tubing can be heat bonded together and further supported with an overarching metal braiding and/or coil reinforcing the majority of the shaft. In some embodiments, the majority of the shaft 164 can have a constant inner diameter, except for distal section 188, to provide for the application of suction through the suction extension positioned at any location within the guide catheter proximal to distal section 188. But in alternative embodiments, a proximal section of shaft 164 can have a larger diameter if desired since the proximal section of the guide catheter may not be used for positioning the connecting section of the suction extension for the application of suction. Appropriate markers on the control wire can be used to ensure that the suction extension is positioned properly for the application of suction.

A lubricious coating, for example, a hydrophilic coating, can be placed on the outer surface of shaft 164 or a portion thereof. Suitable hydrophilic coatings include, for example, polyvinyl alcohol, heparin based coatings, .or the like. Hydrohylic coating solutions are commercially available, such as LUBRICENT® (Harland Medical Systems, MN, USA) or SERENE™ (Surmodics, Inc, MN, USA). Further description of the materials and manufacturing process are provided below.

The guide catheter can have an outer diameter (D) from about 5.5 Fr (1.667 mm diameter) to about 10 Fr (3.333 mm diameter), in further embodiments from about 6 Fr (1.833 mm diameter) to about 9 Fr (3 mm diameter), and in some embodiments from about 6.25 Fr (2 mm diameter) to about 8.5Fr (2.833 mm diameter). The guide catheter measurement are generally referenced to the outer diameter, and the inner diameter is less than the outer diameter by twice the wall thickness. In general, the inner diameter of the main portion of shaft 164 (d₁) can range from about 0.8 mm to about 3.175 mm, in further embodiments from about 0.9 mm to about 2.85 mm and in additional embodiments from about 1.00 mm to about 2.7 mm. The reduction in inner diameter of distal section 188 (d₂) relative to the inner diameter of an engagement section of shaft 164 (d₁) can be from about 0.034 mm (0.00134 in) to about 0.25 mm (0.0098 in) and in further embodiments from about 0.05 mm (0.002 in) to about 0.20 mm (0.0079 in). The length of the guide catheter shaft can be from about 30 cm to about 150 cm, in further embodiments from about 35 cm to about 130 cm and in additional embodiments from about 40 cm to about 120 cm, and is generally selected to be suitable for the corresponding procedure. In some embodiments, distal section 188 can have a length (L_{d}) from about 1 mm to about 50 mm, in further embodiments from about 1.5 mm to about 25 mm, and in other embodiments from about 2 mm to about 20 mm. A person of ordinary skill in the art will recognize that additional ranges of dimensions within the explicit ranges above are contemplated and are within the present disclosure.

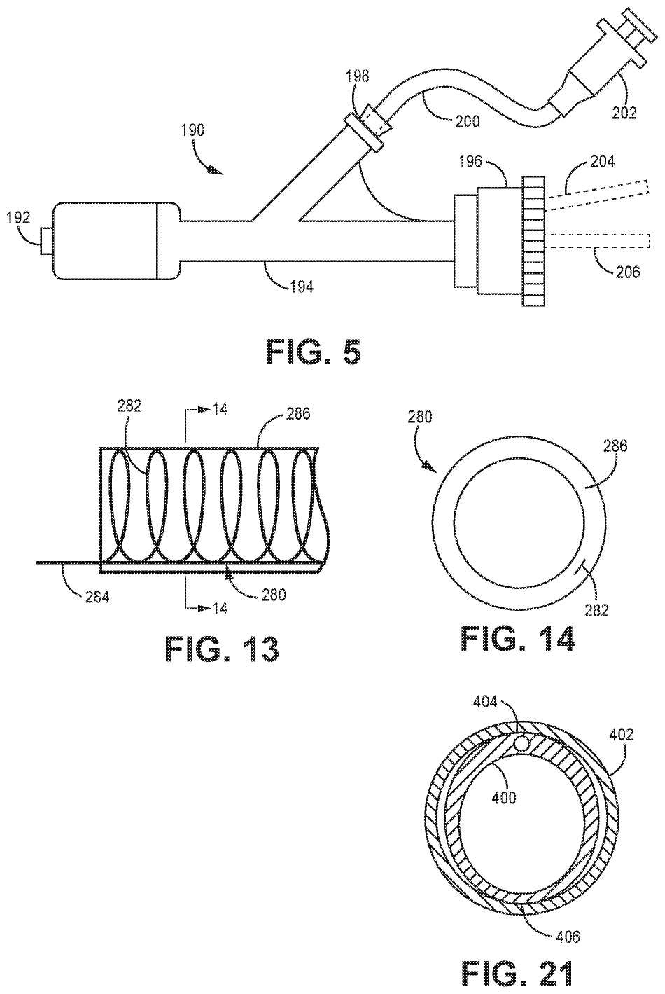

For use of the guide catheter of FIG. 2 to form analogous proximal fittings of FIG. 1, a Y-branch hemostatic valve connector 190 can be used, such as the embodiment shown in FIG. 5. Y-branch hemostatic valve connector 190 comprises a male connector 192, a Y-branch frame 194 with branching flow channels, rotating hemostatic valve 196, connector 198, tubing 200 connected to Y-branch frame 194 at connector 198, and suction device 202 connected to tubing 200. Male connector element 192 can be attached to female connector element 168 of FIG. 2. As schematically shown in FIG. 5, a control wire 204 and a guidewire 206 are both shown exiting hemostatic valve 196, and guidewire 206 can be used to guide therapeutic devices through a guide catheter through the hemostatic valve. Various branched hemostatic valve connectors are available from commercial suppliers, such as Merit Medical, UT, USA. More generally, a range of fittings can be attached to connector fitted hub 162 of guide catheter 160, and improved embodiments of fittings with a portion for placement of the tubular extension of the suction extension are described in more detail under the treatment systems section below.

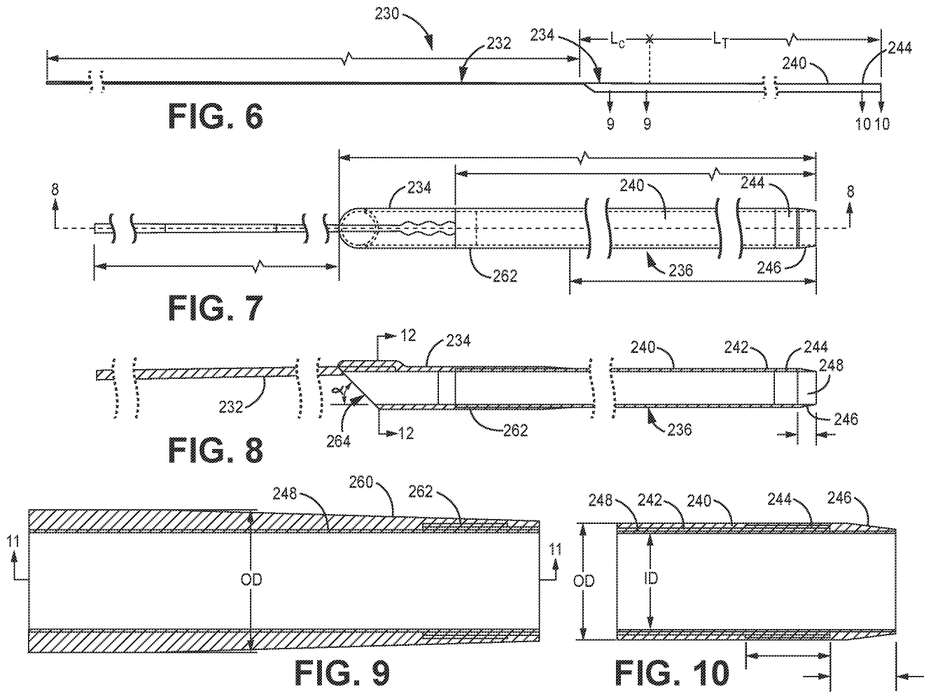

An embodiment of a suction extension is shown in FIGS. 6-12. Referring to FIG. 6, suction extension 230 comprises a control wire 232, connecting section 234 and tubular extension 236. Connecting section 234 connects with control wire 232, which extends in a proximal direction from the connecting section, and tubular extension 236, which extends in a distal direction from the connecting section. In general, control wire 232 can be a solid wire, coil or the like that provides for transmission of pulling and pushing forces to connecting section 234, which correspondingly can move with the tubular extension 236 relative to a guide catheter in the assembled suction catheter system. Control wire 232 can have any reasonable cross sectional shape, which can be different at different locations along the length of the control wire. Also, the control wire can be tapered to a smaller circumference toward the distal end of the control wire. Generally, control wire 232 is made of biocompatible metal, such as stainless steel, titanium or the like, although other materials that have appropriate balance of rigidity and flexibility can be used in principle. In some embodiments, the control wire is a round metal wire with an average diameter along its length from about 0.010 inches (0.254 mm) to about 0.040 inches (1.01 mm) and in further embodiments from about 0.0125 inches (0.32 mm) to about 0.030 inches (0.76 mm). The length of control wire 232 is generally somewhat longer than the guide catheter so that the guide wire extends from the proximal end of the guide catheter, such as 5 cm or more longer than the guide catheter. A person or ordinary skill in the art will recognize that additional ranges within the explicit dimensional ranges above are contemplated and are within the present disclosure.

Connecting section 234 generally is distinguishable by a larger outer diameter than tubular extension 236, and tubular extension 236 extends from the connecting section 234 in a distal direction. In the embodiment of FIGS. 6-12, tubular extension 236 has an approximately constant outer and inner diameter, and a further embodiment is described below with a step down diameter along the tubular extension. Referring to a sectional view in FIG. 10, tubular extension comprises a polymer tube 240, a metal coil reinforcement 242 and a radiopaque marker band 244. Metal coil reinforcement 242 can comprise a flat metal wire, which can extend in some embodiments from roughly radiopaque marker band 244 to a radiopaque marker band in connecting section 234, described further below, although the metal coil reinforcement can extend over the marker bands. Polymer tube 240 can remain the same along the length of tubular extension 236, or the polymer can be changed as different positions along tubular extension 236, for example, getting more flexible in a distal direction. Different sections of polymer can be heat bonded during construction, and metal coil reinforcement 242 as well as optionally a polymer overlayer can further stabilize connected sections of polymer tubing. A tip 246 of tubular extension 236 distal to radiopaque marker band 244 can comprise polymer tubing 240 free of metal reinforcement. A low friction liner 248, such as PTFE or other fluoropolymer, can extend along the length of tubular extension 236 and/or connecting section 234, or portions thereof.

The relationship of connecting section 234 with control wire 232 and tubular extension 236 are shown in FIGS. 6-8. Sectional views of portions of connecting section 234 are shown in FIGS. 9, 11 and 12 and show certain details of the structure. Connecting section 234 can comprise polymer tubing 260 and radiopaque marker band 262. Polymer tubing 260 has a proximal opening 264 that can be angled relative to a longitudinal axis of the polymer tubing to facilitate delivery of devices through the suction extension, although a right angle can be used if desired. The angle α is marked on FIG. 8 and can range from 25 degrees to about 85 degrees, in further embodiments from about 30 degrees to about 80 degrees, and in additional embodiments from about 33 degrees to about 75 degrees. A person of ordinary skill in the art will recognize that additional ranges of angles within the ranges above are contemplated and are in the present disclosure.

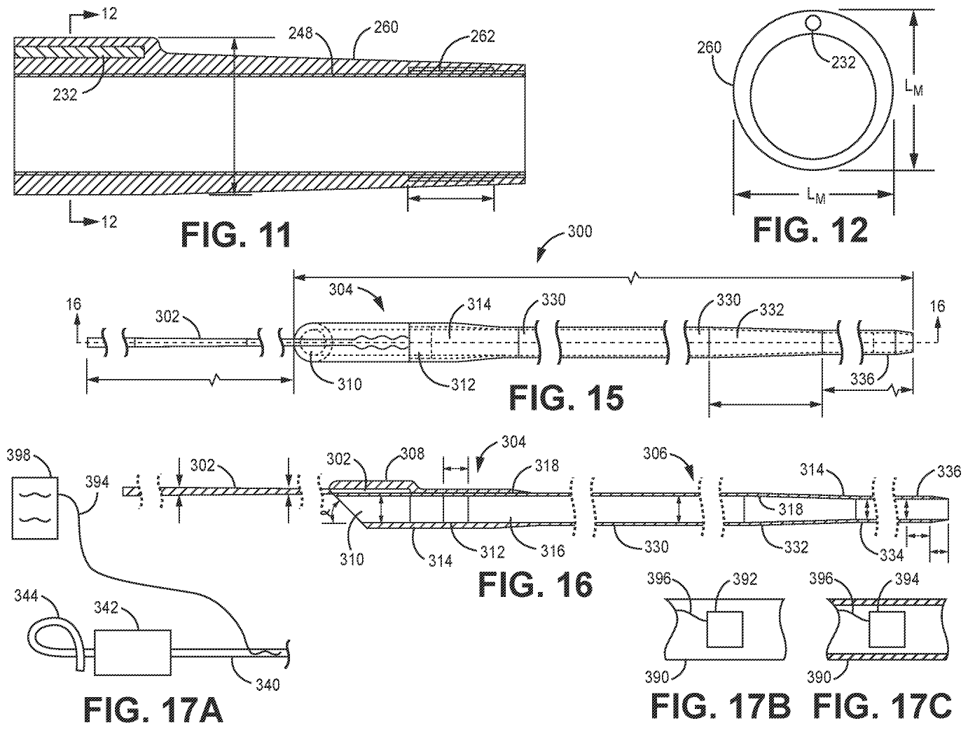

The interface of control wire 232 with connecting section 234 can serve the purpose of both securing the components together as well as helping to form the shape of connecting section 234, which can be selected to provide a desired interface with the interior of the guide catheter lumen. Specifically, the connection of the control wire with the connecting section can facilitate the formation of the oval cross section of the connecting section. In alternative embodiments, control wire 232 can terminate with a flat wire coil that is embedded into a polymer tube to substantially maintain the shape of the connecting section, as described in the '938 application and below. In additional or alternative embodiments, an oval shape of the connecting section can be introduced through the molding or other shaping of the polymer which may or may not be combined with a bump due to an embedded control wire. Suitable dimensions of the oval cross section and the processing to form the connecting section are described further below. Low friction liner 248 can extend through the inner lumen of connecting section 234, as shown in FIGS. 9 and 11, or in some embodiments a separate low friction liner can be included in connecting section 234 if desired.

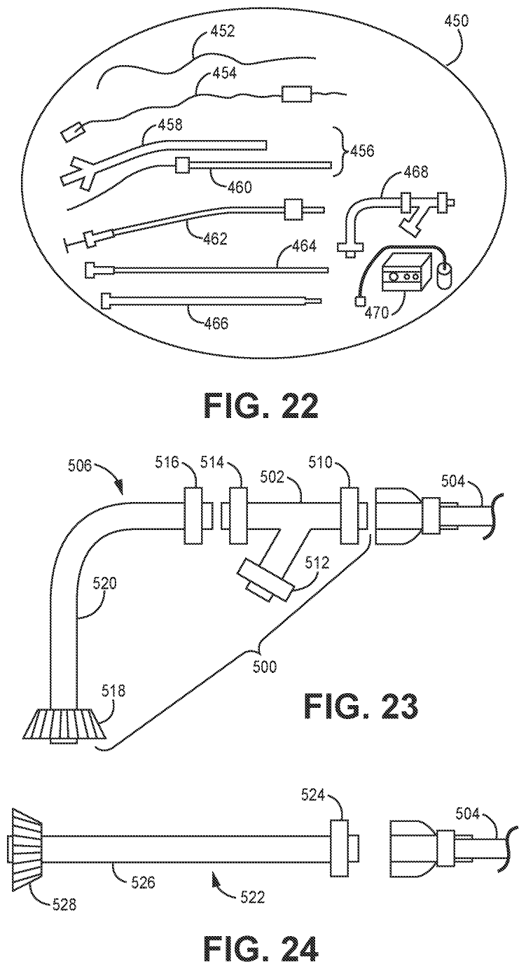

Referring to FIGS. 8, 11 and 12, the distal end of control wire 232 is embedded in polymer associated with polymer tubing 260. Supplementing the polymer wall to secure control wire 232 alters the cross sectional shape that results in a major axis (LM) greater than the minor axis (Lm), as can be seen clearly in FIG. 12. As noted above, the non-circular cross section is advantageous for the interface of the suction extension with the guide catheter. The cross section of an alternative embodiment of a connecting section 280 with a non-circular shape is shown in FIGS. 13 and 14. In this embodiment, a flattened metal coil 282 at the end of a control wire 284 is embedded in a polymer tube 286 with a noncircular cross section. The non-circular cross section is formed in this embodiment through forming the polymer with a thicker wall along one edge of the circumference, as can be seen in the sectional view of FIG. 14. A corresponding circular embodiment is shown in FIGS. 21 and 22 of the '938 application. The connecting section may or may not have an approximately constant outer diameter over its length, and the outer diameter may taper, e.g. a gradual taper, step-wise taper or combination thereof, over at least a portion of its length to roughly the outer diameter of the adjacent section of the tubular extension.

In some embodiments, the proximal end of connecting section is suitable for docking in a docking element of a fitting element to provide for removal of the suction extension from hemostatic isolation in association with the fitting element. Such a fitting docked with the suction extension can be used to clear clots from the suction extension in the docked position. Once cleared of clots, the suction extension can be reintroduced into the patient for further use to remove additional thrombus from the patient's vessel. Suitable fittings are described in detail below.

An alternative embodiment of a suction extension is shown in FIGS. 15 and 16. Suction extension 300 comprises control wire 302, connecting section 304 and tubular extension 306. Control wire 302 and connecting section 304 can be analogous to control wire 232 and connecting section 234, respectively, for the embodiment of FIGS. 6-12. Referring to FIG. 16, the distal end of control wire 302 is embedded in polymer within connecting section 304 forming a distension 308 along a surface of connecting section 304. A proximal opening 310 into the lumen of connecting section 304 forms an angle α with respect to the axis of connecting section 304. Connecting section 304 comprises a radiopaque marker band 312. The body of connecting section 304 is a polymer tube 314. Low friction liner 316, such as PTFE or other fluoropolymer, can extend along the lumen of connecting section 304 and/or tubular extension 306 or selected fractions thereof. Metal reinforcement, such as a flat metal wire coil, can reinforce polymer tube 314 or a fraction thereof. As shown in FIG. 16, flat metal wire coil 318 is embedded through the polymer tube 314 distal to radiopaque marker band 312 and extending to tubular extension 306. Furthermore, the asymmetric cross section shown in FIGS. 12 and 14 as well as the control wire attachment approaches of FIGS. 11 and 13 can apply also to the embodiment of FIGS. 15 and 16.

Referring to FIGS. 15 and 16, tubular extension 306 comprises a first tubular section 330, taper section 332 and second tubular section 334 having a smaller diameter than first tubular section 330. Taper section 332 tapers between the diameter of first tubular section 330 and the diameter of the second tubular section 334. Second tubular section 334 comprises a radiopaque marker band 336. Flat metal wire coil 318 extends from radiopaque marker band 336 to radiopaque marker band 312 within connecting section 304, embedded within a polymer tube. The end of second tubular section 334 distal to radiopaque marker band 336 can be free of metal reinforcement. As noted above, a low friction liner 316 can extend along lumen wall for the length of tubular extension 306 or a selected fraction thereof. The body of the first tubular section 330, taper section 332 and second tubular section 334 generally comprises a thermoplastic polymer tube. Sections of polymer tube can be heat bonded together and further supported by the embedded flat metal wire coil 318, optionally with heat shrink polymer film or the like covering the metal reinforcement. The composition of the polymer tube can vary along the length as desired to select a particular flexibility, generally more flexible toward the distal end of the device, and the polymer composition can be varied for the different section 330, 332, 334 and/or within the sections.

As shown in FIGS. 15 and 16, taper section 332 provides an approximately linear transition of diameters from the wider diameter of first tubular section 330 to the narrower diameter of second tubular section 334. In alternative embodiments, a taper section can have nonlinear changes in diameter if desired, but the change is generally monotonic. The taper section can be formed through an extrusion process or through conforming of a thermoplastic polymer to a mandrel shape or other suitable process approach known in the art.

A significant aspect of the suction extension is the narrower diameter suction tip relative to the guide catheter, and the step down diameter of the second tubular section of the embodiment of FIGS. 15 and 16 allow for further reach into narrow neurovascular vessels. The effective suction lumen then extends through the guide catheter into the connecting section of the suction extension and then into the tubular extension, which can have further step downs in diameter. The inner diameter of the connecting section may or may not be the same as the inner diameter of the first tubular section. The narrow diameter of the tubular extension provides for reach into small circuitous blood vessels and the use of the larger diameter proximal suction lumen improves the suction performance significantly without detracting from the ability to reach appropriate locations.

FIG. 17 shows an alternative embodiment of the suction extension in which the control structure has a handle at or near its proximal end. Referring to FIG. 17, control structure/wire 340 has a handle 342 secured near its proximal end. Handle 342 may or may not comprises structure to provide for disengagement of the handle. A specific embodiment of a handle is described in detail below. Control structure/wire 340 has a twist 344 at its distal end to inhibit the removal of handle 342 from control structure 340. Twist 344 can refer to or be replaced with a bend, a knot, an anchor, or other structure or distortion that prevents or inhibits the removal of handle 342 from control structure 340.

To facilitate monitoring of the pressures and flows through the aspiration system, various components of the aspiration system can be instrumented with sensors. Referring to FIGS. 17B and 17C, tubular shaft 390 can be provided with one or more pressure sensors. Pressure sensor 392 may be positioned to measure the pressure exterior to tubular shaft 390, such as within a vessel. Pressure sensor 394 is positioned to measure the pressure within tubular shaft 390. In embodiments, pressure sensor 392,394 may be positioned at or near a distal tip of the tubular shaft. In embodiments, sensor 392,394 may be positioned anywhere along the tubular shaft. In some embodiments, sensor 392,394 may be strategically positioned to have an acceptable impact upon the flexibility of the tubular shaft as it is advanced through the vasculature.

Wiring for the sensors can be embedded within a polymer wall or otherwise tracked along the length of the tubular shaft to a location near its proximal end. For example, as shown in FIGS. 17A-C, wire 396 connects to pressure sensor 142, 144 and controller/display 398. The pressure readings could also be transmitted wirelessly. Wireless pressure measurements are used in the Pressurewire™ X guidewire from Abbott. In some embodiments, wire 394, for example, may terminate at a transceiver that is in wireless communication with controller/display 398. Various suitable pressure sensors can be adapted for use in these devices. Integrated circuit pressure sensors can be used such as the Infineon KP236 pressure sensor. A piezoresistive pressure die P330 W is available from Nova®Sensor with a thickness of 120 microns. These sensors can be appropriately embedded into the wall of the catheter to secure the sensors with appropriate modification of the catheter wall. The use of these pressure sensors in a catheter is described in published U.S. patent application 2018/0010974A to Bueche et al., entitled “Pressure Sensor System,” incorporated herein by reference.

To further provide for suction strength, the tubular extension itself can have different sections with stepped down diameters, such as shown in the embodiment of FIGS. 15 and 16. In general, the arteries progressively decrease in diameter so a section with a somewhat larger diameter may be desirable consistent with the reach of the suction tip into a selected narrow vessel. With respect to first tubular section, this section generally has an approximately constant diameter (generally inner diameter or outer diameter with an assumption of approximately constant wall thickness) that is generally from about 0.95D to about [d+0.1(D−d)], in further embodiments from about 0.925D to about [d+0.25(D−d)], and in some embodiments from about 0.9D to about [d+0.35(D−d)], where d is the diameter of second tubular section and D is the average diameter of the connecting section. The length of first tubular section can be from about 10% to about 90%, in further embodiments from about 20% to about 80% and in additional embodiments form about 30% to about 70% of the total length of tubular extension, e.g., the total length of first tubular section, second tubular section, and the optional transition section or just a single tubular section for corresponding embodiments (L_{T} in FIG. 6). The connecting section can have a length (L_{C} in FIG. 6) from about 4 mm to about 8 cm and in further embodiments from about 5 mm to about 6 cm. A person of ordinary skill in the art will recognize that additional ranges of dimensions and relative dimensions within the explicit ranges above are contemplated and are within the present disclosure. While FIGS. 15 and 16 show a tubular extension with one step down in diameter to a second tubular section, in other embodiments there can be additional constant diameter tubular sections further stepping down the diameter, which further divide the length of the entire tubular extension specified above. For example, there can be a further intermediate tubular section, two further intermediate tubular sections or more than two further intermediate tubular sections.

The tubular extension or distal tubular section of the tubular extension for embodiments with a plurality of tubular sections with different inner diameters can have an inner diameter from about 20 percent to about 90 percent of the inner diameter of the engagement section of the guide catheter, and in further embodiments from about 30 percent to about 85 percent and in additional embodiments from about 35 percent to about 80 percent of the inner diameter of the engagement section of the tubular shaft. For example, the distal tip of the tubular extension can have an inner diameter in a range from about 0.5 mm to about 1.9 mm, in further embodiments from about 0.6 mm to about 1.8 mm, and in other embodiments from about 0.65 mm to about 1.75 mm. The tubular extension can have a length from about 3 cm to about 60 cm, in some embodiments from about 5 cm to about 55 cm and in further embodiments from about 8 cm to about 50 cm. A person of ordinary skill in the art will recognize that additional ranges of dimensions within the explicit ranges above are contemplated and are within the present disclosure.

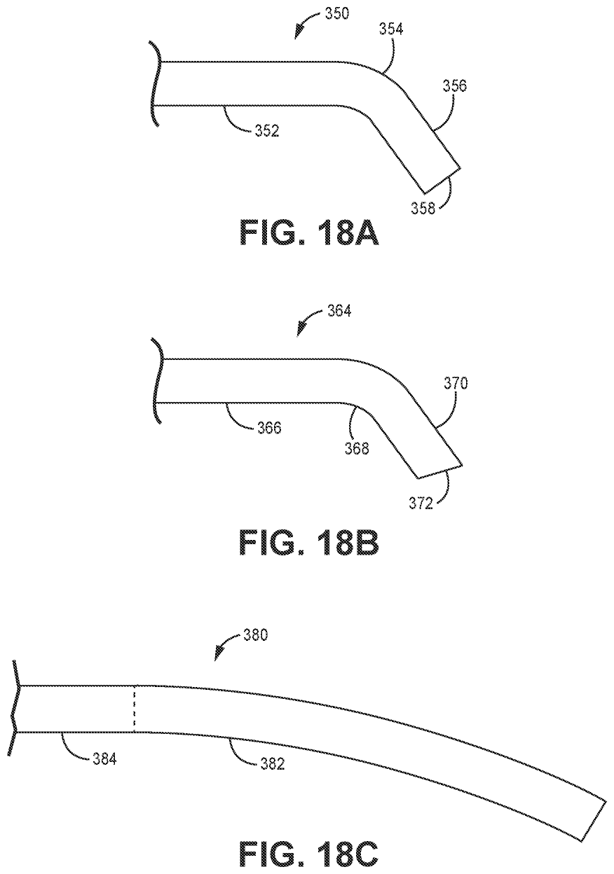

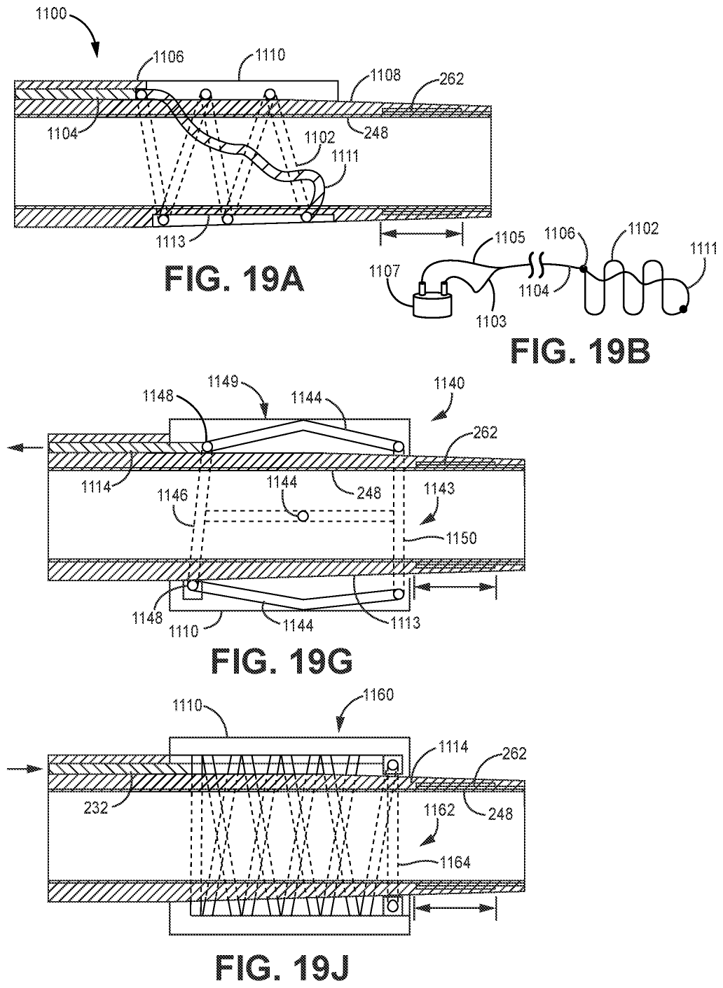

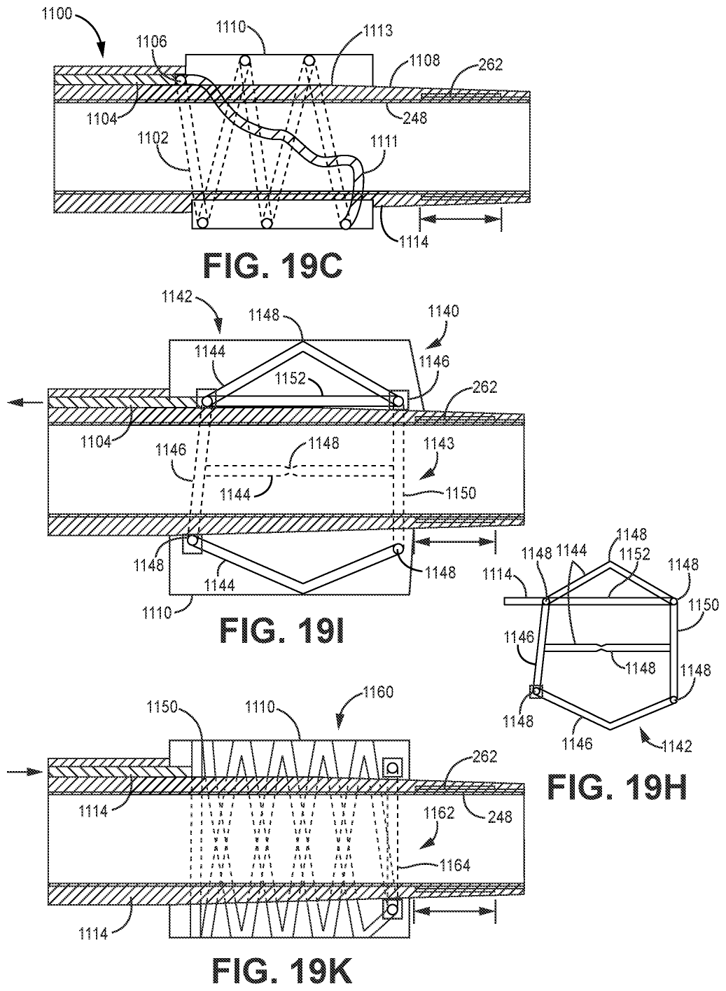

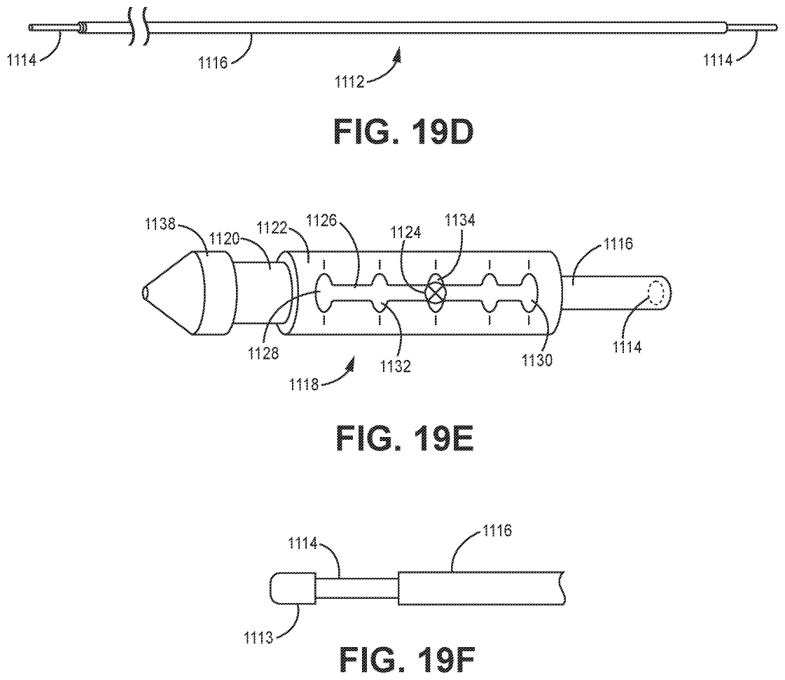

The distal tip of the tubular extension can be bent or curved in its natural unstressed configuration. It has been found generally that a bent tip catheter can facilitate tracking of the catheter over a guidewire without adversely altering the suction abilities. See, for example, U.S. Pat. No. 8,021,351 to Boldenow et al., entitled “Tracking Aspiration Catheter,” incorporated herein by reference. Two general versions of a bent suction tip are shown in FIGS. 18 and 19. Referring to FIG. 18, suction tip 350 comprises a straight section 352, bend 354 and bent tip section 356 with a flat distal opening 358 approximately perpendicular to the axis of bent tip section 356. Referring to FIG. 19, suction tip 364 comprises a straight section 366, bend 368 and bent tip section 370 with an angled distal opening 372 at a non-perpendicular angle to the axis of bent tip section 370. Bent tip sections 356, 370 are generally cylindrical and can have approximately the same diameters as corresponding straight sections 352, 366. While two shapes of openings are shown in FIGS. 18A and 18B, any reasonable shape of the opening generally can be used.

A specific embodiment of a bent tip for a suction extension 380 is shown in FIG. 18C. In this embodiment, the distal tip 382 is curved with no straight section at the distal end in this embodiment, although alternative embodiments can have short straight segment at the distal end. Distal tip 382 extends from a straight section 384 of suction extension 380. The arc of the curve is approximately circular, but other gentle arcs can be used, in which case the radius of curvature can be an average over the arc.

In this embodiment, the curvature of the tip is gradual so that the distal tip may not have a straight section. An angle γ can be defined based on the point of initial curvature and the natural position of the tip taken at the middle of the distal opening. In some embodiments, angle γ can be from about 5 degrees to about 21 degrees and in further embodiment from about 7 degrees to about degrees. To achieve the gentle curvature, the radius of curvature generally is relatively large, and in some embodiments, the radius of curvature can be from about 21 mm to about 100 mm and in further embodiments from about 25 mm to about 75 mm. In some embodiments, a straight portion of the tip after the curve can have a length no more than about 1 cm, and in other embodiments from about 0.1 mm to about 6 mm and in further embodiments from about 0.5 mm to about 4 mm. In alternative embodiments, the curve consists of a gradual arc with no significant straight section distal to it, such that the curve or bend is specified by the angle and radius of curvature. A person of ordinary skill in the art will recognize that additional ranges of angles, radii and lengths within the explicit ranges above are contemplated and are within the present disclosure.

As noted above, the connecting section of the suction extension can have a non-circular, oval cross section, which can interface then with the inner surface in the lumen of the guide catheter to contact the inner surface at two locations along the circumference. The interface between the connecting section of the suction extension and the engagement section of the guide catheter reduces or eliminates any flow between surfaces so that essentially all of the suction flow passes through the lumen of the suction extension. At the same time, the suction extension can be positioned longitudinally within the engagement section to position the suction extension by a user through sliding the control structure. These various conditions can be balanced effectively to provide the desired functionality.

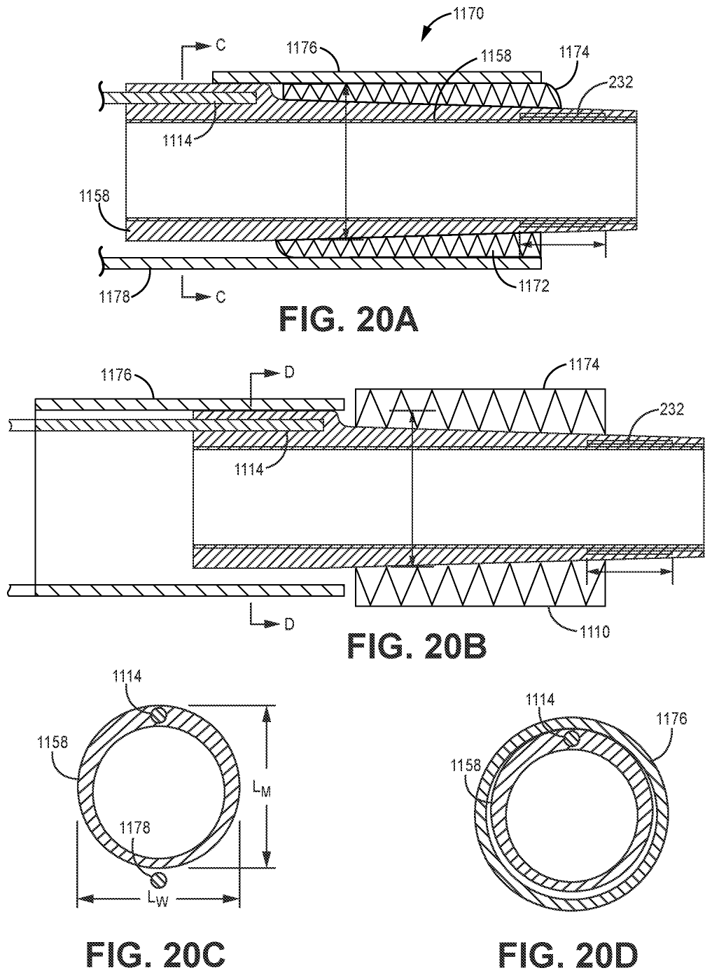

While a fixed connecting section, such as connecting section 234 in FIG. 11, can provide desirable functionality in a practical device with suitable sliding ability while presenting little or no blood flow between the connecting section and the guide catheter wall, it can be desirable to have an extendable section that can transition between a disengaged configuration and an engaged configuration. Such a structure can offer the promise of an easier to slide aspiration catheter when the connecting section is disengaged while still providing an appropriate seal following engagement of the connecting section. Alternative extendable connection sections with actuatable engagement mechanisms are described in FIGS. 19A-K, and self-extending connection sections are described in FIGS. 20A-D. As described in FIGS. 19A-K, the control wire provides the actuation mechanism to deploy the engagement component of the connection section. These actuatable embodiments generally may provide for disengagement and reengagement, as desired, although disengagement may or may not return the connection section back to the un-deployed configuration. In these embodiments, a framework functions to perform the deployment or un-deployment while a cover over the framework provides the seal when the framework holds the cover to the inner wall of the guide catheter. The cover generally is sealed around the connecting along the respective edges at either end of the frame along the axial extent of the connecting section.

Referring to FIGS. 19A-C, a sectional side view of suction extension 1100 with a connecting section 1108 with a thermal actuator 1102 is shown. Control wire 1104 may comprise electrical connections to thermal actuator 1102, such that it also functions as the actuation cable. At its proximal end, control wire 1104 has two wires 1103, 1105 separated for connection to a current source 1107, which can be a battery, transformer or the like to supply direct current, or to a source of alternating current. Thermal actuator 1102 may comprise a coil or a stent like structure positioned around a tubular feature 1113 of connecting section 1108 such that thermal actuator 1102 can deform to obtain an extended diameter structure, and the structure obtains a larger diameter as it is heated due to resistive heating. Conversely, thermal actuator 1102 may contract or soften as it cools. Accordingly, the expansion and subsequent retraction of thermal actuator 1102 may be controlled through the addition and removal of heat, such as resistive heating supplied by applying an electrical current to control wire 1104 and cooling resulting from thermal dissipation following the discontinuation of the electrical current. Cover 1110 is located around thermal actuator 1102 to provide a seal when thermal actuator is in its extended configuration.

Control wire can comprise two conductive metal wires, such as copper or silver, separated and insulated by polymer or other electrically insulating material. Metal alloys can be used for providing resistive heating, such as nickel chromium alloys or other materials known in the art. But other alloys provide shape memory characteristics as well as being suitable for resistive heating. Thus, thermal actuator 1102 can be formed from nickel titanium ally (NITINOL) shape memory metal that provides for both resistive heating and thermal shape changes. The Nitinol element can be formed with a shape memory of the extended configuration and then placed in the reduced diameter configuration for delivery. In some embodiments, the coil of thermal actuator 1102 can comprise the resistive heating alloy within a polymer coating, although it may be desirable to not coat the alloy to provide for faster cooling upon discontinuing electrical current. Thermal actuator 1102 can be connected to one of the conductive wires of control wire 1104 at connection 1106. Conductive wire 1111 connects control wire 1104 with the far end of thermal actuator 1102, in a configuration to insulate conduction with other parts of the structure, to provide the conduction circuit through the structure. The transition can be selected to take place between 40° C. and 80° C. The use of a thermally actuated NITINOL stent is described in published U.S. patent application 2001/0049549 to Boylan et al., entitled “Marker Device for Rotationally Orienting a Stent Delivery System Prior to Deploying a Curved Self-Expanding Stent,” incorporated herein by reference. Controlled deployment and engineering of actuation temperatures for a Nitinol medical device frame is described in published U.S. patent application 2019/0076275 to Kim, entitled “Endovascular Device Configured for Sequenced Shape Memory Deployment in a Body Vessel,” incorporated herein by reference. The use of Nitinol for resistive heating is discussed in U.S. Pat. No. 6,410,886 to Julien, entitled “Nitinol Heater Elements,” incorporated herein by reference. Upon cooling of the Nitinol below its transition temperature, the Nitinol softens, which can allow movement of connection section 1108 even if the thermal actuator does not return to it un-deployed structure.

When thermal actuator 1102 expands its diameter, it can engage the catheter wall. Generally, thermal actuator 1102 is further surrounded by a cover 1110. The cover can be, for example an elastomeric polymer sheet, a woven fabric, nanofiber spun structure, expanded PTFE, combinations thereof, or the like. Cover 1110 can be water repellant or at least resistive to flow of blood to form a sufficient seal for the deployed connection section. Radiopaque band 262 may further assist with positioning connecting section 1100 within the vasculature.

Referring to FIG. 19D, a control wire 1112 is shown comprising a core wire 1114 within an overtube 1116. As described in detail below, sliding core wire 1114 laterally within overtube 1116 may actuate an engagement element in the connecting section. Corewire 1114 is longer than overtube 1116 with the corewire extending from the proximal end of the overtube in all functional configurations to provide control of the relative position of the corewire, and corewire 1114 extends from the distal end of the overtube. The cross section of the overtube can be characterized by an inner diameter and an outer diameter. The inner diameter general ranges from about 0.001 inches to about 0.03 inches, in further embodiment from about 0.003 inches to about 0.02 inches and in additional embodiments from about 0.005 inches to about 0.01 inches. The outer diameter generally ranges from about 0.005 inches to about 0.04 inches, in further embodiments from about 0.007 inches to about 0.03 inches, in additional embodiments from about 0.008 inches to about 0.02 inches and in other embodiments from about 0.009 inches to about 0.015 inches, with standard guidewire outer diameters being about 0.010 inches to 0.014 inches. The corewire generally has a diameter just slightly less than the inner diameter of the tube by about 0.001 inches to about 0.003 inches. A person of ordinary skill in the art will recognize that additional ranges within the explicit ranges for the diameters are contemplated and are within the present disclosure.

In general, corewire 1114 and overtube 1116 can be formed from one or more of various materials, such as polymers, metals and combinations thereof. The overtube and corewire may or may not be formed from the same material. Suitable materials are generally biocompatible in that they are non-toxic, non-carcinogenic and blood compatible and do not induce hemolysis or a significant immunological response. Suitable biocompatible metals include, for example, titanium, cobalt, stainless steel, nickel, iron alloys, cobalt alloys, such as Elgiloy®, a cobalt-chromium-nickel alloy, MP35N, a nickel-cobalt-chromium-molybdenum alloy, Nitinol, a nickel-titanium alloy or a combination thereof.

Suitable polymers include, for example, synthetic polymers as well as purified biological polymers and combinations thereof. Suitable synthetic polymers include, for example, polyamides (e.g., nylon), polyesters (e.g., polyethylene teraphthalate), polyacetals/polyketals, polyimide, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Based on desirable properties and experience in the medical device field, suitable synthetic polymers include, in particular, polyether ether ketones, polyacetals, polyamides (e.g., nylons), polyurethanes, polytetrafluoroethylene, polyester teraphthalate, polycarbonates, polysulfone and copolymers and mixtures thereof.

In some embodiments, the surface of the corewire, the inner surface of the overtube, the outer surface of the overtube, portions thereof or combinations thereof is coated with a friction reducing agent. Suitable friction reducing agents include, for example, suitable polymers, such as polytetrafluorethylene, i.e., Teflon® or a polymer coating such as parylene. The coating of the corewire or a portion thereof can facilitate relative longitudinal motion of the corewire relative to the overtube.

Various tools may be used to facilitate change the relative longitudinal position of the corewire and overtube. FIG. 19E illustrates an embodiment of an actuation tool with a ratchet style pull element 1118. Pull element 1118 comprises slide 1120 and body 1122. Button 1124 is attached to slide 1120. Body 1122 has a slot 1126 that constrains the position of the button 1124. In this embodiment, slot 1126 has two end stop points 1128, 1130 and three intermediate stop points 1132, 1134, 1136. Markings can label the particular stop points. The operator can select the particular stop point to position corewire 1114 at one of the end points of its range of motion or at an intermediate stop point relative to overtube 1116. In some embodiments, the selection of a particular stop point can correspond to a selected guide catheter size. Slide 1120 releasably secures to corewire 1114, for example, with a tightening collet 1138 or other suitable releasable attachment device. Similarly, body 1122 can releasably secure to overtube 1116, for example, through the tightening of a screw or other suitable releasable fastener. Friction based ratchet structures can be similarly used. In other embodiments, one interment stop point, two intermediate stop points, four intermediate stop points, five intermediate stop points, ten intermediate stop points, twenty intermediate stop points, or any number in between can be used as an alternative to the three intermediate stop points shown in FIG. 19D based on the disclosure herein.

Referring to an embodiment in FIG. 19F, a handle 1113 is secured to the proximal end of corewire 1114. Handle 1113 can be used to grip corewire 1114 to effectuate relative motion of corewire 1114 and overtube 1116. Pushing or pulling handle 1113 causes the distal end of corewire 1114 to move laterally. As discussing below, lateral movements of corewire 1114 can be used to actuate or de-actuate other structures connected to the corewire.

Referring to FIGS. 19G-I, a sectional side view of a connecting section 1140 with a manually activated seal 1142 is shown that uses an actuation control wire as shown in FIGS. 19D-F. In this embodiment, corewire 1114 is pulled relative to overtube 1116 to actuate manually actuated seal 1142 to form the seal, and pushing corewire 1114 disengages the seal. Manually activated seal 1142 comprises a frame structure 1143 surrounding tubular feature 1113 of connecting section 1140, in which manually actuated seal 1142 comprises a plurality of foldable axial ribs 1144 and rigid proximal end pieces 1146. As shown in FIGS. 19G-I, four foldable axial ribs 1144 are used, but a larger number can be used if more effective to form a desired seal. Rigid end pieces 1146 can be held in place by tight wrapping about tubular element 1145, embedding in a polymer collar, combinations thereof, or other suitable anchoring structure. Axial ribs 1144 and proximal end pieces 1146 are joined at pivot points 1148, which can be hinged or just structured to bend in response to stress. Axial ribs 1444 further have one or more pivot points along the length of the rib. A distal end piece 1150 is attached to a distal end of axial ribs 1144 and to a corewire extension 1152. Proximal end piece 1146 is secured to the tubular suction extension while distal end piece 1150 is secured to corewire 1114 and allowed to slide over the suction extension. In embodiments where distal end piece 1150 is allowed to slide, pulling corewire 1114 proximally draws the end pieces closer to one another and forces the axial ribs to fold outward at the pivot points, thereby engaging the catheter wall. Conversely, pushing the corewire 1114 proximally causes the end pieces to separate and the axial ribs disengage the catheter. Manually actuated seal 1142 can further comprise a cover 1154 to cover the framework and provide the seal to restrict liquid flow past the engaged seal. Cover 1154 can comprise an elastomer, expanded polytetrafluoroethylene, nanofiber spun structure, or other fluid resistant covering that can be expanded in its sealed arrangement around the connecting section. In alternative embodiments where the distal end piece is fixed in position and the proximal end piece is attached to the corewire and allowed to slide over the suction extension, pushing the corewire 1114 distally pushes the end pieces closer to one another and forces the axial ribs to fold outward at the pivot points, thereby engaging the catheter wall. Conversely, in this alternative embodiment, pulling the corewire 1114 proximally causes the end pieces to separate and the axial ribs disengage the catheter.

FIGS. 19J-K illustrates an alternative embodiment of a connection section 1160 with a manually activated seal with a cage structure 1162. The cage structure 1162, which may have a structure, for example, of a stent or stent-like apparatus, functions similarly to the embodiment discussed above, although with actuation to an extended configuration achieved by pushing the corewire in a distal direction. A distal end 1164 of cage structure 1160 is fixed to the tubular structure of the connecting section. A proximal end of cage structure 1160, which is free to slide over the tubular structure, is attached to corewire 1114. Pushing or pulling corewire 1114 causes cage structure to extend upon distal sliding of corewire 1114, or contract in diameter upon proximal sliding of corewire 1114. Cover 1110 is located over cage structure 1160. Cage structure 1160 can offer more points of contact than foldable axial ribs 1144 with respect to cover 1110, which may provide for engagement with the catheter over a larger area of the interface.

FIGS. 20A-D depict a self-actuated engagement element for connection section 1170. In embodiments, self-actuated engagement element 1172 may comprise a self-actuating frame 1174 with a cover 1110 surrounding a tubular feature 1158 of the suction extension. To maintain the self-actuating frame 1174 in a compressed state, such as for delivery or withdrawal of the suction extension, self-actuating frame may be surrounded by sheath 1176. Sheath 1176 has a separate release control 1178 that is distinct from control wire 1114 such that sheath 1176 may be independently moved. Sliding sheath 1176 by pulling release control 1178 in a proximal direction releases self-actuating frame 1174, permitting self-actuating frame 1174 to extend and engage the guide catheter wall to place cover in a position to form a seal.

While it may be acceptable to fully remove sheath 1176 to the proximal fittings after self-actuating frame 1174 is released, sheath 1176 generally is not blocking flow and can be left in association with the connecting section. In some embodiments, advancing release control 1178 distal direction can cause self-actuating frame 1174 to compress within sheath 1176, allowing for easier removal of the suction extension. In embodiments, a distal portion of sheath 1176 may be angled outward and a proximal edge of self-actuating frame 1174 configured to engage sheath 1176 to facilitate compression of the self-actuating frame 1174 when sheath 1176 is advanced. Self-actuating frame generally is enclosed in a cover 1110 as described in detail above. In practice, such a combination has similarities in function to a balloon, while being self actuating, eliminating the need for fluid and a separate lumen.

Referring to a fixed connection section embodiment in FIG. 21, a sectional view is shown of a connecting section 400 of a suction extension within an engagement portion 402 of a guide catheter. The non-cylindrical nature of the cross section of connecting section 400 is readily visible. Due to the interface between the elements, the oval shape of connecting section 400 can be distorted relative to its shape separated from the guide catheter, especially if the undistorted length of the major axis of the connecting section 400 is greater than the inner diameter of engagement portion 402. Connecting section 400 can contact the inner surface of the lumen of engagement section 402 at two contact locations 404, 406. The size of contact locations 404, 406 generally depends on the dimensions of the elements, the shape of connecting section 400 and the material properties. It is generally not necessary to precisely define the boundaries of the contact locations.

As noted above, the non-cylindrical connecting section can be characterized with the major axis, minor axis and an average diameter obtained from the circumference. Based on these parameters, it is possible to specify significant aspects of the interface between connecting section 400 and engagement portion 402 with a difference between the major axis and the minor axis, with a difference between the major axis of an unconstrained connecting section 400 and the inner diameter of engagement section 402, and with the difference between the inner diameter of engagement section 402 and the average diameter of connecting section 400. For example, the difference between the major axis and the minor axis can be from about 30 microns to about 160 microns and in further embodiments from about 50 microns to about 140 microns. In some embodiments, the tolerance measured as a difference between the diameter of the inner surface of engagement section 402 and the average diameter of the connecting section can be, for example, no more than about 4 thou (1 thou= 1/1000 of an inch; 4 thou ˜102.6 microns), in further embodiments no more than about 3 thou (76.2 microns), in additional embodiments no more than about 1.75 thou (45 microns), in other embodiments from about 1 thou (25.4 microns) to about 1.75 thou (45 microns) and can be approximately zero within the measurement uncertainty. For embodiments in which the major axis of the connecting section separated from the guide catheter is larger than the guide catheter inner diameter, the difference between the major axis of unconstrained (i.e., separated from the guide catheter) connecting section 400 and the inner diameter of engagement section 402 can be from about 0 to about 250 microns, in further embodiments from about 15 microns to about 150 microns and in other embodiments from about microns to about 100 microns. A person of ordinary skill in the art will recognize that additional ranges of dimensions differences within the explicit ranges above are contemplated and are within the present disclosure.

Catheter components can be formed from one or more biocompatible materials, including, for example, metals, such as stainless steel or alloys, e.g., Nitinol®, or polymers such as polyether-amide block co-polymer (PEBAX®), nylon (polyamides), polyolefins, polytetrafluoroethylene, polyesters, polyurethanes, polycarbonates, polysiloxanes (silicones), polycarbonate urethanes (e.g., ChronoFlex AR®), mixtures thereof, combinations thereof, or other suitable biocompatible polymers. Radio-opacity can be achieved with the addition of metal markers, such as platinum-iridium alloy, tantalum, tungsten, gold, platinum-tungsten alloy or mixtures thereof, such as wire or bands, or through radio-pacifiers, such as barium sulfate, bismuth trioxide, bismuth subcarbonate, powdered tungsten, powdered tantalum or the like, added to the polymer resin. Medical grade PEBAX is available commercially loaded with barium sulfate, as well as with ranges of Shore hardness values. Generally, different sections of aspiration catheter can be formed from different materials from other sections, and sections of aspiration catheter can comprise a plurality of materials at different locations and/or at a particular location. In addition, selected sections of the catheter can be formed with materials to introduce desired stiffness/flexibility for the particular section of the catheter. Similarly, fitting components can be formed form a suitable material, such as one or more metals and/or one or more polymers.

In some embodiments, the guide catheter, suction extension or appropriate portions thereof comprises a thermoplastic polymer, such as the polymers listed above, with embedded metal elements, which reinforces the polymer. The wire can be braided, coiled or otherwise placed over a polymer tubing liner with some tension to keep the wire in place over the tubing liner. In some embodiments, a polymer jacket, such as a heat shrink polymer, can then be placed over the top and heated to shrink and fuse the cover over the structure, and/or the polymer tube can be softened with heat to allow incorporation of the metal reinforcements. Upon heating to a temperature over the softening temperature and/or heat shrink temperature of the polymer and subsequent cooling, the reinforcing metal becomes embedded within the polymer. In appropriate embodiments, a liner and a jacket can be the same or different materials. Suitable wire includes, for example, flat stainless steel wire or the like. Wire diameters can range from about 0.00025 inch (0.00635 mm) to about 0.004 inch (0.1 mm) and in further embodiments from about 0.0005 inch (0.013 mm) to about 0.003 inch (0.075 mm). For appropriate embodiments, braid picks per inch can be from about 20 to about 250 picks per inch and in further embodiments from about 50 to about 150 picks per inch. For appropriate embodiments, coils can be single or multiple filament coils having, for example, pitches from about 0.005 inch (0.13 mm) to about 0.1 inch (2.54 mm) and in further embodiments form about 0.01 inch (0.26 mm) to about 0.050 inch (1.27 mm). A person of ordinary skill in the art will recognize that additional ranges within the explicit ranges below are conceived and are within the present disclosure. The wire adds additional mechanical strength while maintaining appropriate amounts of flexibility. The wire can provide some radio-opacity although radiopaque bands generally would provide a darker and distinguishable image relative to the wire. However, the image of the wire can provide further visualization of the catheter during the procedure.

To decrease the chance of accidental removal of the radiopaque band from the catheter and to decrease the chance of the radiopaque band catching onto other objects within the vessel, a metal reinforcing wire can be used to cover or enclose the radiopaque band with the metal wire subsequently being embedded within the polymer. In some embodiments, a polymer jacket can be placed over the metal wire, which is correspondingly covering the radiopaque band(s), and the heat bonding embeds the radiopaque marked band also. If desired, placement of the marker band under metal wire can prevent the band from being separated from the catheter in the event that the wall is kinked or collapsed. If collapse or kinking of the catheter wall occurs, the braid-wire over the surface of the band collapses down over the marker band to prevent it from separating from the structure.

### Treatment Systems

The suction system described herein can be used effectively to remove blood clots from the vasculature, including the vasculature of the brain to treat acute stroke conditions. In particular, the narrow tip catheter of the '792 patent have performed well in human clinical trials to restore blood flow in persons with an acute embolic stroke with good patient outcomes. The device described herein may be expected to provide even better suction while maintaining access capability into vessels challenging to navigate. Nevertheless, for some acute stoke conditions or other embolic events, it can be desirable to use the suction catheter systems described herein with other medical tools for performing the therapy. Furthermore, specific desirable embodiments of proximal fittings are described in this section that provide for improved procedures for use of the suction extension described herein. In particular, adaptations of the proximal fittings provide for removal of a tubular extension of the suction extension from the guide catheter without passage through a hemostatic valve. In some embodiments, the proximal fittings can further comprise an additional branched fitting with a proximal end that can dock the proximal end of the suction extension to provide for convenient removal from the isolated locations behind a hemostatic valve to provide for convenient clearing of thrombus blockage of the suction extension and reinsertion. The thrombus blockage can be cleared through a flush delivered from a branch of docking Y-connector with the suction extension docked for quick replacement of the suction extension for the additional removal of further blockage form the blood vessel in the patient.

Also, the proximal fittings can be adapted with a pressure sensor that can provide valuable information about the status of the suction process. This pressure sensor can be in addition or an alternative to one or more pressure sensors in the wall of the catheter, as described above. A flow meter can be included in the proximal fittings or connected to high pressure tubing leading to a pump. The availability of the pressure information and/or flow information can be used to improve aspects of the procedure to increase efficacy and to reduce potential risks to the patient. In some embodiments, an electronically controlled valve can be connected to the tubing to the pump to provide for aspiration control without the need to power the pump on and off. The valve can have on/off setting or continuous adjustment of the degree of valve opening. A controller can be programmed to adjust the valve setting based on measurements of the pressure and/or flow meter.

Referring to FIG. 22, a treatment system 450 is shown comprising a guidewire 452, embolic protection system 454, suction catheter system 456, shown with guide catheter 458 and suction extension 460 separated, a percutaneous medical device 462, a microcatheter 464, a delivery catheter 466, proximal fittings 468, negative pressure device, e.g., pump or syringe, or the like, 470, and a display unit 472. Suitable components of proximal fittings 468 are described below. Not all embodiments of medical systems may have all of these components, and some medical system embodiments may have multiple components of each type, such as multiple distinct percutaneous medical devices. Suitable structures covering desirable embodiments for proximal fittings 468 are discussed in the following section.

Guidewires suitable for use in tortuous bodily vessels are described in published U.S. Pat. No. 10,518,066 to Pokorney et al., entitled “Medical Guidewires for Tortuous Vessels,” incorporated herein by reference. In some embodiments, embolic protection system 454 can comprise a guide structure to provide for delivery of the device, and for these systems a separate guidewire may or may not be used. Suction catheter systems 456 are described in detail herein, and the various embodiments described herein can be adapted for use with the medical systems as well as for use as stand-alone devices. If desired for particularly challenging device delivery, the medical system can include a delivery catheter 466, as described in the '938 application.

Embolic protection devices with small filter longitudinal extent and designed for suitable manipulations to facilitate delivery in vessels have been developed that are suitable for use in the medical systems described herein. See, for example, U.S. Pat. No. 7,879,062B2 to Galdonik et al., entitled “Fiber Based Embolic Protection Device,” and U.S. Pat. No. 8,092,483B2 to Galdonik et al., entitled “Steerable Device Having a Corewire Within a Tube and Combination with a Medical Device,” both of which are incorporated herein by reference. Additional fiber-based filter devices particularly designed for delivery into tortuous vessels are described in U.S. Pat. No. 8,814,892B2 to Galdonik et al. (hereinafter the '892 patent), entitled “Embolectomy Devices and Method of Treatment of Acute Ischemic Stroke Condition,” incorporated herein by reference. The '892 patent describes the use of the filter device as a clot engagement tool for use with an aspiration catheter. The '892 patent also envisions the use of supplementary structures to facilitate engagement of the clot. The DAISe™ clot removal system with a fiber-based filter is under development by MIVI Nueroscience, Inc. The use of supplementary structures are also contemplated in procedures described herein.

Microcatheters have been designed to allow for access to small blood vessels, such as cerebral blood vessels, and cerebral microcatheters are available commercially, e.g. Prowler Select™ (Cordis Neurovascular Inc.) and Spinnaker Elite™ (Boston Scientific Co.). Of course, the term microcatheter can cover a range of devices, and the present discussion can focus on catheters useful for the procedures described herein. In some embodiments, microcatheters can comprise a distal section that is narrower than a proximal section. However, in further embodiments, a microcatheter can have an approximately constant diameter along its length to facilitate delivery of other devices over the microcatheter. A narrow distal diameter allows for the catheter to navigate the tortuous vessels of the brain. The distal section can be highly flexible enough to navigate the vessels, but resilient enough to resist kinking. A microcatheter comprises at least one lumen. The microcatheter can then be used to deliver other treatment devices, aspiration, therapeutic agents, or other means of treating a condition. While microcatheters can have a selected size, in some embodiments, the microcatheters can have a distal outer diameter from about 1.0Fr to about 3.5Fr and in further embodiments from about 1.5Fr to about 3Fr, and a length from about 30 cm to about 200 cm and in further embodiments from about 45 cm to about 150 cm. A person of ordinary skill in the art will recognize that additional size ranges within the explicit ranges above are contemplated and are within the present disclosure.

With respect to percutaneous medical devices 762, suitable devices include, for example, clot engagement devices, angioplasty balloons, stent delivery devices, atherectomy devices, such as stent retrievers, and the like. Desirable thrombus engagement devices are described in U.S. Pat. No. 10,463,386 to Ogle et al., entitled “Thrombectomy Devices and Treatment of Acute Ischemic Stroke With Thrombus Engagement,” incorporated herein by reference. Stents may be, for example, balloon extendable, self-extendable or extendable using any other reasonable mechanism. Also, balloon extendable stents can be crimped to the balloon for delivery to engage a clot in a blood vessel. Some balloon-stent structures are described further, for example, in U.S. Pat. No. 6,106,530, entitled “Stent Delivery Device;” U.S. Pat. No. 6,364,894, entitled “Method of Making an Angioplasty Balloon Catheter;” and U.S. Pat. No. 6,156,005, entitled “Ballon [sic] Catheter For Stent Implantation,” each of which are incorporated herein by reference. Self-expanding stents are described further in U.S. Pat. No. 8,764,813 to Jantzen et al., entitled “Gradually Self-Expanding Stent” and U.S. Pat. No. 8,419,786 to Cottone, Jr. et al., entitled “Self-Expanding Stent,” both of which are incorporated herein by reference. Stent retrievers are described, for example, in U.S. Pat. No. 8,795,305 to Martin et al., entitled “Retrieval systems and methods of use thereof,” incorporated herein by reference.

Once the clot treatment process is completed, it has been found that it is advantageous to at least partially remove the tubular extension of the suction extension from the guide catheter before removing the guide catheter from the patient. If a portion of the tubular extension is removed through a hemostatic valve during this removal process, the isolation between the vasculature and the exterior of the patient can be lost since the proximal end of the tubular extension is not designed for closure. The loss of isolation between the exterior of the patient and the interior of the catheter system can result in an undesirable amount of bleeding as well as complicating the control of trapped thrombus associated with the nozzle In some embodiments, the fitting designs described here are intended to address these issues through the inclusion of a tubular storage area distal to a hemostatic valve and connected for access to the proximal end of the tubular extension. Several suitable designs are described herein. The loss of blood from this withdrawal of the tubular extension can be reduced or eliminated through the use of the docking branched manifold described herein. As noted in the discussion below, the fitting structures can be assembled for commercial elements or can be designed as a specific fitting particularly for the suction system and/or treatment systems described herein.

During procedures with the aspiration system, the tubular extension of the suction extension may be removed from the patient to clear a clot prior to reinsertion and further removal of thrombus. Clearing of the clot from the tubular extension generally involves removal from the guide catheter and out from a hemostatic valve. After the tubular extension is cleared of blockage, it is reinserted through the hemostatic valve back into the patient. The clearing of the clot generally involves the back flow of fluid from the proximal to distal ends. The fittings described herein allow for the docking of the connection section of the suction extension against a docking element in a docking Y-fitting for removal through the hemostatic valve. Once removed through the hemostatic valve, flush fluid can be delivered from one branch of the Y-fitting to flush the tubular extension without the need to provide further connections to the suction extension. The other branch of the Y generally comprises a hemostatic valve or the like through which the control structure passes, and the closed valve allows for the direction of the flush fluid through the suction extension.

The first fitting elements have been previously described in published U.S. patent application 2019/0183517 to Ogle, entitled “Suction Catheter Systems for Applying Effective Aspiration in Remote Vessels, Especially Cerebral Arteries,” incorporated herein by reference. The first fitting elements herein can be essentially the extent of the proximal fittings, but in the desirable embodiments herein, the proximal fittings further comprise a docking branched manifold. With the use of a docking branched manifold, the fittings can include further options for location of providing aspiration and/or delivery of perfusion liquids, ach as contrast dye or therapeutic compounds. Thus, while the earlier described proximal fittings can carry over to the first fitting element for engagement with a docking branched manifold, the first fitting elements can be designed, if desired, with less or different branching if certain functions are performed using the docking branched manifold. Thus, some of the embodiments described herein can be correspondingly simplified in some embodiments.

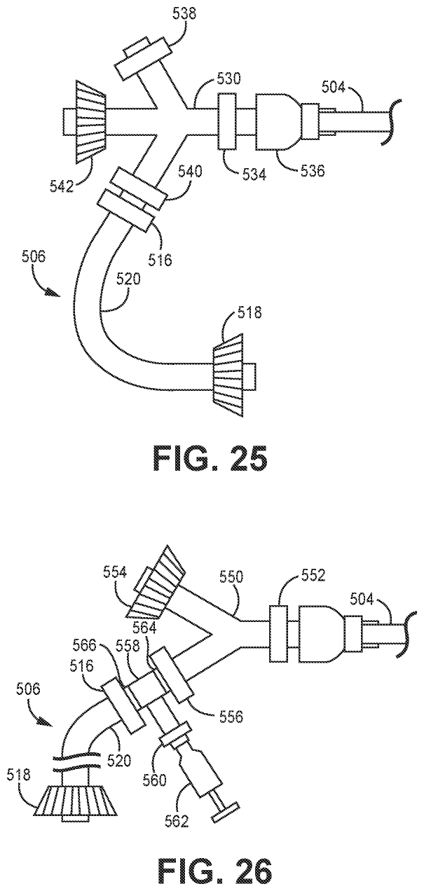

Three representative embodiments for the first fitting element of the proximal fittings providing withdrawal of the suction extension within hemostatic confinement are presented in FIGS. 23-26 in which the devices provide for holding a tubular extension of the suction extension within the manifold sealed behind a hemostatic valve or valves. As shown in FIGS. 23-26, the proximal fittings are assembled from a plurality of fitting components, and these fittings are designed to allow for aspiration from these first fitting elements. But if desired, one or more of the components can be manufactured as a unitary structure with the corresponding elimination of one or more sets of connectors, and particular configurations can involve various tradeoffs, such as convenience of use, cost, packaging, standards in the art, flexibility of design during use, or the like. As shown in FIGS. 23 and 24, the components are shown spaced apart, while for contrast, the multiple components are shown connected in FIGS. 25 and 26. Of course, for specific applications, additional components of the overall manifold can be assembled into the ultimate proximal fitting structure. For example, embodiments are shown below providing for attachment of a pressure sensor. Also, as shown below, additional components of the manifold can provide for docking and withdrawal of the suction extension in association with a fitting to provide for clearing of a blockage in the suction extension.

Referring to FIG. 23, fittings 500 comprises Y-branch manifold 502 suitable for connection with guide catheter 504, and extended hemostatic fitting 506. Guide catheter 504 can be any of the embodiments of guide catheters described above. Y-branch manifold 502 provides for multiple connectors with fluid communication with guide catheter 504. As shown in FIG. 23, Y-branch manifold 502 comprises three connectors 510, 512, 514, which can be Tuohy-Borst connectors, Luer connectors or other suitable connector. Connector 510 can be selected for connection with guide catheter 504. Connector 512 can be connected to a negative pressure source, such as a pump, or to further branched manifolds to provide for various connections such as for an infusion fluid source, generally with at least one connection to a negative pressure device. Connector 514 is configured to connect with extended hemostatic fitting 506. Extended hemostatic fitting 506 comprises connector 516 for a mated connection with Y-branch manifold 502, hemostatic valve 518 and tubular portion 520 between connector 516 and hemostatic valve fitting 518. Tubular portion 520 can have in some embodiments a suitable length for removing a tubular extension of a suction extension out from guide catheter 504 without passing any portion of the tubular extension or connecting section through the hemostatic valve, although a proximal control structure generally passes through the hemostatic valve, which is the possible configuration through the procedure. In this embodiment, it can be desirable for extended hemostatic fitting 506 to be sufficiently long that the distal end of a tubular extension to be proximal to a branch or branches, such as the branch leading to connector 512 to provide for aspiration or profusion from or into the guide catheter free from interference from the tubular extension. FIG. 24 depicts an alternative embodiment of first fitting element without a branch, which is suitable for use with a docking branched manifold configured to deliver aspiration. Unbranched first fitting element 522 comprises connector 524, unbranched tubular element 526 and hemostatic valve 528.

The length of tubular portion 520 can be selected according to the length of the tubular extension as well as potentially if desired a relevant length of Y-branch manifold 502, which collectively can be referred to as a tubular section for placement of the tubular extension with the connecting section in hemostatic isolation outside of the guide catheter. It may or may not be desirable to withdraw the tubular extension fully into tubular portion 520 such that the remaining portions of the manifold are open. In other words, it can be desirable for tubular portion itself to be at least as long as the tubular extension. With respect to unbranched tubular element 526 of FIG. 24, this element may or may not have a suitable length for the withdrawal of the tubular extension to be fully isolated within the unbranched tubular element 526. For the range of alternative embodiments considered for the first fitting elements of the proximal fittings of FIGS. 23 and 24, the dimensions of the tubular section can be appropriately identified in the particular structure. In general, tubular portion 520 of extended hemostatic fitting 506 can have a length from about 8 cm to about 55 cm, in further embodiments from about 9 cm to about 50 cm, and in other embodiments from about 10 cm to about 45 cm. A person of ordinary skill in the art will recognize that additional ranges of lengths within the explicit ranges above are contemplated and are within the present disclosure.

In alternative or additional embodiments, extended hemostatic fitting 506 can comprise a tubular element with two connectors on either end and a separate hemostatic valve with a Luer or other connector on the opposite end that connect to each other to effectively form an equivalent structure to that shown in FIG. 23. Similarly, one or more additional fitting components can be connected using suitable connectors between extended hemostatic fitting 506 and Y-branch manifold 502, such as additional branched elements, and similarly additional fitting components can be connected at connector 512 to provide additional features to the fittings, such as connection of a pressure sensor or other structures. Thus, while providing the ability to withdraw a tubular extension within the closed fittings, the proximal fittings can be adapted with suitable structure to provide desired functionality. While this discussion has focused on the assembly of multiple fitting components to provide an overall fitting structure, one or more of these components can be formed as integral parts of a corresponding unitary structure, such as the integration of Y-branch manifold 502 and extended hemostatic fitting 506 into a unitary structure through the replacement of connectors 514 and 516 with a unitary section of tubing, and similar integration can be performed for adding additional structure. The unitary structure incorporating the features of Y-branch manifold 502 and extended hemostatic fitting 506 comprises a branched manifold with an extended hemostatic valve portion, which can be a suitable alternative to the structure in FIG. 24. Thus, various combinations of connecting elements, redesigning unitary components, and the like can be implemented to form a desired proximal fittings design.

Referring to an alternative configuration of a first fitting element in FIG. 25, a three-branch manifold 530 is connected to guide catheter 504 and extended hemostatic fitting 520 is connected to a connector of one branch of three-branch manifold 530. Three-branch manifold 530 comprises first connector 534 connected to a proximal connector 536 of guide catheter 504, first branch connector 538, second branch connector 540 and hemostatic valve 542. Second branch connector 540 is connected to the extended hemostatic fitting 506, which is described in detail in the context of FIG. 24. First branch connector 536 can be connected to a negative pressure source directly or through a further branched manifold. Hemostatic valve 542 can be used for the introduction of supplemental treatment structures or other desirable devices. Again, the structure shown in FIG. can be further divided into additional components if desired. For example, the three branch manifold can be effectively formed using two sequential Y-branch connectors. Again, additional fitting components can be connected onto the proximal fitting structure in FIG. 25 to provide additional features as described above in the context of FIG. 24. Also similarly, one or more separate components of the proximal fittings can be constructed as a unitary structure. Thus, component accretion and/or combination/joining processes can be combined for designing of a desired proximal fittings configuration.

Referring to FIG. 26, a further embodiment of a first fitting element of the proximal fittings is shown with a symmetric Y-branch structure. As shown in FIG. 26, symmetric Y-branch manifold 550 comprises first connector 552 connected to guide catheter 504, branched hemostatic valve 554 and branched connector 556. Branched connector 556 is connected with T-branch fitting 558. T-branch fitting 558 has a T-connector 560 that is shown connected with negative pressure device 562, such as a syringe or a pump. T-branch connector 556 is further connected with extended hemostatic fitting 506, which is described in detail in the context of FIG. 24 including but not limited to the dimensions of the element. T-branch connector 556 comprises connectors 564, 566 for respective connection with mated connectors 556, 516. The structure shown in FIG. 26 can be formed with multiple components used to form the structure, such as a separate component with hemostatic valve 554 connected with a suitable connector to a mated connector on symmetric Y-branch manifold 550, which is correspondingly modified. Again, additional fitting components can be connected onto the proximal fitting structure in FIG. 26 to provide additional features as described above in the context of FIG. 24. Also similarly, one or more separate components of the proximal fittings can be constructed as a unitary structure. Thus, component accretion and/or combination/joining processes can be combined for designing of a desired proximal fittings configuration.

The proximal fittings including its various potential components can be formed from suitable materials for sterile assembly, which can involve in some embodiments subjecting the components to radiation. The components can be formed in either rigid and/or flexible materials such as polymers provided herein, and the connectors can be formed from suitable combination of materials for the formation of seals, such as elastomers. Rigid components can be formed, for example, from polycarbonate or other suitable polymer. The tubular portion 520 of extended hemostatic fitting 506 can be formed from a more flexible polymer, such as one or more of the polymers described above for the catheter body, for example, polyether-amide block co-polymer (PEBAX®), nylon (polyamides), polyolefins, polytetrafluoroethylene, polyesters, polyurethanes, polycarbonates, polysiloxanes (silicones), polycarbonate urethanes (e.g., ChronoFlex AR®), mixtures thereof, combinations thereof, or other suitable biocompatible polymers. As noted above, the various fitting structures can be assembled from additional components, added onto or subdividing the various components of the embodiments, and/or the components can be formed as integral structures correspondingly molded. Thus, particular designs can be assembled from existing commercially available components or all or a portion of the fittings can be produced specifically for these applications.

The proximal fittings can also be equipped with a pressure sensor to help guide the procedure. If a pump is used to supply negative pressure, the pressure set on the pump establishes a differential pressure limit. If fluid freely flows to the pump, the differential pressure in the conduits leading to the pump can be relatively low. If flow is effectively completely blocked, the gauge pressure in the line can be approximately the pump pressure, which is negative indicating suction. Intermediate pressure levels may be indicative of restrictions of flow due to normal catheter or suction extension configurations that can cause some flow resistance, or of less severe blockages to the flow from various potential sources. In any case, as explained further below, having a measure of the line pressure in the proximal fittings can provide valuable information to assist in the procedure.

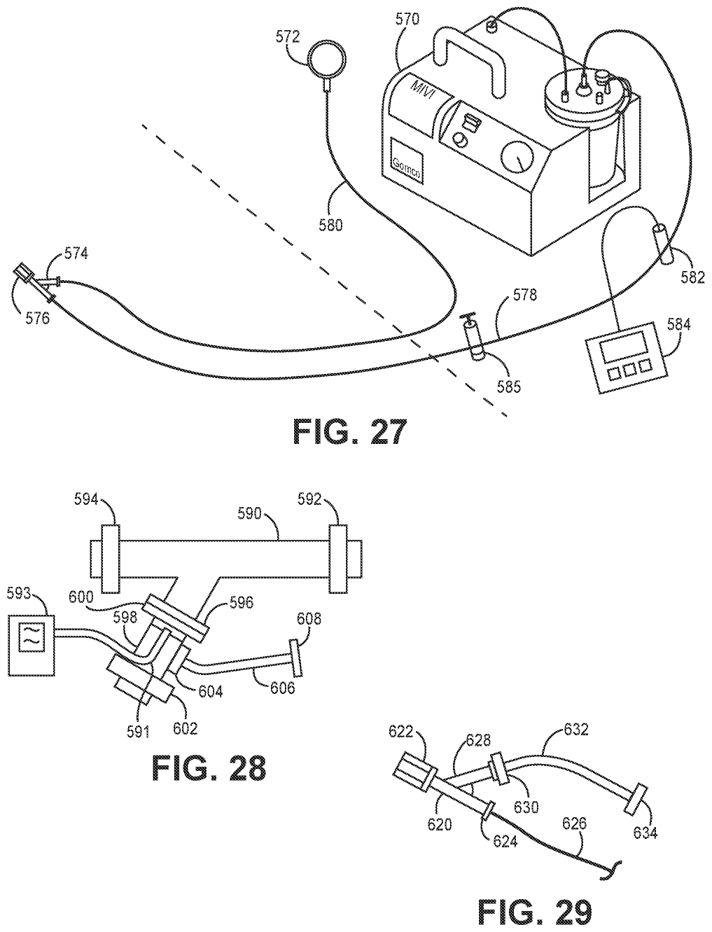

There are various possible configurations for a pressure sensor in association with the proximal fittings, and three representative embodiments are shown in FIGS. 27-29. Referring to FIG. 27, a pump 570 and pressure gauge 572 are connected to a Y-manifold 574 that comprises a connector 576 that can be attached to manifold connectors in the fittings connected to the guide catheter, such as shown in FIGS. 5 and 24-26. Pump 570 and pressure gauge 572 can be connected, respectively, using tubing 578, 580 to Y-manifold 574. The connections of tubing 578, 580 to Y-manifold 574 can be achieved at suitable connectors or they can be formed integral to the component. In this embodiment, pump 570 and optionally pressure gauge 572 may not be sterile, but no flow is intended to go to the patient from these devices. If the non-sterile components are appropriately isolated from the patient's fluids, the configuration can be acceptable even though the devices are not sterile. A selected length, for example 6 feet, dividing line to provide for appropriate sterile isolation is schematically denoted in FIG. 27 with a dashed line noted with an arrow.

Commercial aspiration pumps for medical applications, in which some specific pumps are noted above, can operate at gauge pressures from about −1 to about −26 inches of mercury (−25 mmHg to −660 mmHg). High-pressure tubing is also available for medical applications, e.g., from MIVI Neuroscience, Inc. (HFT 110™) or Penumbra, Inc. The high pressure tubing can have inner diameters from 0.07 inch to 1.0 inch, in further embodiments from about 0.075 inch to 0.5 inch and in other embodiments from 0.08 inch to 0.25 inch, and lengths of at least about four feet, in further embodiments at least about 6 feet, and in some embodiments from six feet to about 20 feet. A person of ordinary skill in the art will recognize that additional ranges of tubing dimensions within the explicit ranges above are contemplated and are within the present disclosure. The high-pressure tubing generally is reinforced to inhibit collapse of the tubing under negative pressure. The tubing is generally flexible and can be constructed, for example, from the types of polymers described herein for the construction of catheters.

Tubing 578 can be connected to a valve, such as an electronically controlled valve, 582. Valve 582 can control the delivery of aspiration to Y-manifold 574. While valve 582 can be a manually controlled valve, such as a pinch valve actuated with a lever, electronically controlled valves can provide easier and faster control of the valve. In particular, a solenoid valve can be a desirable design. While generally other electronic valves can be used as desired, commercially available solenoid valves can be attached to the exterior of the tubing or convenient use without contaminating either the flow or the valve. A commercial solenoid valve for mounting on tubing is available from Cole-Palmer® under the Masterflex series of two-way solenoid-pinch valves. A proportional solenoid pinch valve is available from IMI Norgren® under the Acro 900 series. As shown in FIG. 27, valve 582 is connected to controller 584. Controller 584 can be manually controlled and/or operated under programmed control. Controller 584 may receive input regarding measurements of other sensors and/or instructions from other control processors. Tubing 578 can additionally or alternatively have a manual valve 585, which can be operated with a pushing motion, twisting motion or other suitable manual engagement. In general, valves can be placed at any desired position along the tubing and in any convenient order.

A further embodiment of a fitting adapted with a pressure sensor is shown in FIG. 28. The fitting component in FIG. 28 comprises a Y-branch connector 590 with a distal connector 592, a proximal connector 594 and branch connector 596, and a pressure sensor component 598 with a first connector 600 shown connected with branch connector 590 and second connector 602. Pressure sensor component 598 further comprises pressure sensor 604 installed on the side wall of pressure sensor component 598. Electrical wires 606 extend from pressure sensor 604 and terminate at electrical connector 608, which can be a multi-pin clip or other suitable connector configuration. Electrical connector 608 can be suitable for connection to a suitable monitor or display. Commercial pressure sensor components for use as pressure sensor component 598 are commercially available, for example, from PendoTECH, Princeton, N.J., USA or MPS microfluidic pressure sensors are available from ELVEFLOW (Darwin Microfluidics), which also provides software for their operation. These components can be purchased sterile or they can be sterilized before use using conventional methods, such as using gamma irradiation. A pump or other negative pressure device can be connected to second connector 602 or other appropriate portion of the finally assembled proximal fittings, such as connectors associated with the docking branched manifold.

Another embodiment of a fitting component adapted with a pressure sensor is shown in FIG. 29. In this embodiment, Y-manifold 620 comprises a connector 622 for connection to other components of the proximal fittings and a connector 624 connected to tubing 626 for connection to a pump or the like. Y-manifold 620 further comprises a branch 628 adapted with a pressure sensor 630 at the end of the conduit. Pressure sensor 630 can be adapted on a connector cap or it can be bonded in a sealed configuration with branch 628, or otherwise adapted appropriately with a sealed attachment. Pressure sensor 630 is operably attached to electrical cable 632 which terminates at an electrical connector, such as a multi-pin clip. Pressure sensor dies or assemblies suitable for medical use are commercially available, such as from Merit Medical Systems, Inc. (Merit Sensors), which can be adapted for such connections.

It can be desirable to also integrate a flow meter in the proximal fittings to provide for measurement of flow in real time, and the flow measurement can be incorporated into a system control feature to coordinate the various operations of the system. Referring to FIG. 28, flow meter, shown as an optical fiber, 591 can be incorporated into the structure of Y-connector 590. Flow meter 591 can be based, for example, on an optical fiber Bragg grating in combination with an LED element, as described in Ruiz-Vargas et al., entitled “Optical Flow Sensor for Continuous Invasive Measurement of Blood Flow Velocity,” Journal of Biophotonics Vol. 12(10), October 2019, e201900139, incorporated herein by reference. An alternative example of a flow meter would be a Doppler ultrasound sensor placed near the distal end of the catheter on the proximal fittings or adjacent the proximal fittings with suitable sensors that can be adapted for this as described in Cannata et al., “Development of a Flexible Implantable Sensor for Postoperative Monitoring of Blood Flow,” Journal of Ultrasound in Medicine, November 2012, Vol. 31(11) pp 1795-1802, incorporated herein by reference.

Flow meter 591 can be electrically and/or optically connected to a display/controller 593 that can display flow readings from flow meter 591 and/or pressure readings, such as from pressure sensors 392, 394, 604. As shown, the optical fiber of flow meter 591 can exit Y-connector 590 in a sealed configuration for connection to controller 593. Controller 593 can provide the optical signal and analysis to allow for the pressure measurement, and controller 593 can comprise a display for the pressure measurement, or controller 593 can be interfaced with a separate display to show the corresponding pressure value. In embodiments, optical fiber 591 may interface with a wireless transceiver such that display/controller 593 may communicate wirelessly with optical fiber 591.

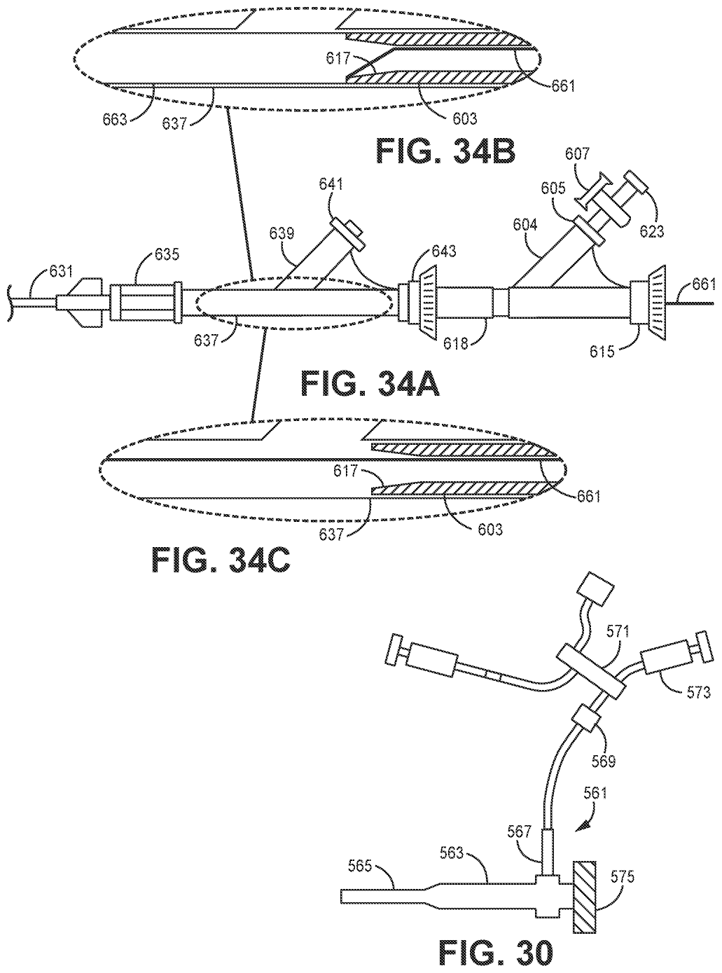

As described above, the proximal fittings can comprise a docking branched manifold to facilitate the process for de-clogging the tubular extension, and two specific embodiments are discussed further to elaborate on some potential features, although as with the first fittings element, a range of component designs can be suitable. A first representative embodiment of a docking branched manifold is shown in FIG. 30. As shown in FIG. 30, the docking branched manifold is shown with a first fluid source, a second fluid source and an aspiration source. In alternative embodiments, only a first fluid source can be used, or only a first fluid sour and an aspiration source can be used. Similarly, only a first fluid source and a second fluid source can be used. In further embodiments, a third or more fluid sources can be introduced. Docking branched manifold 561 comprises tubular body 563, docking inlet tube 565, side port and channel 567, and a proximal hemostatic valve 569 along tubular body 563 proximal to side port and channel 567. Side port and channel 567 connects with valve 571, access manifold 573, first fluid source 575, second fluid source 577, and aspiration source 579. Fluid sources 575 and 577 can comprise a reservoir, a delivery system, such as a syringe, a pump, or the like, and may optionally include a valve. Suitable valves can include, for example, a stopcock, a flow control switch such as available from Merit Medical, various mechanical or powered valves, or the like. Aspiration source can comprise a pump or other negative pressure device along with appropriate pressure tubing, and can optionally further be associated with a separate valve.

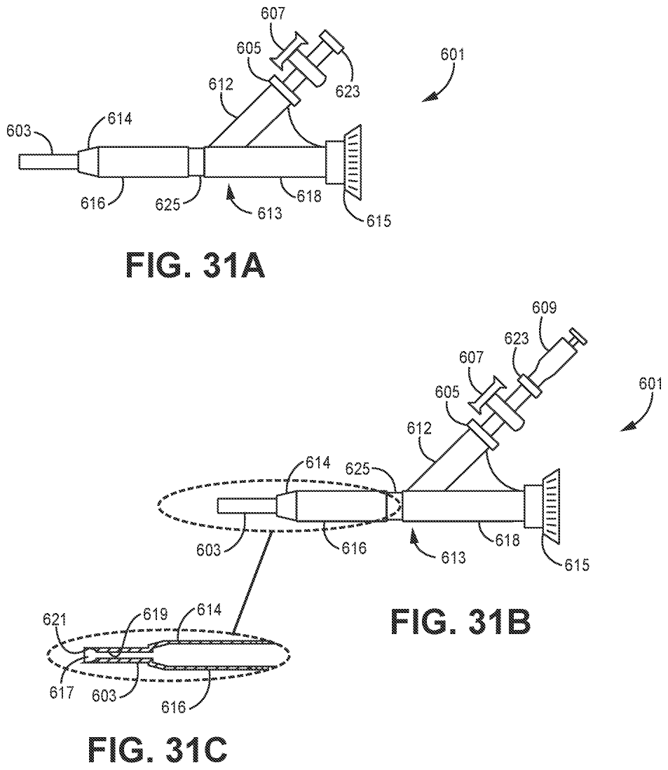

A second representative embodiment of a docking branched manifold is depicted in FIGS. 31-34 of a docking branched manifold 601 that may be used to remove a suction extension, clear any thrombus or other matter associated with the suction extension, and return the suction extension into the patient for the collection of additional thrombus. FIG. 31A illustrates a side view of docking branched manifold 601. The docking branched manifold 601 comprises an input tubular segment 603 at a distal end. Proximal to input tubular segment 603 is a first branch 612 with connector 605. In embodiments, source valve 607 is connected to docking branch manifold 601 at connector 605. Source valve 607 has a second port 623. Source valve 607 may be a 2-way valve or it may be a multiple port valve. In some embodiments, source valve 607 is a stopcock, although other flow control elements can be used and may be desirable, such as some valves described above. Source valve 607 may be in fluid communication with a fluid source and configured such that opening source valve 607 permits fluid to flow into docking branched manifold 601 and closing source valve 607 blocks fluid from flowing into docking branched manifold 601. An example of a fluid source such as a positive pressure device, such as a pump or pressurized container, loaded syringe 609 or the like is depicted in FIG. 31B. A branch of docking branched manifold 601 generally comprises a hemostatic valve 611 to allow for passage of a control structure associated with the suction extension.

Docking branched manifold 201 generally comprises a tubular body 613 which can comprise a tapered connector 614 connecting with input tubular segment 603, although the precise configuration of the connecting section is generally not significant. In some embodiments, tubular body 613 of docking branched manifold 601 can comprise a distal section 616 comprised of a material selected for sealing within a hemostatic valve and a proximal section 618 comprising a different material from the distal section that may be molded to further comprise the Y-branch. A connector 625 can be used optionally to join distal section 616 and proximal section 618, and connector 625 can be made of a suitable material. Connector 625 may or may not be visible from the exterior and may or may not alter the external diameter, the internal diameter, or both diameters. If a suitable material is selected, tubular body 613 can be formed from a single material.

FIG. 31C depicts a fragmentary sectional view of docking branched manifold 601 showing a distal portion of input tubular segment 603 comprises a docking structure 617. Docking structure 617 may be configured to releasably retain a proximal end of a suction extension, such as any of the embodiments described above. For example, docking structure 617 may use an interference fit to secure the proximal end of a connection section of a suction extension. In embodiments, docking structure 617 can be configured with an internal tapering of the internal walls 619 of input tubular segment 603. For example, an interior surface 621 of input tubular segment 603 may taper inwards until an interior diameter of tubular input is less than an outer diameter of the distal end of the suction extension. In additional or alternative embodiments, docking structure 617 may have a flange on interior surface 621 of input tubular segment 603, which can be considered to be an infinitely sharp taper. In embodiments, docking structure 617 may also comprise a structure on an interior surface 621 of tubular input segment 603 configured to interface with a corresponding structure at the proximal end of the connection section of the suction extension. For example, docking structure 617 may include a detent on an interior surface 621 of tubular input segment 603 configured to interface with an indent on an exterior surface of tubular extension. In general though, the docking structure can be any suitable structure, such as a narrowing tubular structure, that provides for an, at least, approximately fluid tight fit of the proximal end of the connection section of the suction extension.

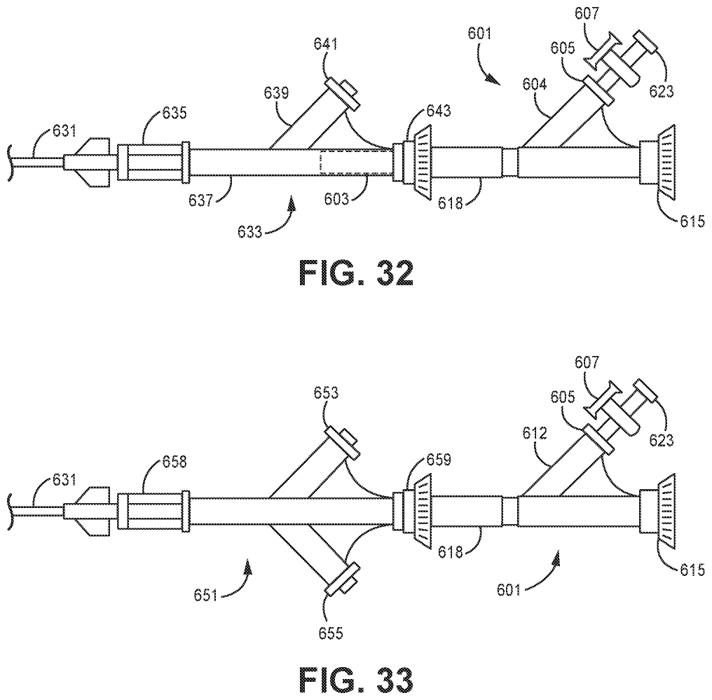

As illustrated in the fragmentary view of FIG. 32, a suction catheter system generally comprises a guide catheter 631 and a Y-branch manifold 633 is shown as the first fitting element, and any of the guide catheter embodiments and first fitting elements above can generally be used for this configuration. As depicted in FIG. 32, the docking branched manifold is the structure shown in FIG. 31A, and the alternative structures described in the context of this Fig. apply equally to the embodiment of FIG. 32. First fitting element 633 comprises a connector 635, tubular body 637, branch conduit 639 with a connector 641, and hemostatic valve 643. Similarly, other embodiments of the first fitting elements and docking branched fittings can be adapted into the assembled system. Docking branch manifold 601 can be designed to interface with Y-branch manifold 633 with proximal section 618 inserted through hemostatic valve 643. It should be understood that various manifold configurations are within the scope of this application. For example, the disclosed suction catheter system is not limited to the two pathways available in Y-branch manifold 633. For example, an unbranched first fitting element, as shown in FIG. 24B can be used. For another example, FIG. 33 depicts a suction catheter system including tri-branch manifold 651. Manifolds with additional branches may be used as well. Alternatively, manifolds may be connected to one another creating additional pathways. For example, an additional manifold could be attached at connector 653 or a second connector 655. Tri-branch manifold 651 connects with guide catheter 631 at connector 658. At a proximal end of tri-branch manifold 651, input tubular section 603 is inserted through hemostatic valve 659 to provide for docking with the suction extension within Y-branch manifold 633. Similarly, first fitting elements can comprise an integral structure or a structural component serving as an extended hemostatic fitting, such as shown about in FIGS. 24-26, that provides for removal of the tubular extension of the suction extension out from the guide catheter within a hemostatic environment, and the structures in FIGS. 30-34 can be corresponding interpreted to include this capability based on an adjustment of structure of dimensions.

FIG. 34A shows the assembled system of FIG. 32 with a suction extension deployed through the components, and control wire 661 is shown extending from hemostatic valve 615. FIG. 34B shows a sectional view of a portion of the suction catheter system depicted in FIG. 34A. Control wire 661 passes through docking branch manifold 601 and is secured to suction extension 663. As noted above, in embodiments in which the docking branched manifold is configured with a connection to a negative pressure device, a first fitting element with a branched manifold can be replaced with a first fitting element that is not branched, if desired, although the system can optionally provide aspiration from a selected connector from a plurality of available connectors or the connection of a manifold of the first fitting element can be used to deliver contrast dye or a therapeutic compound as an alternative to connection to a negative pressure device.

FIG. 34B illustrates suction extension 663 docked in docking structure 617. However, control wire 661 can be manipulated, for example by pushing on it thereby exerting an axial force in the distal direction, to release suction extension 663 from docking structure 617 and reintroduce suction extension 663 into the patient. Conversely, when suction extension 663 is not docked, control wire 661 can be manipulated to draw a proximal end of suction extension 663 into docking structure 617 until the tubular extension of suction extension 663 is secured. For example, control wire 661 could be pulled in the proximal direction until suction extension 663 forms an interference fit with a tapered portion of input tubular section 603. Alternatively, as shown in FIG. 34C, control wire 661 could be extended such that control wire extends entirely through docking structure 617 and thereby the tubular extension of suction extension 663 is distal to docking branched manifold 601.

Once suction extension 663 is docked in docking structure 617, docking branch manifold 601 can be separated from Y-branch manifold 633 such that suction extension 663 is withdrawn proximally through hemostatic valve 635. With the structures separate, source valve 607 may be opened to allow fluid to flow into docking branch manifold 601, through docking structure 617, and subsequently through suction extension 663. The flow of fluid can dislodge a thrombus or other matter trapped within the tubular extension of suction extension 663. Examples of fluids include, for example, sterile water, saline solutions, contrast dye, or other sterile fluids. If the procedure is ongoing, once suction extension 663 is clear of blockage, it may be reinserted through hemostatic valve 635 and into Y-branch manifold 633. Once docking branch manifold 601 is reinserted and secured within Y-branch manifold 633, control wire 661 may be used to disengage suction extension 663 from docking structure 617 and reintroduce the tubular extension of suction extension 663 into the patient for the collection of additional clotting material from the occluded blood vessel.

The particular embodiment of the docking branched manifold in FIGS. 31 to 34 are a representative embodiment, but other embodiments can have more than two branches with appropriate additional connectors, additional flow control elements, different angles for the branches, and the like. In particular, features described in the context of FIG. 30 can be adapted for the second representative structure in FIGS. 31-34. For example, the first branch of the docking branched manifold can comprise a source valve that controls the flow that can originate from a fluid source or flow to an aspiration source, such as a pump, to aspirate fluid from the manifold. Rather than using further branches off of a first branch, additional branches can be provided on the manifold to provide for access to additional fluid sources and/or an aspiration source, which would be similar to the additional branches for the first branched manifold of the proximal fittings shown in FIG. 25. A person of ordinary skill in the art can adjust the design based on the functional constraints based on the teachings herein.

The docking branched manifolds generally have suitable dimensions for convenient handling and manipulation and the interior dimensions are suitable for the handling of the various devices described herein. The components of the docking branched manifold can be formed in either rigid and/or flexible materials such as polymers provided herein, and the connectors can be formed from suitable combination of materials as long as they are suitable for the intended function of the component. Rigid components can be formed, for example, from polycarbonate, polyimides, metal or other suitable polymers. The portion of the docking branched manifold that gets secured in the hemostatic valve of the proximal fittings should have sufficient mechanical strength to avoid getting crushed by the hemostatic valve, which can be accomplished through the appropriate selection of material and wall thickness. In embodiments, tubular portions can be formed from a more flexible polymer, such as one or more of the polymers described above for the catheter body, for example, polyether-amide block co-polymer (PEBAX®), nylon (polyamides), polyolefins, polytetrafluoroethylene, polyesters, polyurethanes, polycarbonates, polysiloxanes (silicones), polycarbonate urethanes (e.g., ChronoFlex AR®), mixtures thereof, combinations thereof, or other suitable biocompatible polymers. As noted above, the various fitting structures can be assembled from additional components, added onto or subdividing the various components of the embodiments, and/or the components can be formed as integral structures correspondingly molded. Thus, particular designs can be assembled from existing commercially available components or all or a portion of the fittings can be produced specifically for these applications. In embodiments, portions of the components may be translucent or transparent. It may be beneficial for a user to be able to visually inspect the internals of the components. In some procedures it may be desirable for the user to visually determine when the suction extension is within a manifold or engaged with a docking structure. Thus, transparency in particular is a consideration for the fittings at the location where the docking structure would be located so that visual examination can help to confirm docking along with physical tactile evaluation. In some procedures, it may be desirable for the user to visually inspect tubular extension for a trapped thrombus or other debris prior to removing the tubular extension from the hemostatic environment.

The use of the aspiration systems described herein involves the manipulation of a control structure, such as a control wire, to move the body of a suction extension within a guide catheter. The movement generally involves extending the tubular extension from the distal end of the guide catheter as well as removing the suction extension from the proximal end of the guide catheter. In some embodiments, the guide catheter does not include a stop or other interfacing structure to engage the connection section of the suction extension to prevent the movement of the connection section of the suction extension from the distal opening of the guide catheter. If the connection section of the suction extension passes through the distal opening of the guide catheter, it may be difficult to recover the procedural objectives without removal of the guide catheter from the patient, which can result in undesirable delays that provide risk to the patient and add to costs associated with procedure times. While markings can be provided on a control structure to instruct the health care professional not to ever insert the control structure, such a system may involve an undesirable level of risk with respect to user error.

A handle can be secured to the control structure at or near the proximal end of the control structure to facilitate gripping the control structure as well as to prevent over insertion of the control structure into the guide catheter. The grip or handle then can have a shape or sufficient thickness orthogonal to the control structure to inhibit insertion of the handle through a hemostatic valve. Various configurations can be suitable for a grip or handle, although generally they should be easily gripped by a heath care professional with one hand for manipulation during a procedure. A handle can be fixedly attached to the control structure, or the grip can be repositionable on the control structure. If the grip is repositionable, the proximal end of the control structure can be bent, tied, twisted, or otherwise altered to make it difficult or impossible to remove the grip without destroying a component. For use, the handle should be appropriately anchored if it is not permanently secured at a particular position. If the handle can be repositioned, for example to allow for use with different fittings or guide catheter embodiments, the securing of the handle can be provided with a screw, a clip, snap, other fastener, or other appropriate structure, which can be engage during manufacture of a product or by the user with appropriate instruction.

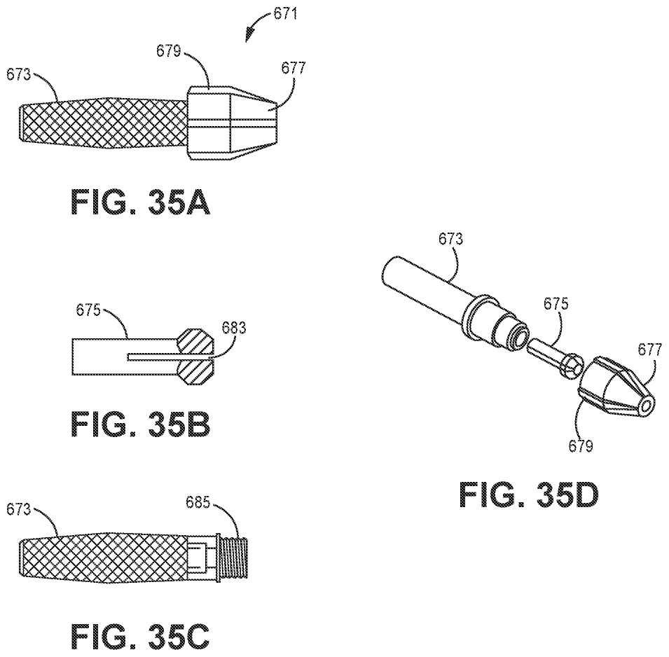

In one representative embodiment, a handle is provided by a pin vise. FIGS. 35A-35C illustrate an embodiment of a pin vise 671 with a knurled collet holder 673, a collet 675, and a head 677. In embodiments, head 677 may have one or more ribs 679. Ribs 679 may make it easier to turn head 677 in order to either retain or release control wire 681. Further, ribs 679 may help prevent pin vise 671 from rolling, such as when placed on a surgical tray or table. Collet 675 has a thru hole 683 configured to receive control wire. When control wire is inserted into thru hole 683, rotating head 677 about threads 685 in a first direction causes collet 675 to clamp down on control wire in a vise like grip, and rotating head 677 in an opposite direction causes collet 675 to release control wire. When control wire is secured by collet 675, collet holder 673 may be manipulated to exert control over a control wire and a corresponding suction extension. For example, twisting collet holder 673 may place torque on a control wire. Pulling collet holder 673 axially may withdraw a suction extension from a patient and/or cause suction extension to dock within a docking structure. Similarly, pushing collet holder 673 axially may release a suction extension from a docking structure and/or reposition a suction extension within the vasculature of a patient.

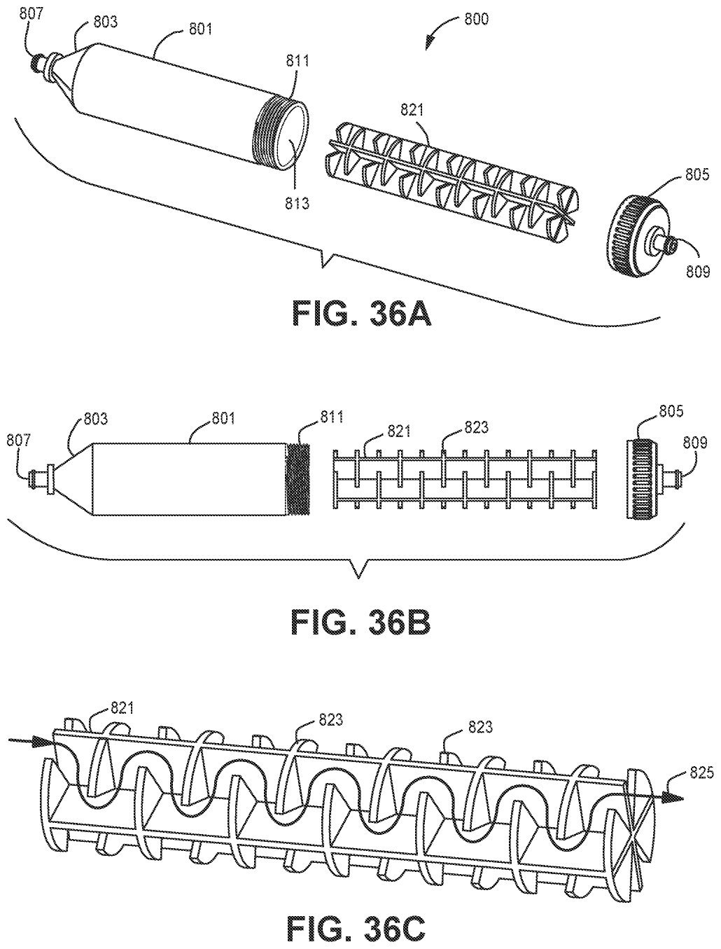

Aspiration systems as described herein may include a filter with an aspiration source and adjacent to proximal fittings for manipulating the aspiration catheter. Examples of filters are illustrated in FIGS. 36A-36C, 37A-37B, 38A-38B, and 39A-39D. Components of the filters can be constructed from metal and polymers described above of the fittings components. Filter material can be constructed from materials as described below. The filter designed to remove clots from the flow from the fittings is attached upstream from the high pressure tubing, such as immediately upstream and connected to the high pressure tubing. The high pressure tubing is generally at least six feet long to separate sterile and non-sterile components. The other connector of the filter can be connected directly or indirectly to the remaining fittings, and various configurations of the fittings and relative positions are described herein.

Referring to FIGS. 36A-36C, filter 800 has a tubular body 801, a forward portion 803, and an end cap 805. In embodiments, forward portion 803 is gradually tapered. In embodiments, forward portion 803 is conical. Forward portion 803 generally comprises connector 807, such as a male luer connector. Removable end cap 805 generally comprises connector 809, such as a female luer connector. Connections 807, 809 and tubular body 801 are in fluid communication such that fluid may pass through filter 800. At least one of connections 807, 809 is readily attached to high pressure tubing of the aspiration system, while the other end is attachable to proximal fittings. In embodiments, connections 807, 809 are luer connectors. Tubular body 801 has a larger diameter than the relatively small diameter high pressure tubing of the aspiration system. Accordingly, debris, such as clots, which may impede flow within the high pressure tubing of the aspiration system may collect within tubular body 801 without impeding flow rates within the high pressure tubing. End cap 805 can be bonded to tubular body 801, such as with adhesive or heat bonding or, in further embodiments, can releasably engage tubular body 801 with a friction fit, a screw connection, a bayonet engagement or other convenient engagement.

In some embodiments, the average diameter of tubular body 801 can range from about 0.4 inch to about 5 inch, in further embodiments from about 0.5 inch to about 3.5 inch, and in further embodiments from about 0.6 inch to about 3 inch. While the diameter along tubular body 801 can conveniently be about constant, this diameter can reasonably vary without altering function within the average specifications. The length of tubular body 801 can be from about 0.5 inch to about 8 inches, in further embodiments from about 0.75 inch to about 7 inches, and in other embodiments from about 1 inch to about 6 inches. A person of ordinary skill in the art will recognize that additional ranges within the explicit ranges above are contemplated and are within the present disclosure. In general, filter 800 can be formed from suitable polymers, such as polycarbonate, acrylic polymers, polyamides, high-density polyethylene, polyesters, copolymers thereof and the like. Luer fittings can comprise multiple components and can be appropriately constructed or obtained commercially from suppliers, such as Merit Medical.

Tubular body 801 may contain additional structure, such as filters matrices or other material, designed to catch clots while having little impact on flow rate through filter 800 and attached tubing. FIGS. 36A-36C illustrate an exploded view of filter 800 with corrugated filter element 821. Tubular body 801 has a threaded portion 811 that interfaces with end cap 805. Corrugated filter 821 is configured to fit within an interior chamber 813 of tubular body 801 and is fully contained therein when end cap 805, with mated threads within the cap, is secured to threaded portion 811. Corrugated filter 823 has a plurality of ribs 823 arranged in a pattern to create a set number of flow paths through the filter 821 when working in concert with tubular body 801. Differently configured corrugated filters 821 may have a different number of flow paths 825 as well as optionally different configurations of ribs. When a clot enters corrugated filter 821, it can proceed down one of the flow paths 825 and becomes lodged against one of ribs 823. Fluid may continue to flow around the ribs 823 even when a clot is lodged, such that the flow rate within the attached high-pressure tubing is substantially unaffected by the clot lodged within corrugated filter 821.

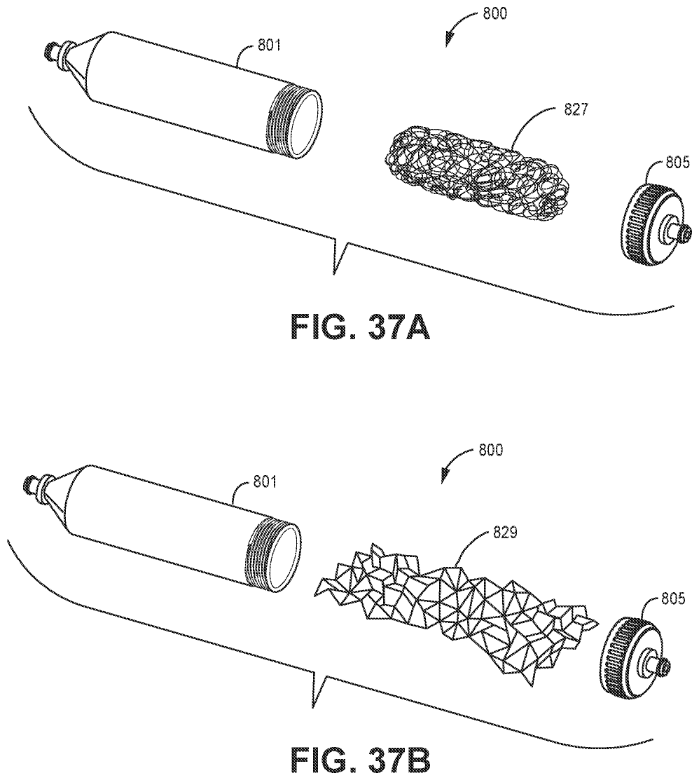

FIGS. 37A and 37B illustrate filter structures having different filter elements. A fiber matrix filter element 827 has a fiber matrix that can trap clots within the fiber elements while allowing fluid to pass through generally unimpeded. The fiber matrix filter element 827 can comprise, for example, cellulose fibers, polyester fibers or other reasonable fiber elements. Folded matrix element 829 is a folded material that can trap clots within the material folds while fluid flows around the folds and/or through the folded material. Folded matrix element 829 can comprise folded up filter paper with appropriate pore size to allow passage of blood components through the filter paper. Filter elements 821, 827 or 829 generally are sterilized for use due to their proximity to the proximal fittings, and an appropriate sterilent can be selected such as vapor or radiation sterilization. Generally, these would be shipped in sterile condition, and the sterile packaging opened for assembly under appropriate sterile conditions in a procedure room.

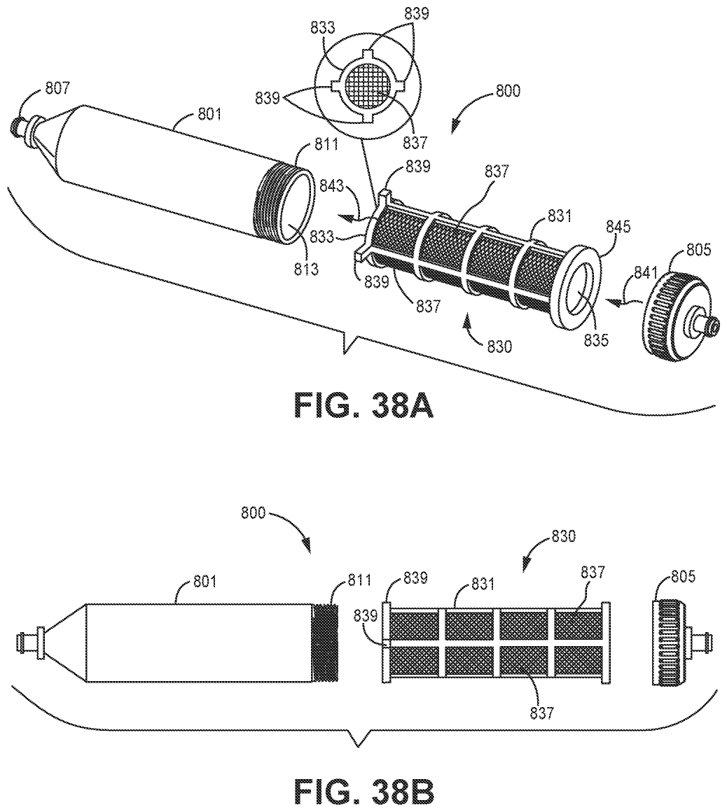

Referring to FIGS. 38A-B, an embodiment of filter 800 is shown in disassembled form with a screen based filter element. Filter element 830 comprises open end 835 formed through engagement ring 845 that engages end cap 805 in an effectively seal configuration when end cap 805 is affixed to tubular body 801. Filter element 830 further comprises an optional frame 831, closed end ring 833, filter screen 837, and struts 839. If filter screen is sufficiently self-supporting, such as a woven or welded metal screen, frame 831 may not be used, and rings 833 and 845 can be directly attached to the filter screen. Struts 839 stabilize filter element 830 within tubular body 081 in the assembled filter, while providing for flow past the ring 833. The interior of closed end ring 833 can have filter screen 837 (as shown in the balloon insert of FIG. 38A) or closed completely. Filter 800 of this embodiment can be designed to provide for insertion of filter element 830 into tubular body 801 in either direction. In principle, filter 800 in the embodiments of FIGS. 38A-B can be operated with flow in either direction, and in some embodiments of this filter, it is desirable to have flow enter into opening 835 into the interior of filter element 850 with flow with clots restrained passing through filter screen 837 to exit filter 800.

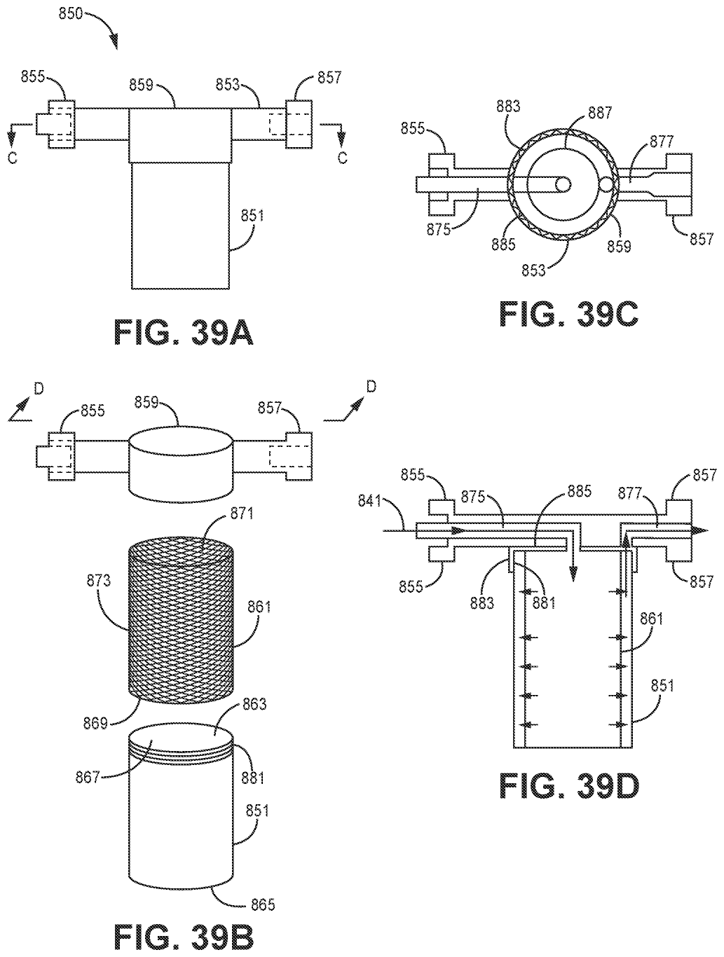

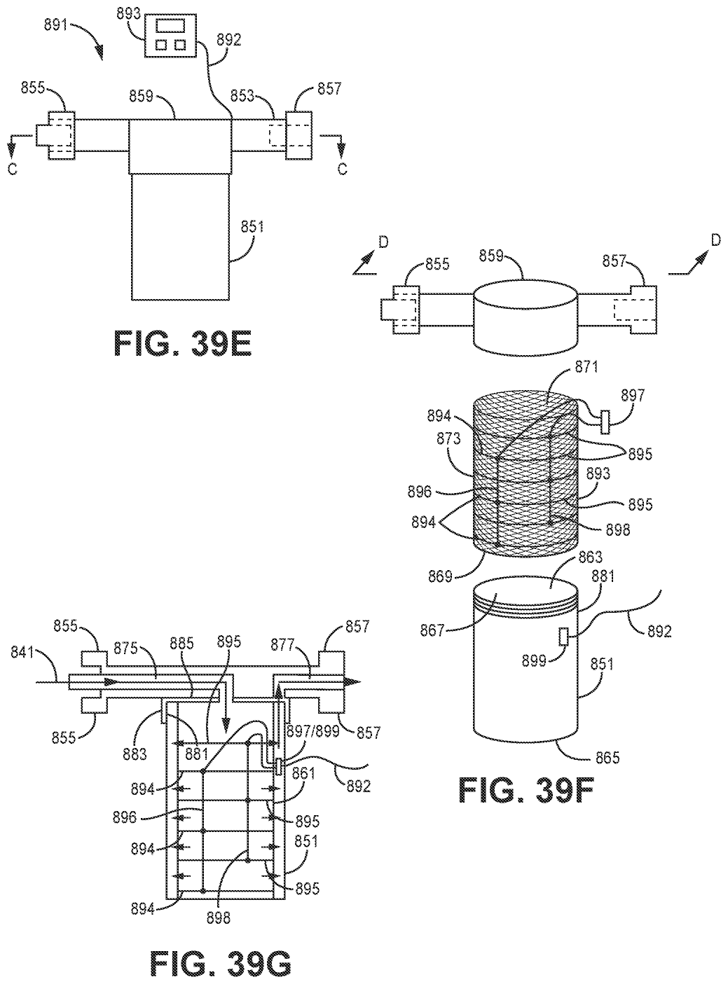

Referring to FIGS. 39A-39D, filter 850 has an alternative configuration to the filters in FIGS. 36-38, in which the configuration of FIGS. 39A-D provide for easier access into the interior of the filter since connections to the filter are not connected to the filter body. Filter 850 has filter body 851 and end cap 853. End cap 853 includes connections 855, 857 configured to attach with high-pressure tubing and proximal fittings. In embodiments, connections 855, 857 are luer fittings. Filter body 851 interfaces with a central portion 859 of end cap 853. In embodiments, filter body 851 and central portion 859 of end cap 853 have corresponding threads 881, 883, respectively, such that filter body 851 may screw into central portion 859 and form a seal. If desired, central portion 859 can comprise a washer or gasket 885 to engage with screwed on filter body 851. Filter body 851 further has an open top end 863 opposite closed bottom end 865, and an interior chamber portion 867 there between. End cap 853 has a first channel 875 extending from a first connection 855 to about the center of central portion 859, such that the channel is in fluid communication with the interior chamber 867 of filter body 851. First channel 875 then bends, e.g. about 90 degrees, towards filter body 851 to direct the flow. End cap 853 has a second channel 877 extending from a second connection 857 to about the perimeter of central portion 859, where second channel 877 bends, e.g. about 90 degrees, towards the edge of filter body 851 outside of area constrained by washer/gasket 883, such that flow outside of screen filter element 861 can flow to second channel 877.

Filter 850 may have a filter element 861 or similar filter structure. Screen filter element 861 is configured to fit within interior chamber portion 867 of filter body 851 and is fully contained therein when end cap 853 is secured to filter body 851. Filter element 861 optionally has a closed end 869 at bottom end opposite open top end 871 and mesh screen 873 there between. In some embodiments, closed end 869 engages the bottom of filter body 851 to restrict clots from exiting screen filter element 861. Closed end 869 can alternatively have a screen to allow flow through the end. Fluid entering, for example, through the open end 871 of filter element 861 passes through the screens 873 in order to exit filter 850. Filter element 861 should be sized to leave an appropriate gap between the filter element 861 and the wall of chamber portion 851 as well as to leave a flow path to the exit of interior chamber portion 851. For example, as shown in FIG. 39D, filter element 861 may have an outer diameter that is less than an inner diameter of chamber portion 851. Flow 841 enters filter element 861 through first channel 875. In embodiments, filter element 861 has a height that roughly matches a height of filter body 851, such that filter element 861 is held in position when filter body 851 is secured to end cap 853. In some embodiments, top end 853 can comprise a washer or the like to engage the top of filter element 861 when filter body 851 is engaged with end cap 853.

In embodiments, central portion 859 of end cap 853 may have a lip, protrusion and/or gasket that engages the top of filter element 861. A gap should be maintained between the wall of chamber portion 851 and filter element 861 such that flow 841, upon passing through screen 837 may continue between filter element 861 and wall of chamber portion 851, ultimately exiting in line filter 850 through connection 857. It should be recognized that flow 841 can be reversible and filter 850 may work with flow entering connection 857 and exiting through connection 855, but collection of clots is not necessarily equivalent for the two flow directions. A person of ordinary skill in the art can adjust these designs to have other functionally equivalent configurations based on this teaching. For example, the inclusion of O-rings, washers, gaskets, or the like may be used for seals to direct flow 841 and are not beyond the scope of this disclosure. In addition, while FIG. 39C depicts first channel 875 and second channel 877 connecting to inlets and outlets in a linear configuration, there is no functional need for this configuration, and the respective inlets and outlets can be placed at a selected angle relative to each other around the circumference as long as the inlet and outlet do not interfere with each other. The depicted linear configuration can be convenient for a range of setups.

Mesh screens 837 may be sized appropriately to capture clots while letting fluid flow essentially unimpeded. Since the purpose of the mesh screens is to remove clots that can impede flow through the tubing and not to purify blood for the patient, the pore size through the screen need not be particularly small. Pore sizes less than 1 millimeter and in further embodiments less than 0.5 millimeter may be adequate, and generally the pore sizes should not be too small, such as greater than at least about 0.1 mm. Similar effective filter sizes can be considered for the other embodiments. For meshes with relatively large pores, fibers can be included in the filter to help trap the clots, and gravity can further assist with the trapping clots, especially with a configuration, such as shown in FIG. 39. The packing of fibers can be selected to facilitate clot capture without significantly constricting flow or excessively obscuring visualization inside the filter. In embodiments, filter body 851 may be transparent, allowing for a visual assessment of debris trapped within filter 850. The ability to identify whether or not a clot has been captured within the filter, if it is transparent, can improve safety and help to guide the practitioner in performing the procedure.

An instrumented embodiment of the filter is shown in FIG. 39E-G. Referring to FIG. 39E, filter 891 comprises an electrical connection 892 connecting filter 891 with controller 893. Controllers for making electrical measurements on biological fluids are available commercially, such as electrical chips from Analog Devices, Inc., such as ADuCM355 chip. Generally, the controller provides a low voltage ac current, but a direct current could be applied. Electrical connection 892 can connect with electrodes within the filter housing in various ways. Generally, electrodes for the measurement can be located on the filter material to provide a relatively direct measurements influenced by clot captured in the filter material. The wires connected to the electrodes pass through the filter container, which can be through cap 859 or filter body 851. If the filter material is not exchanged, the electrodes can be hard wired, or alternatively an optional connecting clip can be used to disconnect the electrodes to allow for replacement of the filter material within the filter cartridge.

FIG. 39F is analogous to FIG. 39B with a filter element 893 having integral electrodes. In the depicted embodiment, filter element has electrodes encircling filter element 893 with three electrodes 894 of a first polarity/phase and three electrodes 895 of a second polarity/phase. Wire 896 connects electrodes 894 with connector element 897, and wire 898 connects electrodes 895 with connector element 897. Wires 896, 898 are appropriately insulated to avoid short circuit. Mated clip element 899 is connected to electrical connection 892 through a sealed hole through filter body 851. Connector elements 897, 899 are optional, and wires 896, 898 can be directly connected to electrical connection 892. Electrodes 894, 895 can be made from electrically conductive features placed along mesh 873, and mesh 873 can be formed from an insulating material, such as polymers (polyamines, polycarbonates, etc.) or ceramics (silicates, etc.) to avoid short circuits. An alternative view is shown in FIG. 39G. The numbers of electrodes and positioning can be altered as desired to achieve appropriate measurement values. With blood flowing through the filter, a certain current/impedance can be measured, which can alter if a clot is trapped by the mesh. Such a change of current or impedance measurement can indicate the presence of a clot, and the degree of change can provide information related to the amount or composition of the clot.

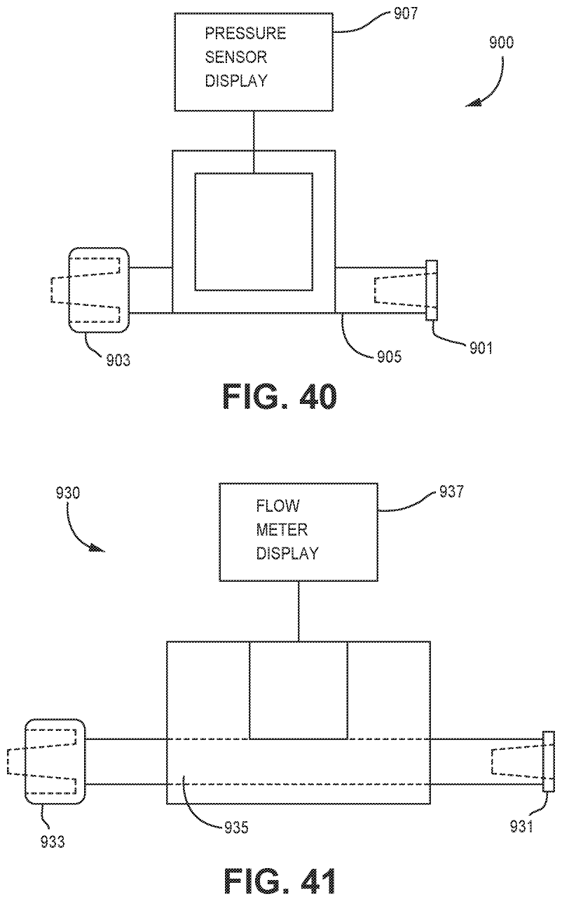

Referring to FIG. 40, pressure sensor 900 has a female luer fitting 901, a male luer fitting 903, and a channel 905 there between. Pressure sensor 900 may have a display 907 indicating the measured pressure of fluid passing through pressure sensor 900. In embodiments, display 907 may be integral with pressure sensor 900. In embodiments, display 907 may be a separate display unit, for example, connected via electrical connection or a wireless connection to pressure sensor 900. As described in more detail below, in embodiments, display 907 may be integrated into a multi-functional display that can contemporaneously display output from multiple sources during a procedure. Female luer fitting 901 and male luer fitting 903 are in fluid communication with fluid within channel 905.

Referring to FIG. 41, flow meter 930 has a female luer fitting 931, a male luer fitting 933, and a channel 935 there between. Flow meter 930 has a display 937 indicating the measured flow rate of fluid passing through flow meter 930. In embodiments, display 937 may be integral with flow meter 930. In embodiments, display 937 may be a separate display unit, for example, connected via electrical connection or wireless connection (such as blue tooth) to flow meter 930. As described in more detail below, in embodiments, display 935 may be integrated into a multi-functional display that can contemporaneously display output from multiple sources during a procedure. Readings from flow meter 930 can be simultaneously displayed on multiple display devices. Female luer fitting 931 and male luer fitting 933 are in fluid communication with fluid flowing through channel 935.

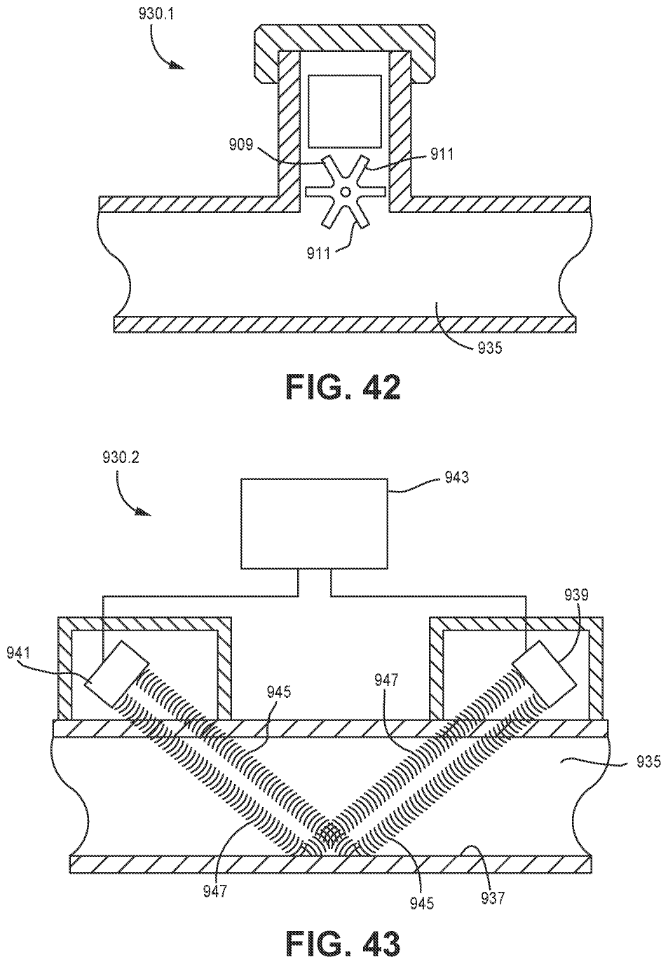

As shown in FIG. 42, in embodiments, flow meter 926 has a paddle wheel 909 positioned such that one or more paddles 911 extend partially into channel 905. Fluid flowing through channel 905 pushes the one or more paddles 911 causing paddle wheel 909 to rotate. A flow rate is associated with the rotational velocity of paddle wheel 909 as it turns.

In an alternative embodiment, as illustrated in FIG. 43, ultrasonic flow meter 928 has a first transceiver 939 and a second transceiver 941. First and second transceivers 939, 941 are in electrical communication with computational unit 943. First transceiver 939 emits a first ultrasonic signal 945 which reflects off an interior surface 937 of channel 935 with modulation from the fluid flow and is received by second transceiver 941. Second transceiver 941 emits a second ultrasonic signal 947 which reflects off an interior surface 937 of channel 935 and is received by first transceiver 939. In embodiments, first and second transceivers 939, 941 are ultrasonic transducers and/or ultrasonic sensors. Computational unit 943 receives output from first and second transceivers 939, 941. In embodiments, computational unit 943 can use output from first and second transceivers 939, 941 to calculate characteristics of fluid flowing through channel 935. For example, computational unit 943 can determine a flow rate of a fluid flowing through 935. Ultasonic flow meters are commercially available for adaptation to these purposes. For example, Dynasonics ultrasonic flow meters (such as Dynasonics DXN flow meter (Badger Meters, Inc., WI, USA) down to 0.5 inch diameter pipe) can be clipped onto a tube to measure flow rate based on Doppler ultrasound effect. Coriolis flow meter can connect directly to the tubing to allow flow through the device, and commercial coriolis flow meters are available, such as BFS Microfluidic Coriolis Flow Sensor (available from DARWIN Microfluidics) and controllable with ELVEFLOW Software.

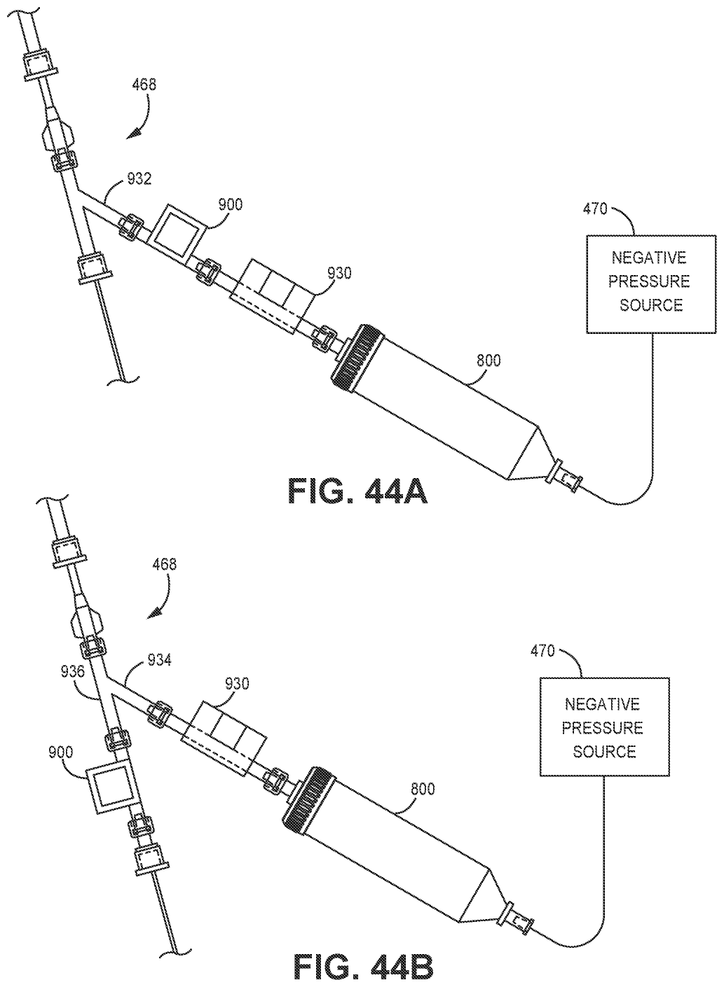

Examples of proximal fittings configurations comprising an filter 800, pressure sensor 900, flow meter 930, and negative pressure source 951 attached to the proximal fittings of an aspiration system as described herein are illustrated in FIGS. 44A and 44B. Referring to FIG. 44A, proximal fittings 468 are shown having a first branch 932 in fluid communication with pressure sensor 900, flow meter 930, filter 800, and negative pressure source 470. In such an arrangement, flow and pressure measurements are both taken on the first branch of the aspiration system, in line with negative pressure source 951. In an alternative arrangement, as shown in FIG. 44B, proximal fittings 468 are shown having a first branch 934 in fluid communication with flow meter 930, filter 800, and negative pressure source 951. A second branch 936 is shown in fluid communication with pressure sensor 900. Accordingly, a pressure of proximal fittings 468 may be determined independently of the branch attached to negative pressure source 951. Based on the teachings herein, various other configurations of the placement of the components can be achieved.

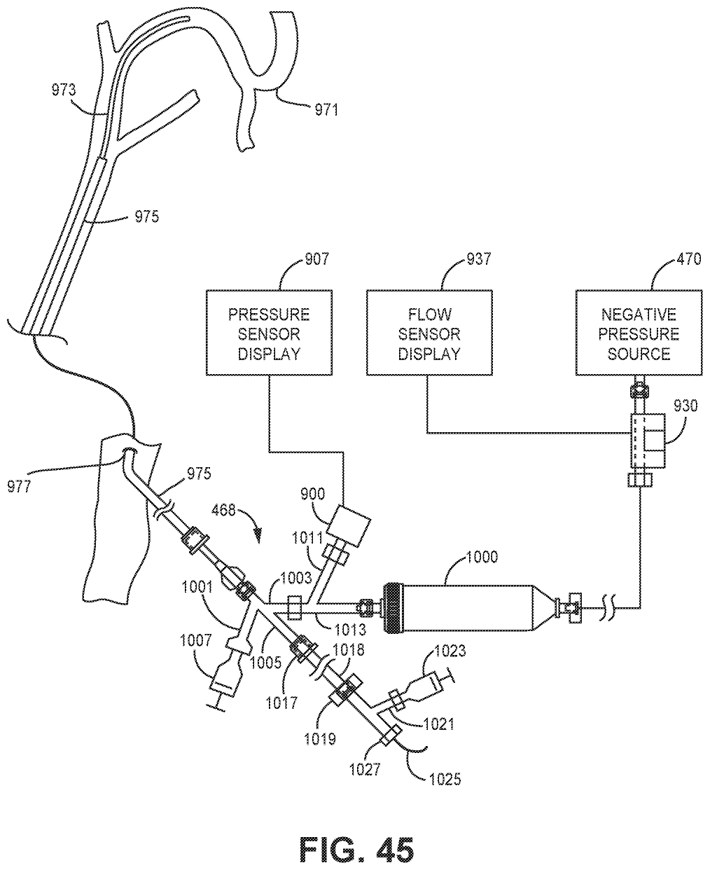

FIG. 45 depicts a fragmentary view of an embodiment of the aspiration system with elements of the catheters inserted into the patient. A distal portion of the aspiration system is shown in the neuro-vasculature 971 illustrating a tubular extension 973 extending from the guide catheter 975. A proximal portion of the aspiration system shows the guide catheter 975 extending proximally from the patient entry point 977 and the proximal fittings 468 of the aspiration system extending proximally from guide catheter 975. In embodiments, proximal fittings 468 have various branches providing desired functionalities as described in the several embodiments presented herein. In embodiments, branches 1001, 1003, 1005 may be multiple manifolds in various configurations such as a three branched manifold or two manifolds connected in series. In embodiments, first branch 1001 is distal to second branch 1003. In embodiments, second branch 1003 is distal to third branch 1005. In this particular embodiment, a first branch 1001 may be connected to fluid source 1007. Second branch 1005 may include pressure sensor 900, flow meter 930, filter 1000, and a negative pressure source 470. Pressure sensor 900 is connected to pressure sensor display 907 and flow meter 930 is connected to flow sensor display 937. In embodiments, second branch 1003 includes a Y-branch manifold having a first branch 1011 connected to pressure sensor and a second branch connected to filter 1000, flow meter 930, and negative pressure source 1009. In embodiments, first branch 1011 of Y-branch manifold is distal to second branch 1013 of Y-branch manifold. In embodiments, filter 1000 is distal to flow meter 930.

Extended hemostatic fitting 1018 is connected with third branch 1005 at connector 1017, and terminates with a hemostatic valve 1019. Extended hemostatic fitting 1018 can be combined with a docking branched manifold at hemostatic valve 1019, and suitable embodiments of a docking branched manifold are described above. In embodiments, extended hemostatic fitting 1019 may be combined with a docking branched manifold 1021. Docking branched manifold 1021 may have a first branch connected to a fluid source 1023. Control wire 1025 may extend from a second branch of branched manifold 1021 extending through hemostatic valve 1027.

The suction catheter system is generally appropriately sterilized, such as with e-beam or gas sterilization. The suction catheter system components can be packaged together or separately in a sealed package, such as plastic packages known in the art. The package will be appropriately labeled, generally according to FDA or other regulatory agency regulations. The suction catheter system can be packaged with other components, such as a guidewire, filter device, and/or other medical device(s). The packaged system generally is sold with detailed instructions for use according to regulatory requirements.

### Procedures Making Use of Treatment Systems

As indicated above, the medical systems comprising a suction catheter system described herein can be used with the suction catheter system as stand-alone treatment device, perhaps with a guidewire and/or other delivery support devices, or used with supplemental medical treatment devices for treatment of ischemic vessel blockage. In particular, in some embodiments, the suction system is used with an embolic protection device, and in additional embodiments, some form of clot engagement device, stent, balloon, atherectomy device or the like may also be used. In any case, a guidewire is generally used to provide access to the treatment site. The guide catheter portion of the suction catheter system may or may not be positioned prior to the introduction of the suction extension. The structures of the particular components are described in detail above, and are not repeated so that this section can focus on the use of the devices. The use of the alternative embodiments of the various fitting components can be adapted by a person of ordinary skill in the art based on the teachings herein.

In the procedures described below and generally herein, measurements from the instruments relating to pressure, flow and clot location can assist with the control of the procedure. In particular, the flow and pressure readings can provide significant information the catheter status, such as potential kinking, and clot interaction. The time dependence of the pressure and flow values can suggest differences between a kink and clot, as well as the size of a clot and movement of the clot. This information can assist with evaluating whether the catheter should be removed or if the procedure can continue with the current catheter status. If excessive flow is detected, a valve can be automatically closed to stop the flow. Thus, real time decision-making can be influenced by the measurements. The use of an automated valve, such as a solenoid valve, can provide another adjustment to facilitate the procedure and potentially improve efficiency.

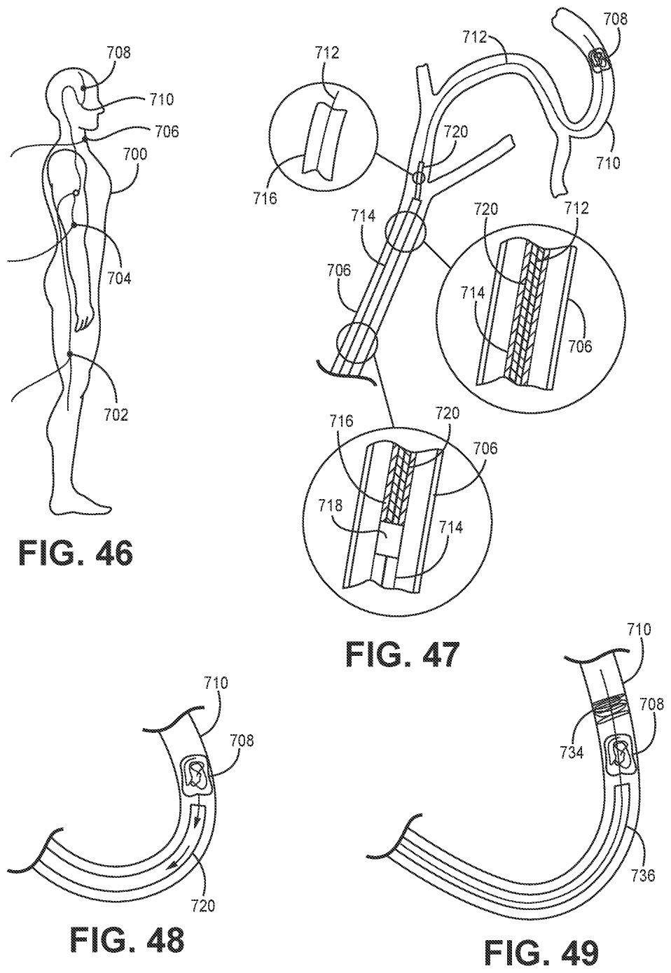

For the treatment of an acute ischemic stroke condition, referring to FIG. 46, a patient 700 is shown with three alternative access points into the vasculature, femoral artery 702, artery in the arm 704 or carotid artery in the neck 706. Regardless of the access point, the catheter and associated devices are guided to the left or right carotid artery to reach a clot 508 in a cerebral artery 710 of the brain. Referring to the schematic view in FIG. 47, clot 708 is shown in cerebral artery 710 with a guidewire 712 positioned with its distal tip past the clot. Guide catheter 714 is positioned over the guidewire within the carotid artery 706. Suction extension 716 with connecting section 718 within guide catheter 714 and tubular extension 720 extending from guide catheter 714 over guidewire 712. Referring to FIG. 48, tubular extension 720 can be advanced over the guidewire to a position near clot 708. Suction can be applied as shown with the flow arrows in the figure. Guidewire 712 may or may not be removed before suction is applied. Suction catheters have successfully removed clots responsible for ischemic stroke without further medical devices in the intervention. However, for more difficult clots, additional medical treatment devices can be used as described in detail below.

Using the embodiments of proximal fittings, such as shown above, adapted with pressure sensing capability, the initiation of suction as described in the context of FIG. 48 can be checked with respect to its efficacy. If appropriate flow is established since negative pressure is applied to the catheter system, the pressure in the proximal fittings can be in a suitable range. The precise ranges of expected pressures generally are dependent on the specific design of the suction extension, and the acceptable pressure range can be adjusted accordingly. In any case, the pressure can be confirmed in real time during the procedure for comparison with specifications adapted for the specific suction catheter components. If the pressure at the time immediately following the initiation of suction is closer to the negative pressure of the pump than expected based on the set acceptable range, the physician can withdraw the suction extension at least part way from the delivered configuration with or without stopping suction. A partial withdrawal can be used to try to unkink the suction extension without complete removal. As described further below, if proximal fittings are used that allow removal of the tubular extension for the patient without passing through a hemostatic valve, the tubular extension can be visually checked without exposing the tubular extension to the ambient atmosphere. After verifying that the tubular extension is ready for use or after replacing the suction extension, the suction extension can be redelivered.

When initiating the process, the system is generally primed with sterile saline to remove air from the aspiration system through to the pump. Pressure and flow measurements then relate to liquid parameters, such as the saline and/or blood as blood gets pulled into the system. When using the suction system to clear actual clots associated with acute ischemic stroke events, it is frequently found that the tubular extension becomes clogged itself prior to fully clearing the vessel. Therefore, it can be desirable to clear the clot form the tubular extension and reintroduce the suction extension back into the cerebral vessel to remove additional thrombus. The clearing and reintroduction can be repeated as necessary. The fittings described herein can facilitate this process, and the use of these fittings to effectuate this process are described further below. The desire to clear clots form the suction extension and reintroducing the suction extension may also be performed with the use of additional treatment structures as described in the following.

The use of a flow meter provide a significant additional parameter to guide the procedure. While pressure changes may provide some overlapping information, the additional flow measurements can provide additional guidance. If the flow drops, this can signal that the clot is lodged somewhere or that the suction extension is kinked. Depending on the stage of the procedure, the suction extension/aspiration catheter can be removed from the guide catheter and cleared of any clots. This then allows for the guide catheter to be checked if clear from any blockages. A sudden increase in flow can indicate that the clot has been removed. If the clot is in the filter, this can indicate advance of the procedure, but if the clot is not identified in the filter, the practitioner can carefully check likely alternative locations of the clot and proceed with caution in the procedure to avoid inadvertent redirecting the clot into the patient.

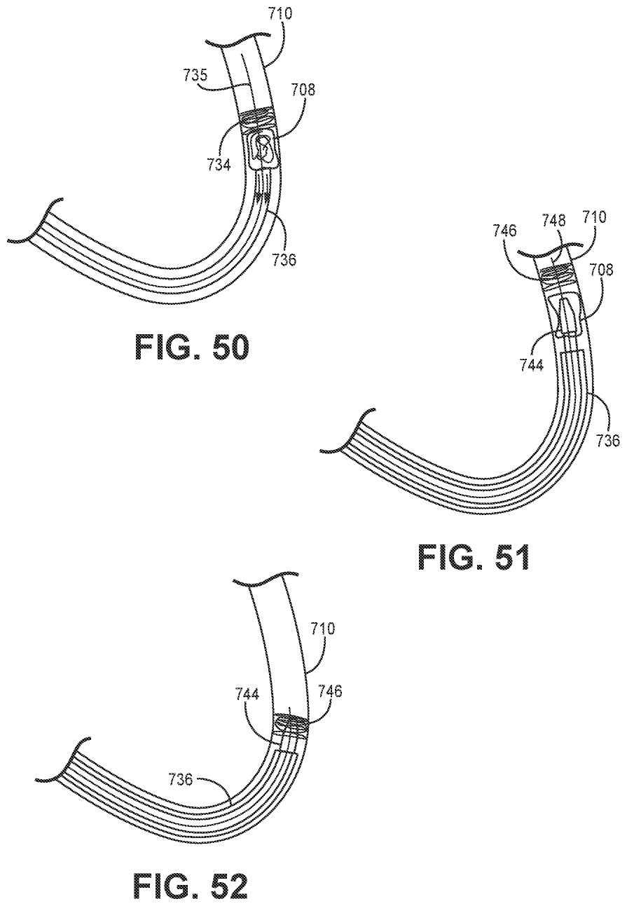

Referring to FIGS. 49 and 50, the use of a fiber-based filter device is shown in use along with the suction catheter system. As shown in FIG. 49, clot 708 is shown in cerebral artery 710 with a deployed fiber-based filter 734 supported on a guidewire 736 positioned with the filter deployed past the clot. Fiber-based filter 734 can have fiber elements extending essentially to the wall of the vessel, cerebral artery 710. Tubular extension 736 can be positioned with its distal tip just proximal to the clot, and the remaining portions of the suction catheter system are not shown in this view. Referring to FIG. 50, fiber-based filter 734 can be pulled toward tubular extension 736 with suction being applied to facilitate removal of clot 730. Clot 708 can be broken up and removed by suction, and/or all or a portion of clot 708 can be pulled into tubular extension 736 optionally along with all or part of the fiber-based filter, and/or all or a portion of clot 708 can be held to the opening of tubular extension 736 with the fiber-based filter holding the clot. In any case, once the clot is appropriately stabilized, the devices and any clot still within the vessel or catheter can be removed from the patient. The removal of the devices is described further below.

The further use of an additional medical device to facilitate clot removal is shown in FIGS. 51 and 52. As shown in FIG. 51, clot 708 is shown in cerebral artery 710 with a medical treatment device 754 positioned at the clot and deployed fiber-based filter 756 supported on a guidewire 758 positioned with the filter deployed past the clot. Suitable medical treatment devices for clot engagement are described above. The selected medical treatment device is deployed generally with protection from the deployed fiber-based filter and optionally with suction. Once the clot is engaged with the medical treatment device, the recovery of the remaining portions of the clot and the medical treatment devices can be removed as shown in FIG. 52, similarly to the process shown in FIG. 51. In particular, the medical treatment device can be removed, although portions such as a stent may be left behind, and the removal can precede or can be done in conjunction with removal of a filter in the blood vessel and/or remaining fragments of clot. All or a portion of clot 708, if not already broken up and removed with suction can be pulled into tubular extension 736 optionally along with all or part of the fiber-based filter, and/or all or a portion of clot 708 can be held to the distal opening of tubular extension 736 with the fiber-based filter holding the clot. Again, once the clot is appropriately stabilized, the devices and any clot still within the vessel or catheter can be removed from the patient. The use of a plurality of additional medical treatment devices can be performed through extension of the procedure outlined above to repeat steps involving the additional medical device.

Also, for the embodiments in FIGS. 47-52, a pressure sensor connected to the proximal fittings can be used to guide the procedures. If the pressure in the proximal fittings increases to a pressure outside of a target range when negative pressure is initiated, appropriate remedial attention can be applied to remove a kink, or replace/clear the suction extension, or other appropriate attention. Furthermore, after suction is applied and the clot seems to have been addressed, the pressure in the proximal fittings can be checked to evaluate the status of the clot and the catheter, such as whether or not the clot is trapped at the distal end of the suction extension. Appropriate care can be taken based on the pressure in the proximal fittings.

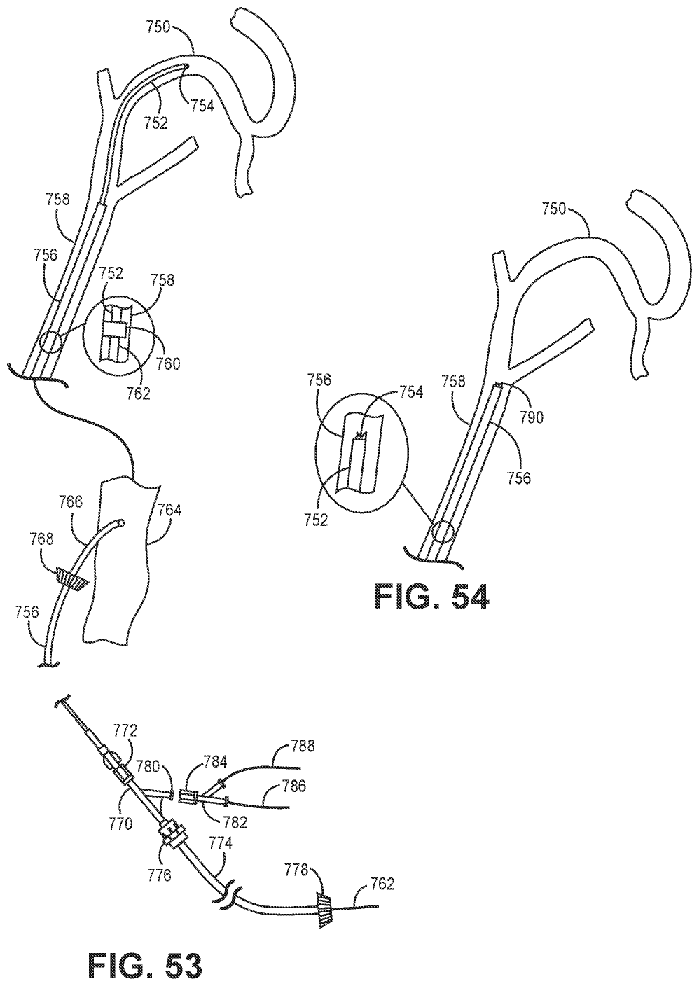

FIG. 53 depicts the suction treatment system following treatment of a clot in cerebral artery 750. Tubular extension 752 is positioned with its distal tip in cerebral artery 750 and thrombus 754 may or may not be present at the opening. Guide catheter 756 is located with its distal end in carotid artery 758. A section of the interior of guide catheter 756 is shown in a balloon insert of FIG. 53. Connecting section 760 of suction extension 752 is within guide catheter 756 with control wire 762 extending in a proximal direction. The patient's leg 764 is shown with an introducer sheath 766 extending from the leg with a hemostatic valve 768. Guide catheter 756 extends out from hemostatic valve 768. Y-branch manifold 770 is connected to the distal end of guide catheter 756 at connector 772. Extended hemostatic fitting 774 is connected with Y-branch manifold 770 at connector 776, and terminates with a hemostatic valve 778. Control wire 762 extends from hemostatic valve 778. Y-branch manifold 770 has a connector 780 that can be connected to a further Y-branch manifold 782 with connector 784 for connection to connector 780. Y-branch manifold can be connected to a negative pressure line 786 that can be connected to a pump or other negative pressure device, and to a pressure sensor line 788 that can be connected to an appropriate pressure sensor such as those of FIGS. 27-29. The fittings of FIG. 53 can be combined with a docking branched manifold at hemostatic valve 778, and suitable embodiments of a docking branched manifold are described above. The combination of Y-branched manifold 770 and extended hemostatic fitting 774 can be considered components of the first fitting elements.

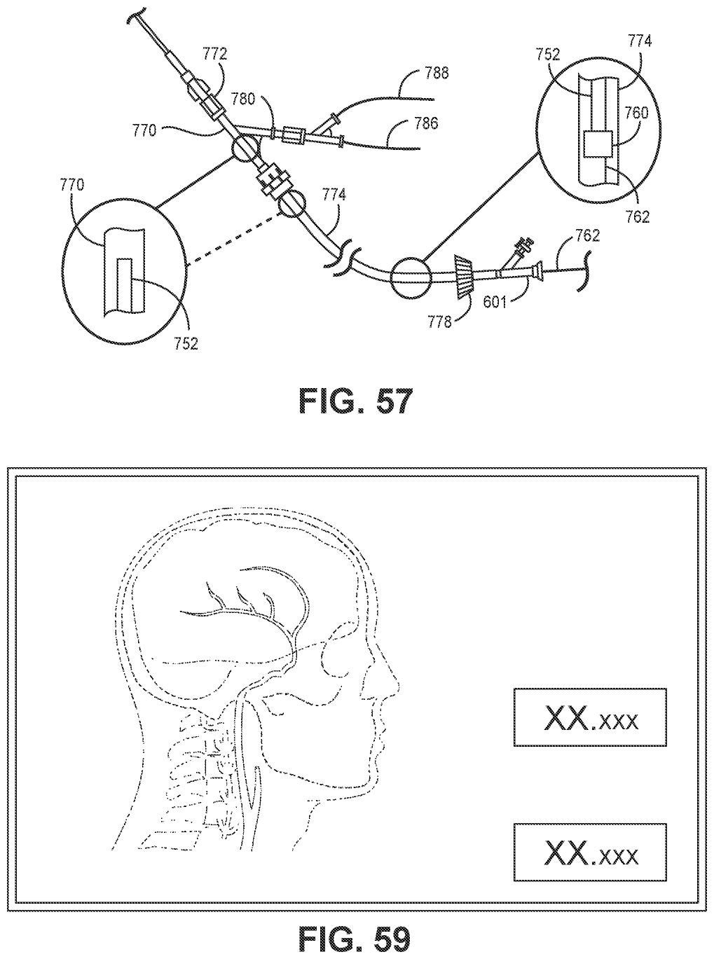

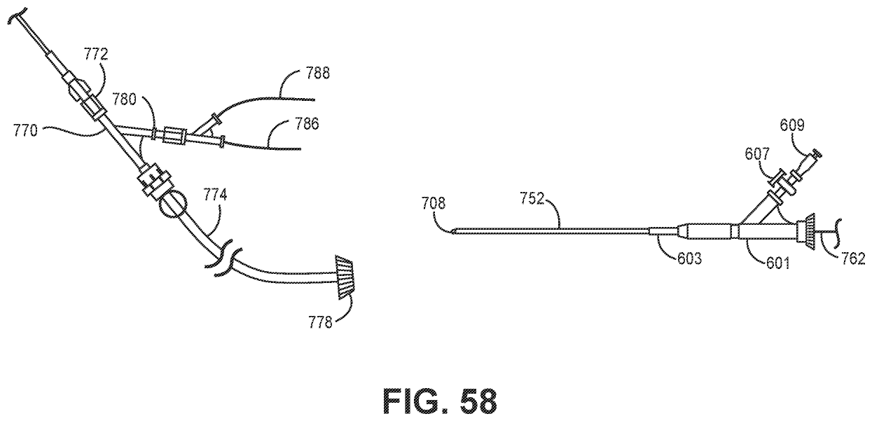

At the stage of the procedure shown in FIG. 48 (assuming thrombus is removed to the extent desired) and 53, procedural steps can be initiated for gradual removal of the devices from the patient. FIGS. 54-56 show the removal process using extended fittings that provide for the removal of the suction extension completely form the guide catheter behind a hemostatic valve. FIGS. 57 and 58 depict the use of the docking Y-fitting providing for the efficient clearing and reintroduction of the suction extension. It can be advantageous to maintain the guide catheter in position while removing the other components and verifying the success of the procedure. Generally, it is desired to keep the guide catheter in place until the procedure is to be completely ended since the guide catheter placement involves significant effort. As noted above, the suction extension may be removed, cleared of clots, and reintroduced for additional thrombus removal prior to termination of the overall procedure. This removal and reintroduction of the suction extension can be performed with the guide catheter fixed in place. Pressure readings at the proximal fittings can provide useful information regarding the status of potential blockages of flow into suction extension 752, although other more qualitative evaluations can be performed such as the termination of fluid flow into the pump.

Referring to FIG. 54, guide catheter 756 is still in place in carotid artery 758 and cerebral artery 750 is clear of devices and clot. Referring to the balloon figure insert associated with FIG. 54, a further enlarged sectional view shows the distal end of suction extension 752 within the interior of guide catheter 756. Thrombus may or may not be associated with the distal end of guide catheter 756 (thrombus 790), which can be deposited there when suction extension 752 is withdrawn into guide catheter 756, and/or at the distal end of suction extension 752 (thrombus 754). Again, a pressure reading in the proximal fittings can provide useful information on potential thrombus blocking flow through the catheter system to the negative pressure device, such as a pump.

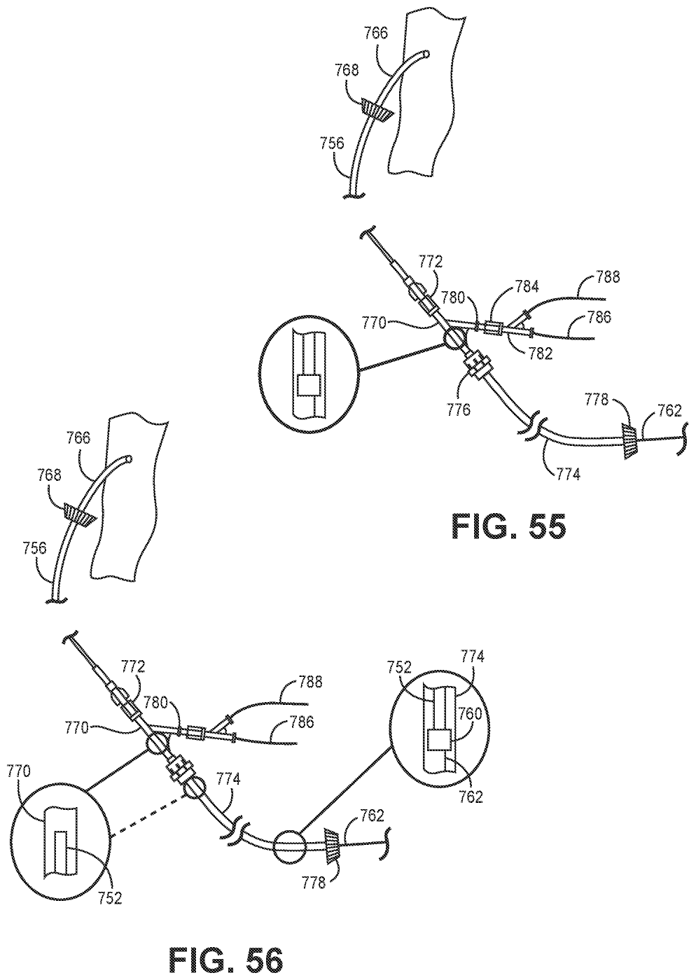

Referring to FIG. 55, upon further withdrawal of suction extension 752 from the patient, a balloon figure insert shows a further enlarged section view with connecting section 760 of suction extension 752 within T-branch manifold 770. With this configuration, a continuation of application of negative pressure would draw fluid from guide catheter 756 rather than through suction extension 752. Whether or not suction extension 752 is plugged, this configuration can provide addition possibility of removal of thrombus 790 at the end of guide catheter 756, and the suction can further stabilize thrombus 790, if any, for further portions of the procedure. At this stage of the procedure, the pressure in the proximal fittings can provide information on the flow of liquid into guide catheter 756.

The complete removal of suction extension 752 from guide catheter 756 is shown in FIG. 56. A distal balloon figure insert in FIG. 56 shows a further expanded section view with the distal end of suction extension 752 within T-branch manifold 770, although the distal end of suction extension 752 can be withdrawn fully into extended hemostatic fitting 774 as noted by the dashed line connected to the balloon figure insert. A proximal balloon figure insert in FIG. 56 shown a further expanded sectional view with connection section 760 within extended hemostatic fitting 774 in a position distal to hemostatic valve 778. Again, pressure within proximal fittings can be useful to provide information during this part of the procedure.

While guide catheter 756 can be removed from the patient following treatment of the clot, it can be desirable to at least partially remove suction extension 752 relative to its deployed location with the guide catheter in position to reduce the risk of embolization of thrombus that may be trapped in association with the aspiration system components but not yet fully removed from the patient. FIGS. 44-46 depict three stages of suction extension removal at which time it can be selected to remove guide catheter 756 from the patient, generally through hemostatic valve 768 of introducer 766. With the distal end of suction extension 752 within guide catheter 756, as shown in FIG. 54, any thrombus associated with suction extension 752 is within guide catheter 756 so that it is less likely to involve embolization. Referring to FIG. 55, as noted above, connection section 760 within Y-branch manifold 770, suction is applied directly to guide catheter 756 lumen regardless of whether or not suction extension 752 is clogged, and this direct application of suction to guide catheter 756 provides an added degree of safety with respect to reducing chances of embolization. Furthermore, complete removal of suction extension 752 from guide catheter 756, as shown in FIG. 56, provides additional safety against embolization of thrombus associated with suction extension 752. As shown in FIG. 56, suction extension 752 remains in isolation behind a hemostatic valve 778, and this configuration provides for desirable control of pressures within guide catheter 756 that further reduces risk of embolization as well as contamination.

FIG. 57 shows a docking branched manifold 601 with a distal end inserted through hemostatic valve 778 and control wire 762 extending proximally. As discussed above, the use of docking branch manifold 601 permits efficient clearing and reintroduction of the suction extension. As shown in FIG. 57, suction extension 752 is removed from guide catheter 756 but still remains in isolation behind hemostatic valve 778. When suction extension 752 is clogged, the configuration shown provides added safety against embolization of thrombus. However, to safely reintroduce suction extension 752 into a patient, clogs should first be cleared from suction extension 752. As discussed above, control wire 762 may be used to dock suction extension 752 in docking branch manifold 601.

FIG. 58 shows both docking branch manifold 601 and suction extension 752 fully withdrawn from isolation behind hemostatic valve 778. In this configuration, a proximal end of suction extension 752 is docked within input tubular segment 603. Further, at least a portion of clot 708 is shown clogging a distal end of suction extension 752. Reintroduction of suction extension 752 into a patient while clot 708 is clogging a portion of suction extension 752 could be unsafe. Accordingly, with suction extension 752 fully removed from hemostatic valve 778, valve 607 may be opened so that a positive pressure device such as syringe 609 may inject fluid to flush clot 708 from suction extension 752. Once suction extension 752 is cleared, suction extension 752 may be reinserted through hemostatic valve 778. Sterile procedures can be used to maintain suction extension 752 in a sterile condition for reintroduction into the patient. In some procedures, cleared suction extension 752 may be fully reintroduced into a patient for retrieval of additional emboli. As noted above, the docking manifold can be configured to deliver aspiration, contrast fluid or other fluids to facilitate performance of the procedure. For these additional or alternative embodiments, the procedures can be straightforwardly revised.

FIG. 59 depicts a video monitor 680 displaying a real time x-ray image 682 of the patient at the site of the thrombus removal along with the pressure value 684 and the flow rate 686. Through having all of these images simultaneously visible, the health care provider can assess all of the information to make a decision regarding next steps of the procedure.

Bench testing and calculations were performed to evaluate the general suction performance of the use of a suction extension interfaced with a guide catheter and for other commercial suction catheters. These results are described in the '938 application, and are incorporated herein by reference.

### Additional Inventive Comments

A. An aspiration catheter system comprising:

a guide catheter comprising a proximal hub and a tubular section connected to the proximal hub with a lumen extending from the proximal hub to a distal opening;

an aspiration catheter comprising a connecting section, a distal aspiration section with an aspiration lumen and a distal aspiration opening, and a proximal control structure, wherein when positioned in the guide catheter as a suction extension, the distal aspiration section can extend from the distal opening of the guide catheter with the connecting section engaging with the inner wall of the guide catheter within the lumen and with the proximal control structure extending through the hub of the guide catheter, and wherein the connecting section comprises a tubular element with a lumen open at a proximal end and fluidly connecting with the aspiration lumen of the distal aspiration section, a framework around the tubular element having a low profile configuration and a higher profile engagement configuration and wherein a transition from the low profile configuration to the higher profile engagement configuration can be actuated through engagement with the framework using the proximal control structure having a translatable element between a proximal end and the framework, or through release of constraints on the framework, the framework being self-actuating; and

proximal fittings connected to the proximal hub of the guide catheter and comprising a manifold with a hemostatic valve configured to provide for passage of the proximal control structure to pass through the hemostatic valve when the distal aspiration section is extending in a distal orientation from the distal opening of the guide catheter, and with a branch connecting to an aspiration source with a configuration to apply negative pressure along an aspiration lumen extending from the negative pressure source through the manifold, a portion of the guide catheter to the lumen of the aspiration catheter to apply suction at the distal aspiration opening.

A1. The aspiration catheter system of claim A wherein the framework is covered with a membrane.
A2. The aspiration catheter system of claim A1 wherein the membrane comprises an elastomer.
A3. The aspiration catheter system of claim A wherein the outer diameter of the framework relative to the catheter axis has a maximum difference between the engagement configuration and the low profile configuration of about 3Fr.
A4. The aspiration catheter system of claim A wherein delivery of electrical current to the framework results in thermal actuation causing the framework to transition between the low profile configuration and the engagement configuration.
A5. The aspiration catheter system of claim A wherein the proximal control structure comprises a corewire within an overtube and movement of the corewire relative to the overtube actuates the framework between the low profile configuration the engagement configuration.
A6. The aspiration catheter system of claim A5 wherein pushing the corewire causes the framework to transition from the low profile configuration to the engagement configuration.
A7. The aspiration catheter system of claim A5 wherein pulling the corewire causes the framework to transition from the low profile configuration to the engagement configuration.
A8. The aspiration catheter system of claim A5 wherein the corewire is configured to transition the framework from the engagement configuration to the low profile configuration through reversal of the relative movement of the corewire.
A9. The aspiration catheter system of claim A wherein the framework self-actuates to transition from the low profile configuration to the engagement configuration upon release of constraints on the self-actuation.
A10. The aspiration catheter system of claim A9 wherein a sheath surrounding the framework maintains the framework in the low profile configuration; and wherein the framework self-actuates when the sheath is transitioned to release constraints on the framework.
A11. The aspiration catheter system of claim A9 wherein the self-actuating framework comprises thermally activated shape memory metal that responds to body temperature.
A12. The aspiration catheter system of claim A wherein the extension in the engagement configuration is adjustable.
B. A method for applying aspiration within a bodily vessel with an aspiration catheter system comprising a guide catheter, an aspiration catheter comprising an engagement section, a proximal control structure, and a distal aspiration tube, and proximal fittings comprising a hemostatic valve and a connection to an aspiration source, wherein the engagement section has a low profile configuration and an extended configuration, the method comprising:

positioning a distal aspiration opening of the aspiration catheter at a desired position within a vessel within a patient;

after positioning the aspiration catheter, transitioning the engagement section to an extended configuration forming a seal with the wall of the guide catheter, wherein the transitioning comprises delivering electrical current through the proximal control structure or moving a corewire within the proximal control structure; and

applying suction at the distal aspiration opening to perform a desired procedure in the vessel.

B1. The method of claim B wherein the desired position places the distal aspiration opening in a cerebral artery to provide effective aspiration to effectuate removal of a clot.
B2. The method of claim B wherein x-ray imaging is used to position the distal aspiration opening at the desired position.
B3. The method of claim B wherein delivery of a current to the engagement section causes the engagement section to transition from the low profile configuration to the extended configuration.
B4. The method of claim B wherein manipulating a corewire connected to the engagement section causes the engagement section to transition from the low profile configuration to the extended configuration.
B5. The method of claim B wherein drawing back a sheath surrounding the engagement section allows the engagement section to transition from the low profile configuration to the extended configuration
B6. The method of claim B further comprising repositioning the engagement section after transitioning the engagement section to an engagement configuration.
B7. The method of claim B further comprising delivering a device through the aspiration catheter.
B8. The method of claim B7 wherein the delivering of the device is performed with the engagement section in an extended configuration and prior to applying suction.
B9. The method of claim B7 wherein the device comprises a filter.
B10. The method of claim B9 wherein the filter comprises a fiber based filtration matrix.
B11. The method of claim B7 wherein the device is a balloon infusion catheter.
B12. The method of claim B7 wherein the device is a stent retriever.
B13. The method of claim B7 further comprising delivering a second device through the aspiration catheter.
B14. The method of claim B13 wherein the first device is not removed prior to delivering second device.
B15. The method of claim B further comprising:

withdrawing the aspiration catheter into the guide catheter such that the distal aspiration is within the lumen of the guide catheter, while aspiration continues.

B16. The method of claim B15 wherein the framework is extended while withdrawing the aspiration catheter.
B17. The method of claim B further comprising monitoring the pressure to evaluate the status of the aspiration.
B18. The method of claim B further comprising monitoring the flow with a flow meter to evaluate the status of the aspiration.
C. An aspiration thrombectomy system comprising:

an aspiration catheter assembly comprising a suction lumen extending from a proximal end to a distal opening, wherein the proximal end comprises a connector;

fittings comprising a branched manifold with a first branch comprising a hemostatic valve and a second branch comprising a connector, wherein the branched manifold is attached to the connector of the aspiration catheter assembly;

a pump; and

a conduit connected to the pump and to the connector of the second branch, the conduit comprising tubing and a filter having an inlet and an outlet connected to the tubing, wherein the filter comprises a filtration structure comprising electrodes for impedance/conductivity measurements.

C1. The aspiration thrombectomy system of claim C wherein the electrodes of the filtration structure are connected to a controller that provides current to the electrodes and measures fluctuations to the current to evaluate the electrical properties of material contacting the filter.
C2. The aspiration thrombectomy system of claim C wherein the filter comprises a filter body defining an interior chamber along a flow path between the inlet and the outlet; and a filter element configured to fit within the interior chamber such that flow between the inlet and the outlet flows through the filter element, wherein the inlet is associated with a first connector and the outlet is associated with a second connector.
C3. The aspiration thrombectomy system of claim C2 wherein the filter element comprises a mesh screen configured in the interior chamber to separate flows between the inlet and the outlet such that flow from the inlet to the outlet passes through the mesh screen.
C4. The aspiration thrombectomy system of claim C3 wherein the inlet and outlet are positioned at a selected position along the cap circumference, the cap comprising a first channel extending from the inlet to a central portion of the cap and a second channel extending from the outlet to interior of the filter body such that fluid flowing through the inlet passes through filter element before exiting through the second channel.
C5. The aspiration thrombectomy system of claim C wherein the filter is configured to retain clots flowing through the conduit.
C6. The aspiration thrombectomy system of claim C wherein the filter is adjacent the fittings.
C7. The aspiration thrombectomy system of claim C wherein the filter is configured to be manually inspected for debris.
C8. The aspiration thrombectomy system of claim C wherein in the filter comprises a filter element secured in a chamber below a cap.
C9. The aspiration thrombectomy system of claim C8 wherein the cap is separable from the chamber such that a filter element clogged with debris is replaceable with a clean filter element.
C10. The aspiration thrombectomy system of claim C8 wherein the filter element is cylindrical such that a diameter of the filter element is greater than a height of the filter element.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understood that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated.

What is claimed is:

1. An aspiration catheter system comprising:
an aspiration catheter with a distal aspiration opening and lumen ending at the distal aspiration opening, wherein the wall of the aspiration catheter comprises a pressure sensor with an output value related to pressure at the pressure sensor; and
a proximal fitting connected with an aspiration source to provide negative pressure at the distal aspiration opening.

2. The aspiration catheter system of claim 1 wherein the pressure sensor is configured to measure pressure within the lumen.

3. The aspiration catheter system of claim 1 wherein the pressure sensor is configured to measure pressure exterior to the aspiration catheter.

4. The aspiration catheter system of claim 1 wherein output of pressure related value is transmitted by a wire extending in a proximal direction.

5. The aspiration catheter system of claim 1 wherein output of pressure related value is transmitted wirelessly.

6. The aspiration catheter of claim 1 wherein the aspiration catheter comprises a tubular element and a hub that connects with the proximal fitting to form an aspiration lumen from the proximal fittings to the distal aspiration opening.

7. The aspiration catheter system of claim 1 further comprising a guide catheter and wherein the aspiration catheter comprises a connecting section, a distal aspiration section, and a proximal control structure with the lumen extending through the connecting section and the distal aspiration section,
wherein when the aspiration catheter is positioned in a lumen of the guide catheter as a suction extension, the distal aspiration section can extend from a distal opening of the guide catheter with the connecting section engaging with an inner wall of the guide catheter and with the proximal control structure extending through the proximal fittings.

8. The aspiration catheter system of claim 7 wherein at least a portion of the connecting section has a non-cylindrical cross section with a major outer diameter and a minor outer diameter smaller than the major outer diameter.

9. The aspiration catheter system of claim 7 wherein the connecting section comprises a tubular element with a lumen open at a proximal end and fluidly connecting with the aspiration lumen of the distal aspiration section, a framework around the tubular element having a low profile configuration and a higher profile engagement configuration and wherein a transition from the low profile configuration to the higher profile engagement configuration can be actuated through engagement with the framework using the proximal control structure having a translatable element between a proximal end and the framework, or through release of framework using a tether to transition a constraint, the framework being self-actuating.

10. The aspiration catheter system of claim 1 wherein a flow meter connected to the proximal fittings is configured to measure flow in the aspiration lumen.

11. The aspiration catheter system of claim 1 further comprising a valve and an electronic controller, wherein the valve is configured to control flow through the tubing and wherein the electronic controller controls operation of the valve.

12. The aspiration catheter system of claim 11 wherein the controller configured to receive input of a value related to the pressure sensor reading to factor into valve control.

13. The aspiration catheter system of claim 11 wherein the valve can adjust to intermediate positioned between fully open and fully closed.

14. A method for performing aspiration from a bodily vessel, the method comprising:
apply aspiration with the aspiration catheter of claim 1 within a vessel;
monitoring pressure to obtain a pressure value exterior to the aspiration catheter within the vessel and/or within the lumen; and
adjusting aspiration negative pressures or stopping aspiration based on the measured pressure.

15. An aspiration catheter system comprising:
an aspiration catheter assembly comprising a lumen extending from a proximal end with a connector, to a distal opening;
fittings comprising a branched manifold with a first branch comprising a hemostatic valve and a second branch comprising a connector;
a pump;
a conduit connected to the pump and to the connector of the second branch;
a flow meter connected the connector or to the fittings configured to measure flow from or to the aspiration catheter; and
a controller comprising one or more displays configured to display values related to the flow.

16. The aspiration catheter system of claim 15 wherein the flow meter is configured interfacing with the connector of the aspiration catheter assembly.

17. The aspiration catheter system of claim 15 wherein the flow meter is configured within the fittings.

18. The aspiration catheter system of claim 15 wherein output of flow related value is transmitted by a wire connected to the controller.

19. The aspiration catheter system of claim 15 wherein output of flow related value is transmitted wirelessly to the controller.

20. The aspiration catheter system of claim 15 wherein the aspiration catheter assembly comprises a catheter comprising a tubular element and a hub that connects with the proximal fitting to form an aspiration lumen from the proximal fittings to the distal aspiration opening.

21. The aspiration catheter system of claim 15 wherein the aspiration catheter assembly comprising a guide catheter and an aspiration catheter and wherein the aspiration catheter comprises a connecting section, a distal aspiration section and a distal aspiration opening, and a proximal control structure with an aspiration lumen extending through the connecting section and the distal aspiration section,
wherein when the aspiration catheter assembly is positioned in the lumen of the guide catheter as a suction extension, the distal aspiration section can extend from the distal opening of the guide catheter with the connecting section engaging with the inner wall of the guide catheter and with the proximal control structure extending through the proximal fittings.

22. The aspiration catheter system of claim 21 wherein at least a portion of the connecting section has a non-cylindrical cross section with a major outer diameter and a minor outer diameter smaller than the major outer diameter.

23. The aspiration catheter system of claim 21 wherein the connecting section comprises a tubular element with a lumen open at a proximal end and fluidly connecting with a lumen of the distal aspiration section, a framework around the tubular element having a low profile configuration and a higher profile engagement configuration and wherein a transition from the low profile configuration to the higher profile engagement configuration can be actuated through engagement with the framework using the proximal control structure having a translatable element between a proximal end and the framework, or through release of framework using a tether to transition a constraint, the framework being self-actuating.

24. The aspiration catheter of claim 15 wherein a pressure sensor connected to the aspiration catheter assembly is configured to measure pressure in the suction lumen.

25. The aspiration catheter system of claim 14 further comprising a valve and an electronic controller, wherein the valve is configured to control flow through the tubing and wherein the electronic controller controls operation of the valve.

26. The aspiration catheter system of claim 25 wherein the controller configured to receive input of a value related to the flow meter reading to factor into determination of valve control.

27. The aspiration catheter system of claim 25 wherein the valve can adjust to intermediate positioned between fully open and fully closed.
